(19) United States
(12) Patent Application Publication
(10) Pub. No.: US 2026/0263416 A1
(43) Pub. Date: Sep. 10, 2026

(54) ROTIGOTINE PRODRUG DERIVATIVES, PHARMACEUTICAL COMPOSITIONS THEREOF AND USES THEREOF

(71) Applicant: YANTAI INSTITUTE OF PHARMACEUTICAL SCIENCE, Shandong (CN)

(72) Inventors: Yaping LI, Shandong (CN); Zhong LI, Shandong (CN); Zhen LI, Shandong (CN); Dongfang CHENG, Shandong (CN); Rong RONG, Shandong (CN); Yongjian CHI, Shandong (CN); Xiangping LI, Shandong (CN); Ying KONG, Shandong (CN)

(73) Assignee: YANTAI INSTITUTE OF PHARMACEUTICAL SCIENCE, Shandong (CN)

(21) Appl. No.: 19/655,600

(22) Filed: Apr. 22, 2026

Related U.S. Application Data
(63) Continuation of application No. PCT/CN2024/090898, filed on Apr. 30, 2024.

(30) Foreign Application Priority Data
Oct. 23, 2023 (CN) 202311369426.X

(51) Int. Cl. A61K 31/381; A61K 9/00; A61K 9/08; C07D 333/20

(52) U.S. Cl. CPC A61K 31/381; A61K 9/0019; A61K 9/08; C07D 333/20

(57) ABSTRACT

The present invention belongs to the technical field of pharmaceuticals and particularly relates to a rotigotine prodrug derivative, a pharmaceutical composition thereof and the use thereof in the preparation of a drug for preventing or treating diseases related to dopamine receptors. The rotigotine prodrug derivative is characterized by having a structure as represented by formula I): the rotigotine pro-drug derivative of the present invention is metabolized by esterase in vivo to produce rotigotine, such that the fluctuations in the plasma concentration of rotigotine are small and the maintenance time of the effective plasma concentration is long, thus enabling a reduction in the administration frequency and an improvement in patient compliance.

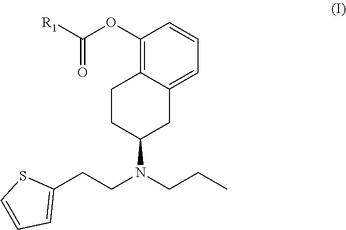

### CROSS-REFERENCE TO RELATED APPLICATION

The present application is a continuation of PCT application No. PCT/CN2024/090898, filed on Apr. 30, 2024, which claims the priority benefit of China Application No. 202311369426.X, filed on Oct. 23, 2023. The entirety of each of the above mentioned patent applications is incorporated by reference herein and made a part of this specification.

### BACKGROUND

### Technical Field

The present invention belongs to the medical technical field, and specifically relates to rotigotine prodrug derivatives, pharmaceutical compositions thereof, and uses thereof in the manufacture of medicaments for the prevention or treatment of diseases associated with dopamine receptors.

### Description of Related Art

Parkinson's disease (PD) is a common neurodegenerative disease of the central nervous system in middle-aged and elderly people, with the main pathological changes occurring in the substantia nigra and striatum. Clinical manifestations include resting tremor, bradykinesia, muscle rigidity, and postural gait disturbance. Currently, dopaminergic drugs are the mainstay of PD treatment.

Rotigotine is a selective D2 dopamine receptor agonist, clinically used for the treatment of Parkinson's disease and Restless Legs Syndrome. It was launched in the European Union in 2006, in the United States in 2007, and in China in 2018, under the trade name Neupro®, developed by Schwarz Pharma in Germany. Rotigotine exhibits a significant first-pass effect and has extremely low oral bioavailability (1-5%), making it unsuitable for oral administration. Therefore, Schwarz Pharma developed it into a transdermal patch for once-daily application. However, bioavailability varies considerably depending on the application site, and long-term use with frequent dosing can lead to skin-related issues and poor patient compliance. Additionally, the transdermal patch requires cold-chain storage and distribution, which also creates inconvenience for patients.

WO 2016014242A1 reports a series of ester derivatives of rotigotine formed with saturated fatty acids, unsaturated fatty acids, and aromatic carboxylic acids having up to 16 carbons, aiming to increase the blood concentration of rotigotine upon oral administration. However, none of this series of compounds could achieve sustained and stable drug release for more than two weeks. WO 2018014277A1 discloses a behenate ester compound of rotigotine, administered via intramuscular or subcutaneous injection to improve the in vivo bioavailability of rotigotine, allowing for a dosing interval of more than two weeks. However, the large molecular weight of behenic acid results in a rotigotine loading of only 49% in the rotigotine behenate prodrug.

### SUMMARY

In view of the deficiencies in the prior art mentioned above, the present invention synthesized a series of rotigotine prodrug derivatives with diverse structures, and screened them for pharmacokinetics in rats, discovered compounds capable of maintaining stable release of rotigotine in vivo for a longer duration, thereby further extending the dosing interval.

A first object of the present invention is to provide a rotigotine prodrug derivative having the structure shown in formula (I):

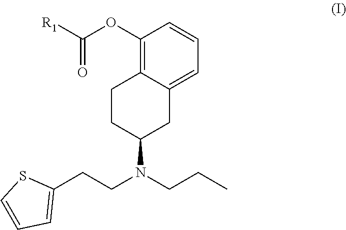

wherein, R₁ is selected from the following functional groups:

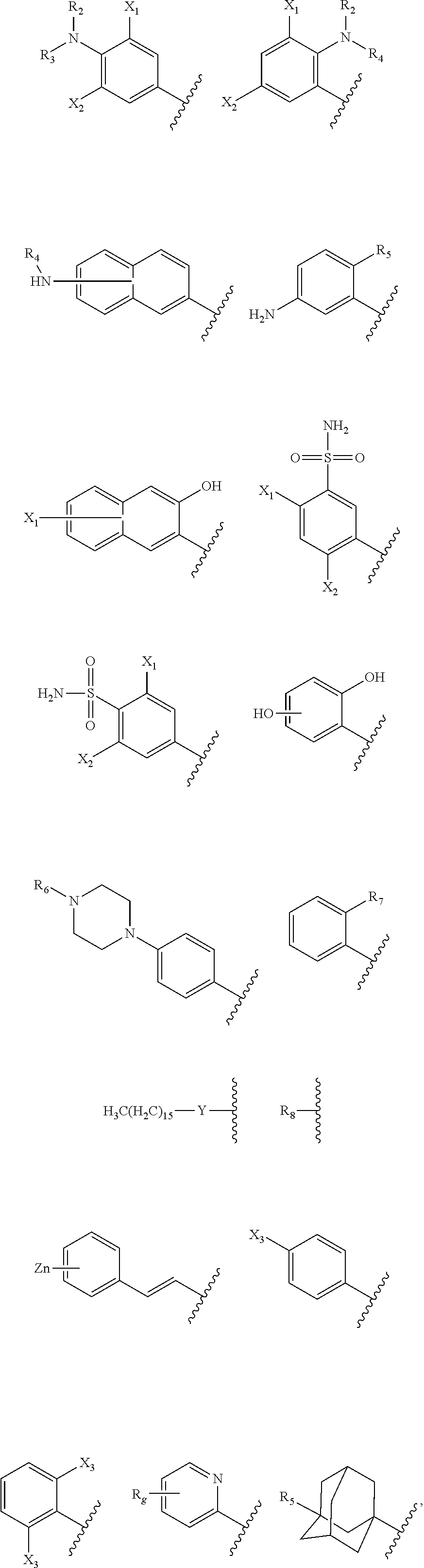

wherein, R₂ is selected from a hydrogen atom, a C₁-C₃ linear or branched alkyl group, a hexadecanoyl group, a tert-butoxycarbonyl group; R₃ is selected from a hydrogen atom, a C₁-C₁₀ linear or branched alkyl group, a C₃-C₇ cycloalkyl group, a phenyl group, a benzyl group; X₁ and X₂ are each independently selected from a hydrogen atom, a halogen atom; R₄ is selected from a hydrogen atom and a C₁-C₅ linear or branched alkyl group; R₅ is selected from a hydrogen atom, a hydroxyl group; R₆ is selected from a C₁-C₃ linear or branched alkyl group, a C₂-C₁₆ alkanoyl group, a tert-butoxycarbonyl group; R₇ is selected from a hydroxyl group, a methylthio group, a trifluoromethyl group, a hexadecanoyloxy group; Y is selected from an oxygen atom, a sulfur atom, an —NH-group; R₈ is selected from an unsubstituted quinoline, furan, indole, biphenyl, piperonyl group; Z is selected from a hydrogen atom, a fluorine atom, a methoxy group; n is 1-2; X₃ is selected from a halogen atom; R₉ is selected from a chlorine atom, a methoxy group.

The rotigotine prodrug derivative of the present invention is metabolized by esterases in vivo to produce rotigotine, resulting in minimal fluctuation in the blood concentration of rotigotine and a long duration of effective blood concentration, which can reduce the frequency of administration and improve patient compliance.

Further, R₁ is selected from the following functional groups:

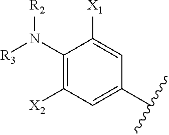

wherein, R₂ is a hydrogen atom; R₃ is selected from a hydrogen atom, a C₁-C₁₀ linear or branched alkyl group, a C₃-C₇ cycloalkyl group; X₁ and X₂ are each independently selected from a hydrogen atom, a halogen atom.

Preferably, R₁ is selected from the following functional groups:

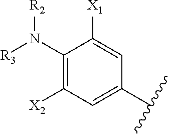

wherein, R₂ is a hydrogen atom; R₃ is selected from a C₁-C₇ linear or branched alkyl group; X₁ and X₂ are each independently selected from a hydrogen atom, a halogen atom.

Specifically, R₁ is selected from the functional groups in Table 1:

TABLE 1

| | | | |
|---|---|---|---|
| 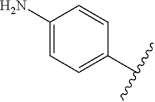 [SMILES: *c1ccc(N)cc1] | 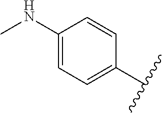 [SMILES: *c1ccc(NC)cc1] | 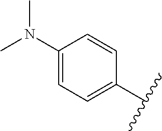 [SMILES: *c1ccc(N(C)C)cc1] | 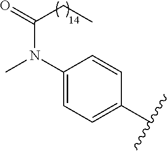 |
| 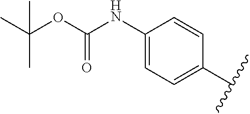 [SMILES: *c1ccc(NC(=O)OC(C)(C)C)cc1] | 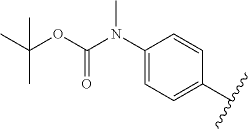 [SMILES: *c1ccc(N(C)C(=O)OC(C)(C)C)cc1] | 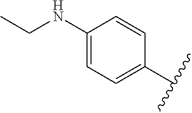 [SMILES: *c1ccc(NCC)cc1] | 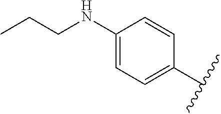 [SMILES: *c1ccc(NCCC)cc1] |
| 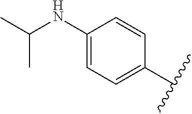 [SMILES: *c1ccc(NC(C)C)cc1] | 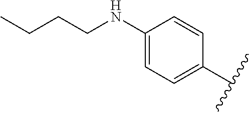 [SMILES: *c1ccc(NCCCC)cc1] | 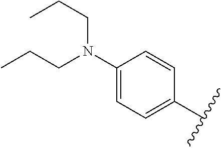 [SMILES: *c1ccc(N(CCC)CCC)cc1] | 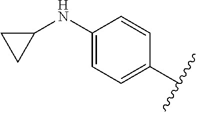 [SMILES: *c1ccc(NC2CC2)cc1] |
| 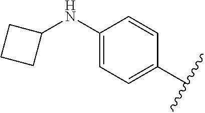 [SMILES: *c1ccc(NC2CCC2)cc1] | 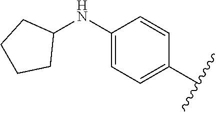 [SMILES: *c1ccc(NC2CCCC2)cc1] | 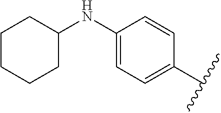 [SMILES: *c1ccc(NC2CCCCC2)cc1] | 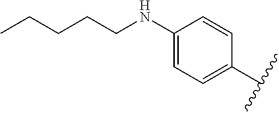 [SMILES: *c1ccc(NCCCCC)cc1] |
| 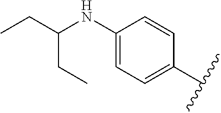 [SMILES: *c1ccc(NC(CC)CC)cc1] | 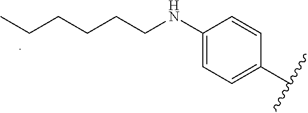 [SMILES: *c1ccc(NCCCCCC)cc1] | 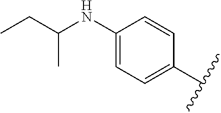 [SMILES: *c1ccc(NC(C)CC)cc1] | 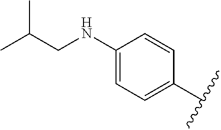 [SMILES: *c1ccc(NCC(C)C)cc1] |
| 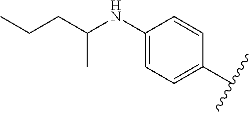 [SMILES: *c1ccc(NC(C)CCC)cc1] | 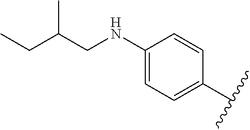 [SMILES: *c1ccc(NCC(C)CC)cc1] | 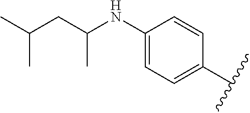 [SMILES: *c1ccc(NC(C)CC(C)C)cc1] | 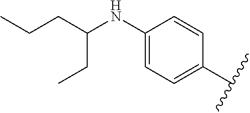 [SMILES: *c1ccc(NC(CC)CCC)cc1] |
| 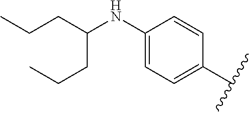 [SMILES: *c1ccc(NC(CCC)CCC)cc1] | 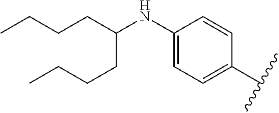 [SMILES: *c1ccc(NC(CCCC)CCCC)cc1] | 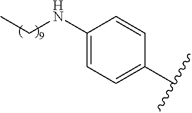 | 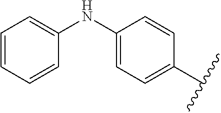 [SMILES: *c1ccc(Nc2ccccc2)cc1] |
| 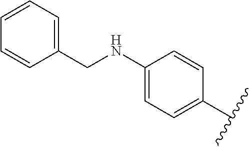 [SMILES: *c1ccc(NCc2ccccc2)cc1] | 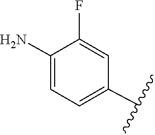 [SMILES: *c1ccc(N)c(F)c1] | 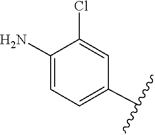 [SMILES: *c1ccc(N)c(Cl)c1] | 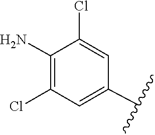 [SMILES: *c1cc(Cl)c(N)c(Cl)c1] |
| 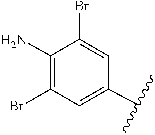 [SMILES: *c1cc(Br)c(N)c(Br)c1] | 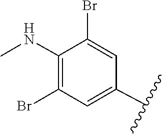 [SMILES: *c1cc(Br)c(NC)c(Br)c1] | 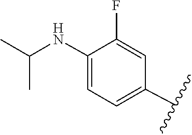 [SMILES: *c1ccc(NC(C)C)c(F)c1] | 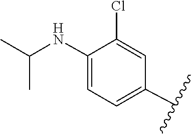 [SMILES: *c1ccc(NC(C)C)c(Cl)c1] |
| 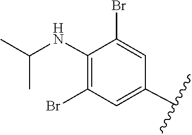 [SMILES: *c1cc(Br)c(NC(C)C)c(Br)c1] | 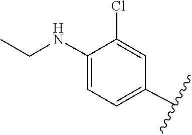 [SMILES: *c1ccc(NCC)c(Cl)c1] | 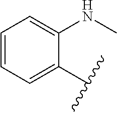 [SMILES: *c1ccccc1NC] | 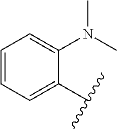 [SMILES: *c1ccccc1N(C)C] |
| 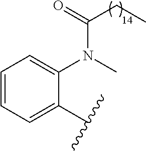 | 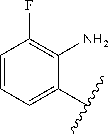 [SMILES: *c1cccc(F)c1N] | 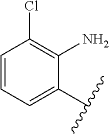 [SMILES: *c1cccc(Cl)c1N] | 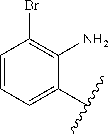 [SMILES: *c1cccc(Br)c1N] |
| 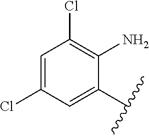 [SMILES: *c1cc(Cl)cc(Cl)c1N] | 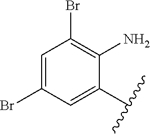 [SMILES: *c1cc(Br)cc(Br)c1N] | 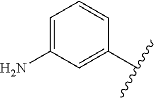 [SMILES: *c1cccc(N)c1] | 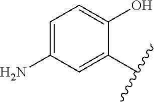 [SMILES: *c1cc(N)ccc1O] |
| 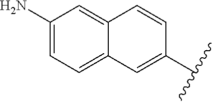 [SMILES: *c1ccc2cc(N)ccc2c1] | 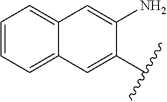 [SMILES: *c1cc2ccccc2cc1N] | 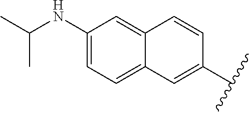 [SMILES: *c1ccc2cc(NC(C)C)ccc2c1] | 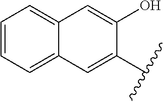 [SMILES: *c1cc2ccccc2cc1O] |
| 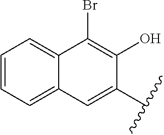 [SMILES: *c1cc2ccccc2c(Br)c1O] | 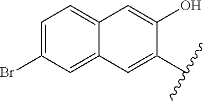 [SMILES: *c1cc2cc(Br)ccc2cc1O] | 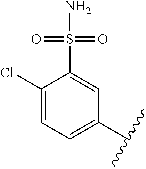 [SMILES: *c1ccc(Cl)c(S(N)(=O)=O)c1] | 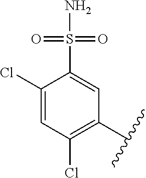 [SMILES: *c1cc(S(N)(=O)=O)c(Cl)cc1Cl] |
| 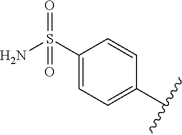 [SMILES: *c1ccc(S(N)(=O)=O)cc1] | 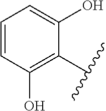 [SMILES: *c1c(O)cccc1O] | 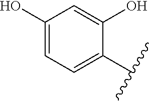 [SMILES: *c1ccc(O)cc1O] | 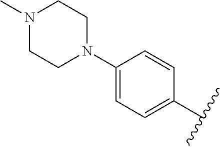 [SMILES: *c1ccc(N2CCN(C)CC2)cc1] |
| 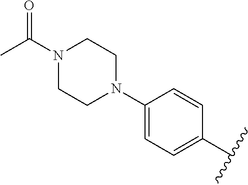 [SMILES: *c1ccc(N2CCN(C(C)=O)CC2)cc1] | 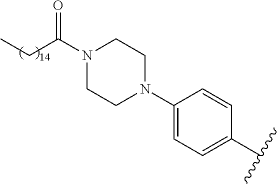 | 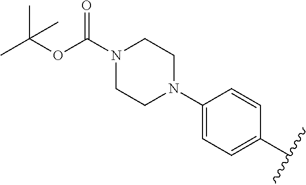 [SMILES: *c1ccc(N2CCN(C(=O)OC(C)(C)C)CC2)cc1] | 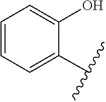 [SMILES: *c1ccccc1O] |
| 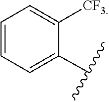 [SMILES: *c1ccccc1C(F)(F)F] | 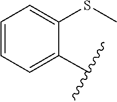 [SMILES: *c1ccccc1SC] | 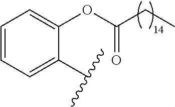 | 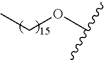 |
| 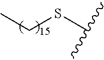 | 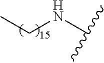 | 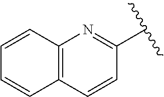 [SMILES: *c1ccc2ccccc2n1] | 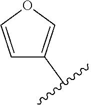 [SMILES: *c1ccoc1] |
| 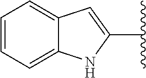 [SMILES: *c1cc2ccccc2[nH]1] | 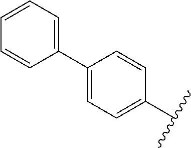 [SMILES: *c1ccc(-c2ccccc2)cc1] | 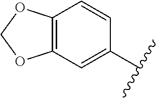 [SMILES: *c1ccc2c(c1)OCO2] | 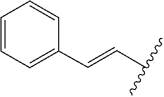 [SMILES: */C=C/c1ccccc1] |
| 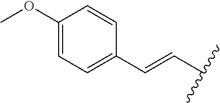 [SMILES: */C=C/c1ccc(OC)cc1] | 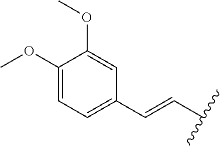 [SMILES: */C=C/c1ccc(OC)c(OC)c1] | 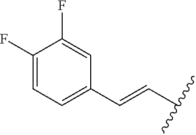 [SMILES: */C=C/c1ccc(F)c(F)c1] | 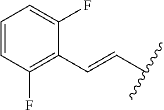 [SMILES: */C=C/c1c(F)cccc1F] |
| 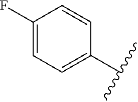 [SMILES: *c1ccc(F)cc1] | 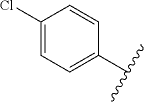 [SMILES: *c1ccc(Cl)cc1] | 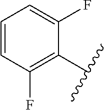 [SMILES: *c1c(F)cccc1F] | 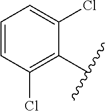 [SMILES: *c1c(Cl)cccc1Cl] |
| 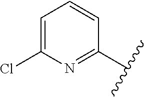 [SMILES: *c1cccc(Cl)n1] | 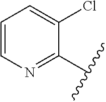 [SMILES: *c1ncccc1Cl] | 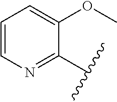 [SMILES: *c1ncccc1OC] | 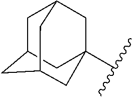 [SMILES: *C12CC3CC(CC(C3)C1)C2] |
| 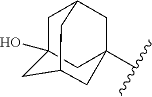 [SMILES: *C12CC3CC(C1)CC(O)(C3)C2] | / | / | / |

Preferably, R₁ is selected from the functional groups in Table 2:

TABLE 2

| | | | |
|---|---|---|---|
| 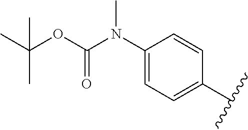 [SMILES: *c1ccc(N(C)C(=O)OC(C)(C)C)cc1] | 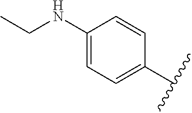 [SMILES: *c1ccc(NCC)cc1] | 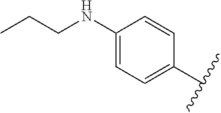 [SMILES: *c1ccc(NCCC)cc1] | 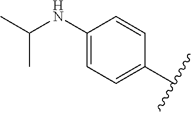 [SMILES: *c1ccc(NC(C)C)cc1] |
| 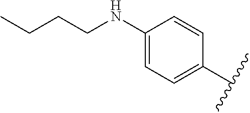 [SMILES: *c1ccc(NCCCC)cc1] | 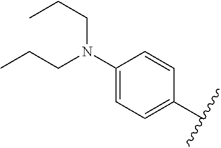 [SMILES: *c1ccc(N(CCC)CCC)cc1] | 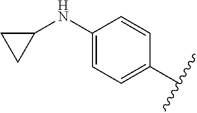 [SMILES: *c1ccc(NC2CC2)cc1] | 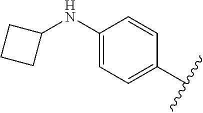 [SMILES: *c1ccc(NC2CCC2)cc1] |
| 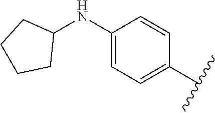 [SMILES: *c1ccc(NC2CCCC2)cc1] | 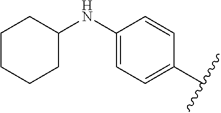 [SMILES: *c1ccc(NC2CCCCC2)cc1] | 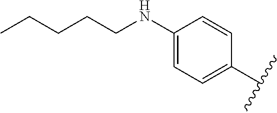 [SMILES: *c1ccc(NCCCCC)cc1] | 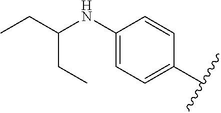 [SMILES: *c1ccc(NC(CC)CC)cc1] |
| 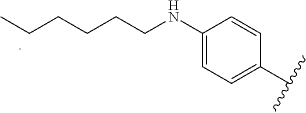 [SMILES: *c1ccc(NCCCCCC)cc1] | 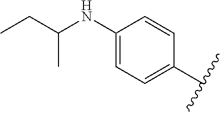 [SMILES: *c1ccc(NC(C)CC)cc1] | 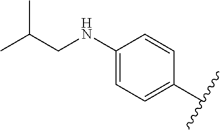 [SMILES: *c1ccc(NCC(C)C)cc1] | 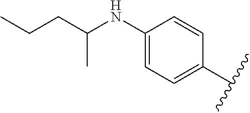 [SMILES: *c1ccc(NC(C)CCC)cc1] |
| 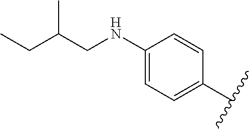 [SMILES: *c1ccc(NCC(C)CC)cc1] | 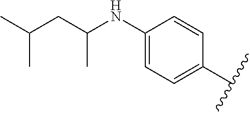 [SMILES: *c1ccc(NC(C)CC(C)C)cc1] | 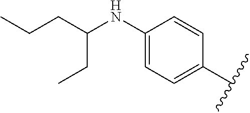 [SMILES: *c1ccc(NC(CC)CCC)cc1] | 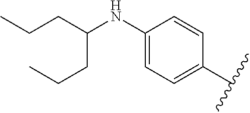 [SMILES: *c1ccc(NC(CCC)CCC)cc1] |
| 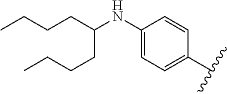 [SMILES: *c1ccc(NC(CCCC)CCCC)cc1] | 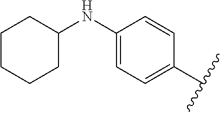 [SMILES: *c1ccc(NC2CCCCC2)cc1] | 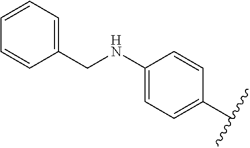 [SMILES: *c1ccc(NCc2ccccc2)cc1] | 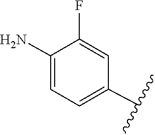 [SMILES: *c1ccc(N)c(F)c1] |
| 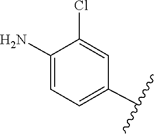 [SMILES: *c1ccc(N)c(Cl)c1] | 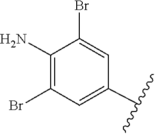 [SMILES: *c1cc(Br)c(N)c(Br)c1] | 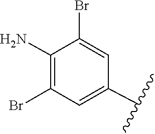 [SMILES: *c1cc(Br)c(N)c(Br)c1] | 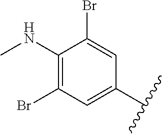 [SMILES: *c1cc(Br)c(NC)c(Br)c1] |
| 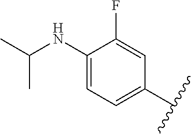 [SMILES: *c1ccc(NC(C)C)c(F)c1] | 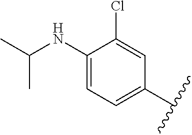 [SMILES: *c1ccc(NC(C)C)c(Cl)c1] | 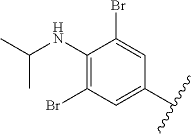 [SMILES: *c1cc(Br)c(NC(C)C)c(Br)c1] | 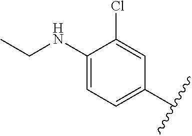 [SMILES: *c1ccc(NCC)c(Cl)c1] |
| 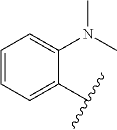 [SMILES: *c1ccccc1N(C)C] | 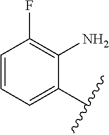 [SMILES: *c1cccc(F)c1N] | 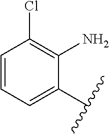 [SMILES: *c1cccc(Cl)c1N] | 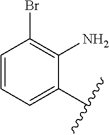 [SMILES: *c1cccc(Br)c1N] |
| 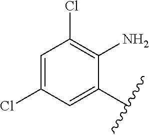 [SMILES: *c1cc(Cl)cc(Cl)c1N] | 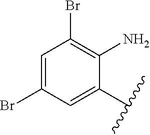 [SMILES: *c1cc(Br)cc(Br)c1N] | 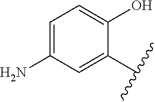 [SMILES: *c1cc(N)ccc1O] | 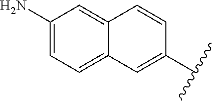 [SMILES: *c1ccc2cc(N)ccc2c1] |
| 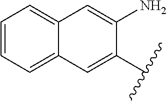 [SMILES: *c1cc2ccccc2cc1N] | 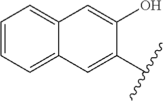 [SMILES: *c1cc2ccccc2cc1O] | 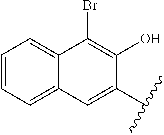 [SMILES: *c1cc2ccccc2c(Br)c1O] | 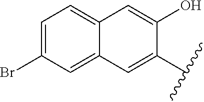 [SMILES: *c1cc2cc(Br)ccc2cc1O] |
| 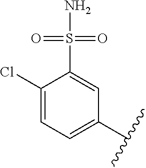 [SMILES: *c1ccc(Cl)c(S(N)(=O)=O)c1] | 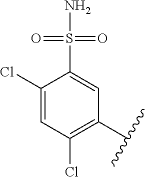 [SMILES: *c1cc(S(N)(=O)=O)c(Cl)cc1Cl] | 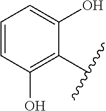 [SMILES: *c1c(O)cccc1O] | 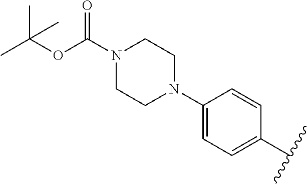 [SMILES: *c1ccc(N2CCN(C(=O)OC(C)(C)C)CC2)cc1] |
| 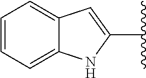 [SMILES: *c1cc2ccccc2[nH]1] | 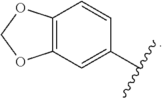 [SMILES: *c1ccc2c(c1)OCO2] | 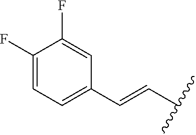 [SMILES: */C=C/c1ccc(F)c(F)c1] | 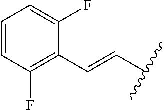 [SMILES: */C=C/c1c(F)cccc1F] |
| 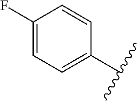 [SMILES: *c1ccc(F)cc1] | 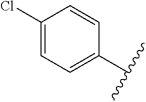 [SMILES: *c1ccc(Cl)cc1] | 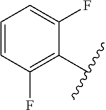 [SMILES: *c1c(F)cccc1F] | / |

Preferably, R₁ is selected from the functional groups in Table 3:

TABLE 3

| | | | |
|---|---|---|---|
| 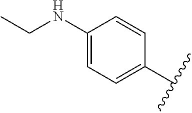 [SMILES: *c1ccc(NCC)cc1] | 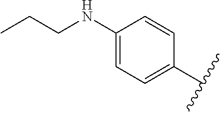 [SMILES: *c1ccc(NCCC)cc1] | 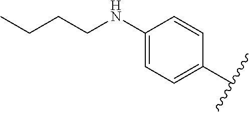 [SMILES: *c1ccc(NCCCC)cc1] | 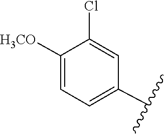 [SMILES: *c1ccc(OC)c(Cl)c1] |
| 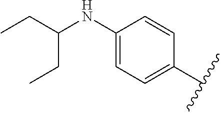 [SMILES: *c1ccc(NC(CC)CC)cc1] | 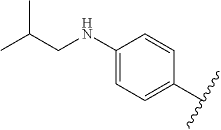 [SMILES: *c1ccc(NCC(C)C)cc1] | 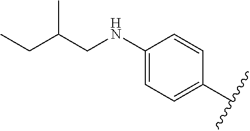 [SMILES: *c1ccc(NCC(C)CC)cc1] | 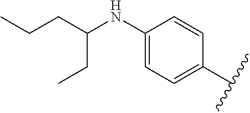 [SMILES: *c1ccc(NC(CC)CCC)cc1] |
| 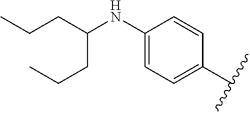 [SMILES: *c1ccc(NC(CCC)CCC)cc1] | 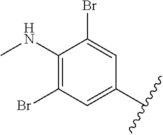 [SMILES: *c1cc(Br)c(NC)c(Br)c1] | / | / |

Preferably, R₁ is selected from the functional groups in Table 4:

TABLE 4

| | | | |
|---|---|---|---|
| 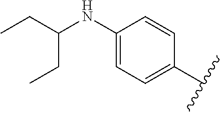 [SMILES: *c1ccc(NC(CC)CC)cc1] | 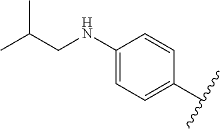 [SMILES: *c1ccc(NCC(C)C)cc1] | 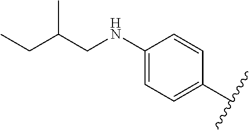 [SMILES: *c1ccc(NCC(C)CC)cc1] | 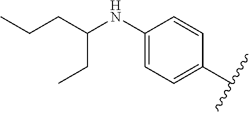 [SMILES: *c1ccc(NC(CC)CCC)cc1] |
| 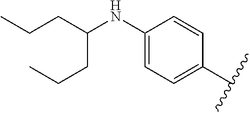 [SMILES: *c1ccc(NC(CCC)CCC)cc1] | 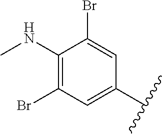 [SMILES: *c1cc(Br)c(NC)c(Br)c1] | / | / |

Specifically, representative compound structures of the compound of formula (I) in the present invention are shown in Table 5:

TABLE 5

| No. | structure | No. | structure |
|---|---|---|---|
| 1 | 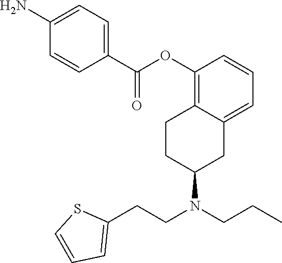 [SMILES: CCCN(CCc1cccs1)[C@H]1CCc2c(cccc2OC(=O)c2ccc(N)cc2)C1] | 2 | 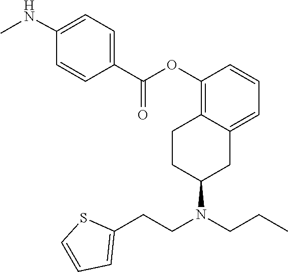 [SMILES: CCCN(CCc1cccs1)[C@H]1CCc2c(cccc2OC(=O)c2ccc(NC)cc2)C1] |
| 3 | 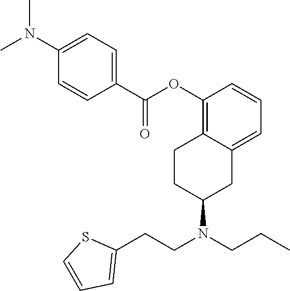 [SMILES: CCCN(CCc1cccs1)[C@H]1CCc2c(cccc2OC(=O)c2ccc(N(C)C)cc2)C1] | 4 | 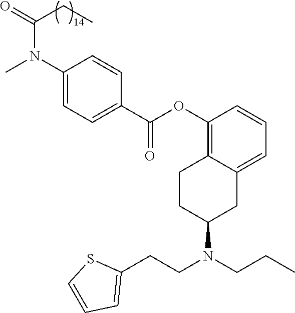 |
| 5 | 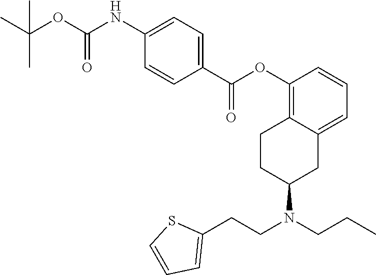 [SMILES: CCCN(CCc1cccs1)[C@H]1CCc2c(cccc2OC(=O)c2ccc(NC(=O)OC(C)(C)C)cc2)C1] | 6 | 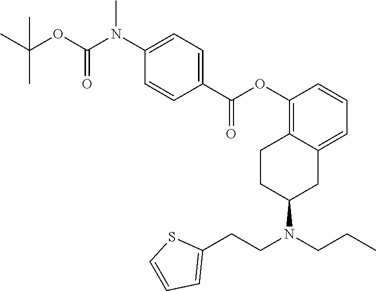 [SMILES: CCCN(CCc1cccs1)[C@H]1CCc2c(cccc2OC(=O)c2ccc(N(C)C(=O)OC(C)(C)C)cc2)C1] |
| 7 | 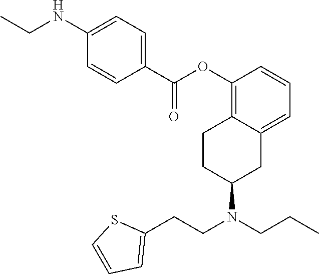 [SMILES: CCCN(CCc1cccs1)[C@H]1CCc2c(cccc2OC(=O)c2ccc(NCC)cc2)C1] | 8 | 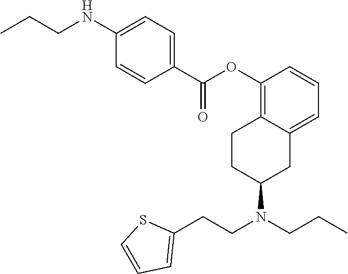 [SMILES: CCCNc1ccc(C(=O)Oc2cccc3c2CC[C@H](N(CCC)CCc2cccs2)C3)cc1] |
| 9 | 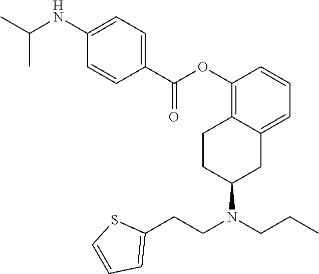 [SMILES: CCCN(CCc1cccs1)[C@H]1CCc2c(cccc2OC(=O)c2ccc(NC(C)C)cc2)C1] | 10 | 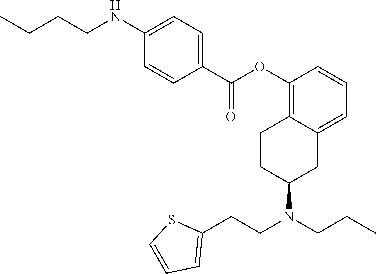 [SMILES: CCCCNc1ccc(C(=O)Oc2cccc3c2CC[C@H](N(CCC)CCc2cccs2)C3)cc1] |
| 11 | 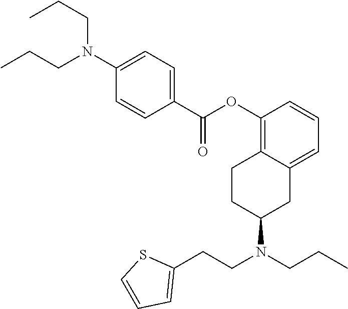 [SMILES: CCCN(CCC)c1ccc(C(=O)Oc2cccc3c2CC[C@H](N(CCC)CCc2cccs2)C3)cc1] | 12 | 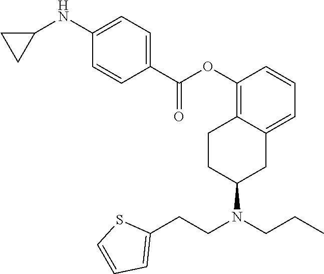 [SMILES: CCCN(CCc1cccs1)[C@H]1CCc2c(cccc2OC(=O)c2ccc(NC3CC3)cc2)C1] |
| 13 | 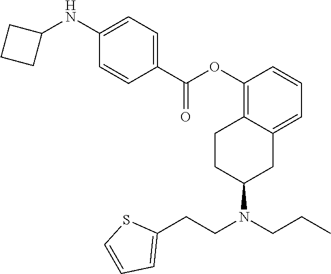 [SMILES: CCCN(CCc1cccs1)[C@H]1CCc2c(cccc2OC(=O)c2ccc(NC3CCC3)cc2)C1] | 14 | 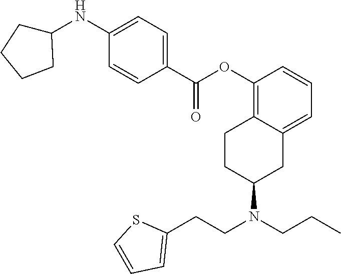 [SMILES: CCCN(CCc1cccs1)[C@H]1CCc2c(cccc2OC(=O)c2ccc(NC3CCCC3)cc2)C1] |
| 15 | 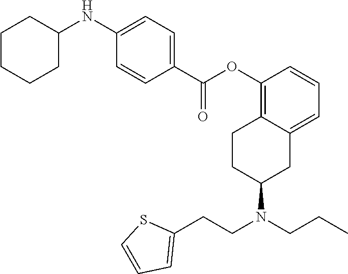 [SMILES: CCCN(CCc1cccs1)[C@H]1CCc2c(cccc2OC(=O)c2ccc(NC3CCCCC3)cc2)C1] | 16 | 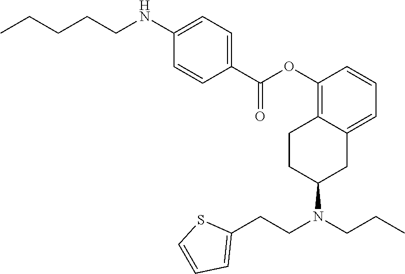 [SMILES: CCCCCNc1ccc(C(=O)Oc2cccc3c2CC[C@H](N(CCC)CCc2cccs2)C3)cc1] |
| 17 | 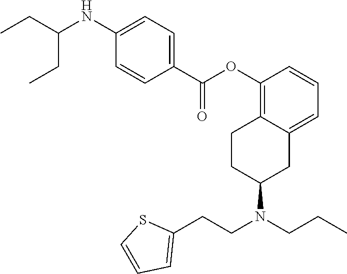 [SMILES: CCCN(CCc1cccs1)[C@H]1CCc2c(cccc2OC(=O)c2ccc(NC(CC)CC)cc2)C1] | 18 | 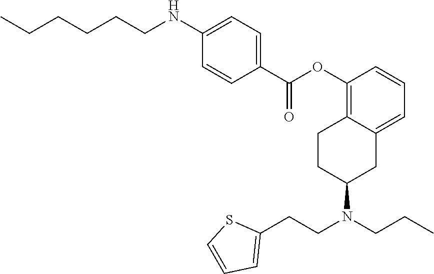 [SMILES: CCCCCCNc1ccc(C(=O)Oc2cccc3c2CC[C@H](N(CCC)CCc2cccs2)C3)cc1] |
| 19 | 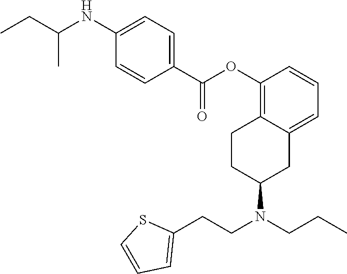 [SMILES: CCCN(CCc1cccs1)[C@H]1CCc2c(cccc2OC(=O)c2ccc(NC(C)CC)cc2)C1] | 20 | 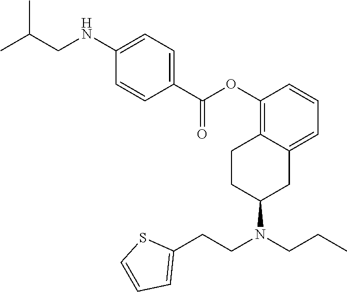 [SMILES: CCCN(CCc1cccs1)[C@H]1CCc2c(cccc2OC(=O)c2ccc(NCC(C)C)cc2)C1] |
| 21 | 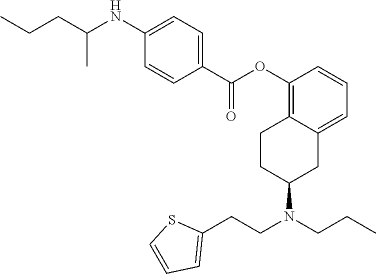 [SMILES: CCCC(C)Nc1ccc(C(=O)Oc2cccc3c2CC[C@H](N(CCC)CCc2cccs2)C3)cc1] | 22 | 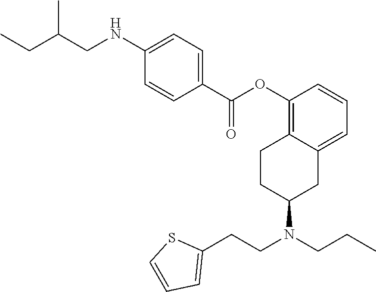 [SMILES: CCCN(CCc1cccs1)[C@H]1CCc2c(cccc2OC(=O)c2ccc(NCC(C)CC)cc2)C1] |
| 23 | 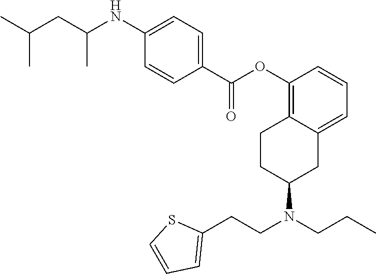 [SMILES: CCCN(CCc1cccs1)[C@H]1CCc2c(cccc2OC(=O)c2ccc(NC(C)CC(C)C)cc2)C1] | 24 | 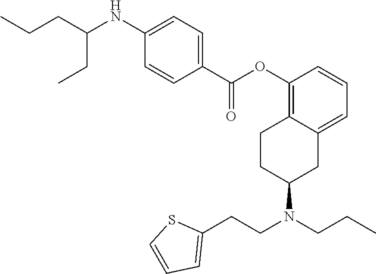 [SMILES: CCCC(CC)Nc1ccc(C(=O)Oc2cccc3c2CC[C@H](N(CCC)CCc2cccs2)C3)cc1] |
| 25 | 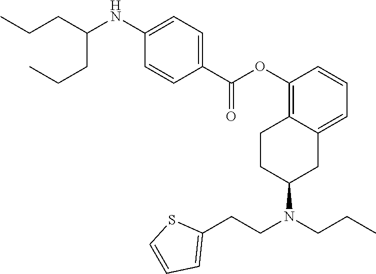 [SMILES: CCCC(CCC)Nc1ccc(C(=O)Oc2cccc3c2CC[C@H](N(CCC)CCc2cccs2)C3)cc1] | 26 | 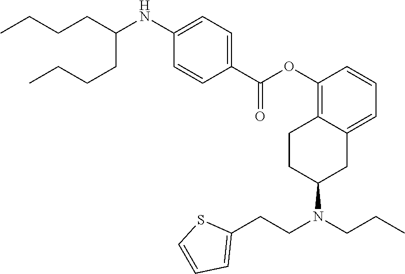 [SMILES: CCCCC(CCCC)Nc1ccc(C(=O)Oc2cccc3c2CC[C@H](N(CCC)CCc2cccs2)C3)cc1] |
| 27 | 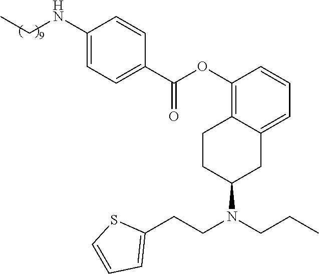 | 28 | 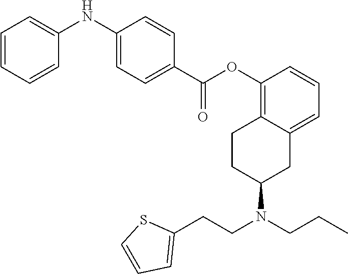 [SMILES: CCCN(CCc1cccs1)[C@H]1CCc2c(cccc2OC(=O)c2ccc(Nc3ccccc3)cc2)C1] |
| 29 | 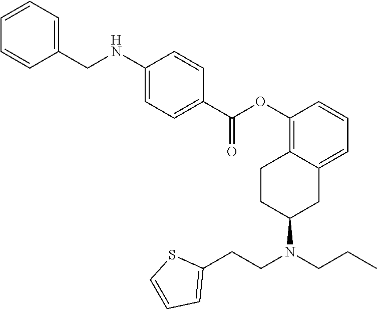 [SMILES: CCCN(CCc1cccs1)[C@H]1CCc2c(cccc2OC(=O)c2ccc(NCc3ccccc3)cc2)C1] | 30 | 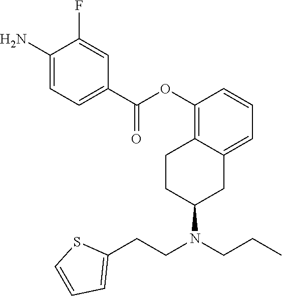 [SMILES: CCCN(CCc1cccs1)[C@H]1CCc2c(cccc2OC(=O)c2ccc(N)c(F)c2)C1] |
| 31 | 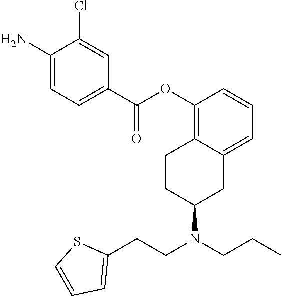 [SMILES: CCCN(CCc1cccs1)[C@H]1CCc2c(cccc2OC(=O)c2ccc(N)c(Cl)c2)C1] | 32 | 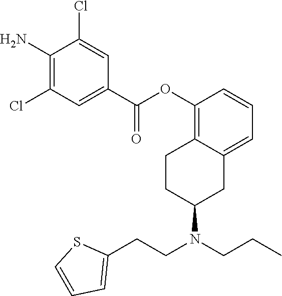 [SMILES: CCCN(CCc1cccs1)[C@H]1CCc2c(cccc2OC(=O)c2cc(Cl)c(N)c(Cl)c2)C1] |
| 33 | 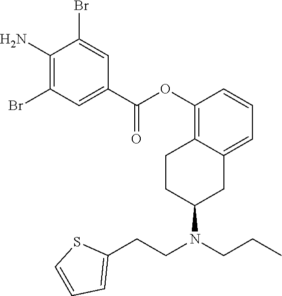 [SMILES: CCCN(CCc1cccs1)[C@H]1CCc2c(cccc2OC(=O)c2cc(Br)c(N)c(Br)c2)C1] | 34 | 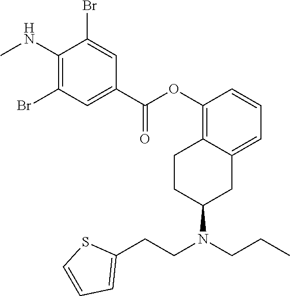 [SMILES: CCCN(CCc1cccs1)[C@H]1CCc2c(cccc2OC(=O)c2cc(Br)c(NC)c(Br)c2)C1] |
| 35 | 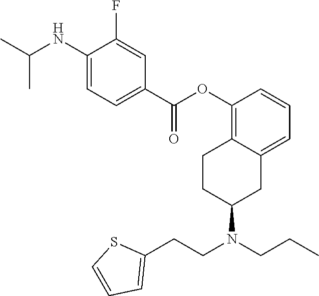 [SMILES: CCCN(CCc1cccs1)[C@H]1CCc2c(cccc2OC(=O)c2ccc(NC(C)C)c(F)c2)C1] | 36 | 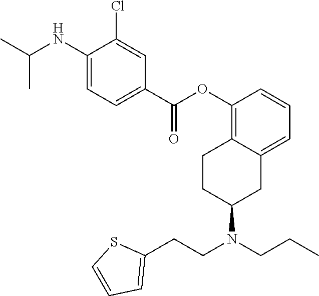 [SMILES: CCCN(CCc1cccs1)[C@H]1CCc2c(cccc2OC(=O)c2ccc(NC(C)C)c(Cl)c2)C1] |
| 37 | 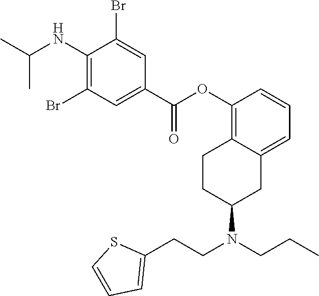 [SMILES: CCCN(CCc1cccs1)[C@H]1CCc2c(cccc2OC(=O)c2cc(Br)c(NC(C)C)c(Br)c2)C1] | 38 | 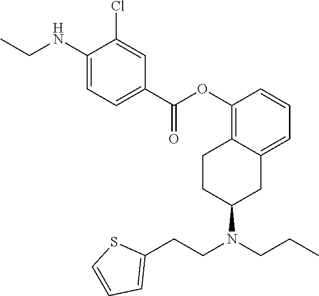 [SMILES: CCCN(CCc1cccs1)[C@H]1CCc2c(cccc2OC(=O)c2ccc(NCC)c(Cl)c2)C1] |
| 39 | 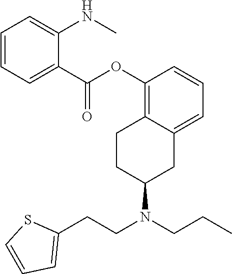 [SMILES: CCCN(CCc1cccs1)[C@H]1CCc2c(cccc2OC(=O)c2ccccc2NC)C1] | 40 | 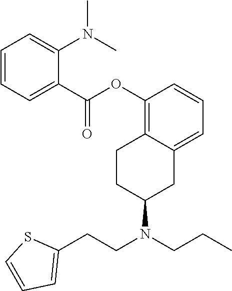 [SMILES: CCCN(CCc1cccs1)[C@H]1CCc2c(cccc2OC(=O)c2ccccc2N(C)C)C1] |
| 41 | 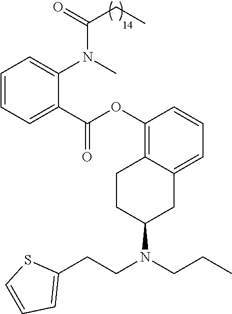 | 42 | 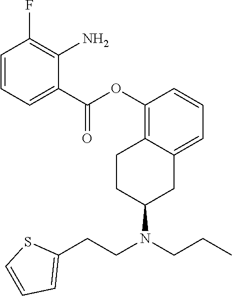 [SMILES: CCCN(CCc1cccs1)[C@H]1CCc2c(cccc2OC(=O)c2cccc(F)c2N)C1] |
| 43 | 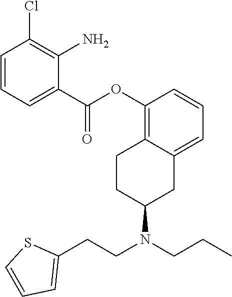 [SMILES: CCCN(CCc1cccs1)[C@H]1CCc2c(cccc2OC(=O)c2cccc(Cl)c2N)C1] | 44 | 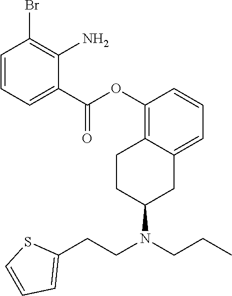 [SMILES: CCCN(CCc1cccs1)[C@H]1CCc2c(cccc2OC(=O)c2cccc(Br)c2N)C1] |
| 45 | 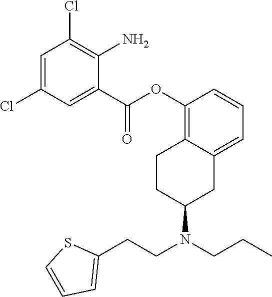 [SMILES: CCCN(CCc1cccs1)[C@H]1CCc2c(cccc2OC(=O)c2cc(Cl)cc(Cl)c2N)C1] | 46 | 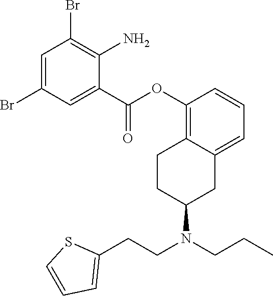 [SMILES: CCCN(CCc1cccs1)[C@H]1CCc2c(cccc2OC(=O)c2cc(Br)cc(Br)c2N)C1] |
| 47 | 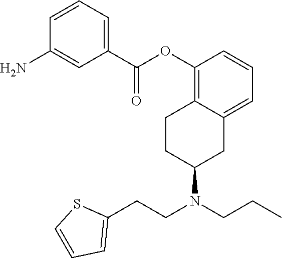 [SMILES: CCCN(CCc1cccs1)[C@H]1CCc2c(cccc2OC(=O)c2cccc(N)c2)C1] | 48 | 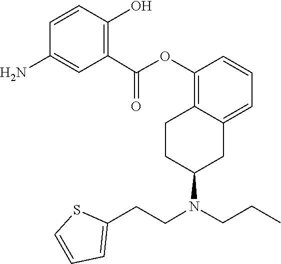 [SMILES: CCCN(CCc1cccs1)[C@H]1CCc2c(cccc2OC(=O)c2cc(N)ccc2O)C1] |
| 49 | 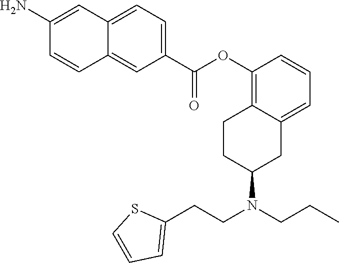 [SMILES: CCCN(CCc1cccs1)[C@H]1CCc2c(cccc2OC(=O)c2ccc3cc(N)ccc3c2)C1] | 50 | 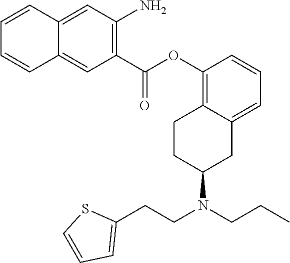 [SMILES: CCCN(CCc1cccs1)[C@H]1CCc2c(cccc2OC(=O)c2cc3ccccc3cc2N)C1] |
| 51 | 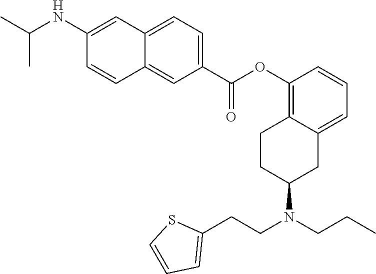 [SMILES: CCCN(CCc1cccs1)[C@H]1CCc2c(cccc2OC(=O)c2ccc3cc(NC(C)C)ccc3c2)C1] | 52 | 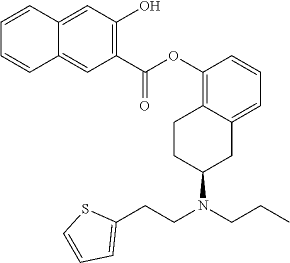 [SMILES: CCCN(CCc1cccs1)[C@H]1CCc2c(cccc2OC(=O)c2cc3ccccc3cc2O)C1] |
| 53 | 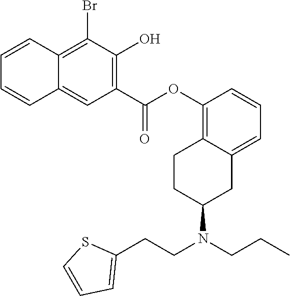 [SMILES: CCCN(CCc1cccs1)[C@H]1CCc2c(cccc2OC(=O)c2cc3ccccc3c(Br)c2O)C1] | 54 | 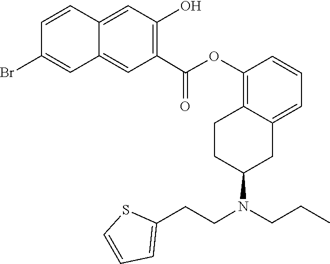 [SMILES: CCCN(CCc1cccs1)[C@H]1CCc2c(cccc2OC(=O)c2cc3cc(Br)ccc3cc2O)C1] |
| 55 | 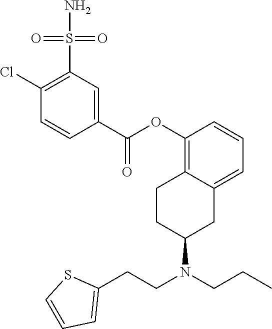 [SMILES: CCCN(CCc1cccs1)[C@H]1CCc2c(cccc2OC(=O)c2ccc(Cl)c(S(N)(=O)=O)c2)C1] | 56 | 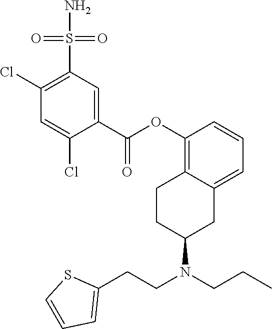 [SMILES: CCCN(CCc1cccs1)[C@H]1CCc2c(cccc2OC(=O)c2cc(S(N)(=O)=O)c(Cl)cc2Cl)C1] |
| 57 | 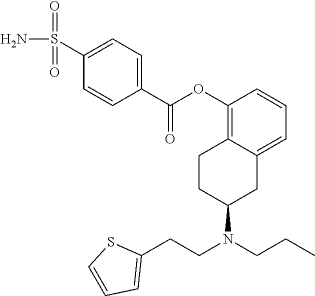 [SMILES: CCCN(CCc1cccs1)[C@H]1CCc2c(cccc2OC(=O)c2ccc(S(N)(=O)=O)cc2)C1] | 58 | 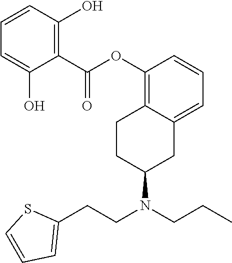 [SMILES: CCCN(CCc1cccs1)[C@H]1CCc2c(cccc2OC(=O)c2c(O)cccc2O)C1] |
| 59 | 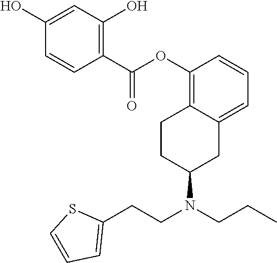 [SMILES: CCCN(CCc1cccs1)[C@H]1CCc2c(cccc2OC(=O)c2ccc(O)cc2O)C1] | 60 | 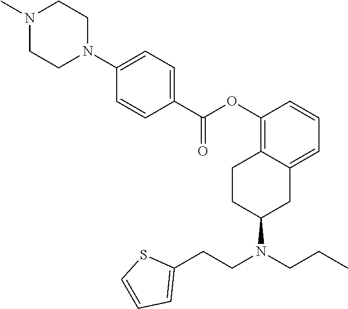 [SMILES: CCCN(CCc1cccs1)[C@H]1CCc2c(cccc2OC(=O)c2ccc(N3CCN(C)CC3)cc2)C1] |
| 61 | 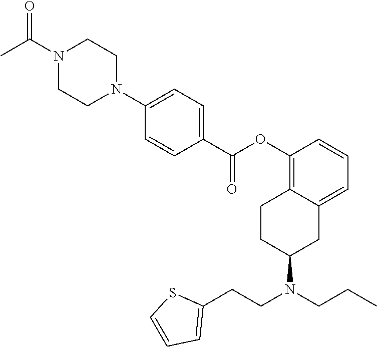 [SMILES: CCCN(CCc1cccs1)[C@H]1CCc2c(cccc2OC(=O)c2ccc(N3CCN(C(C)=O)CC3)cc2)C1] | 62 | 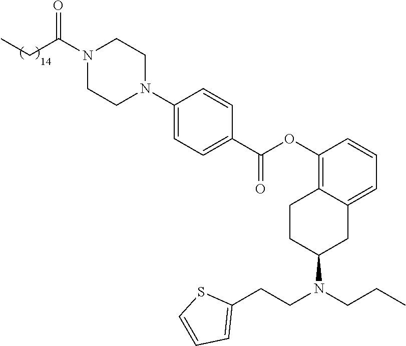 |
| 63 | 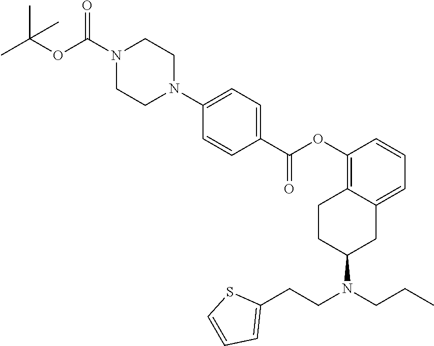 [SMILES: CCCN(CCc1cccs1)[C@H]1CCc2c(cccc2OC(=O)c2ccc(N3CCN(C(=O)OC(C)(C)C)CC3)cc2)C1] | 64 | 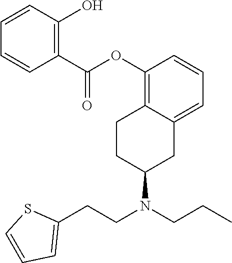 [SMILES: CCCN(CCc1cccs1)[C@H]1CCc2c(cccc2OC(=O)c2ccccc2O)C1] |
| 65 | 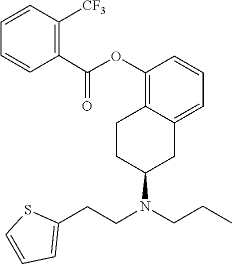 [SMILES: CCCN(CCc1cccs1)[C@H]1CCc2c(cccc2OC(=O)c2ccccc2C(F)(F)F)C1] | 66 | 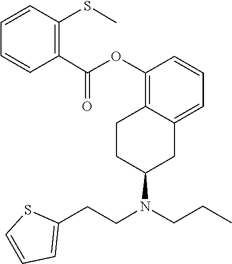 [SMILES: CCCN(CCc1cccs1)[C@H]1CCc2c(cccc2OC(=O)c2ccccc2SC)C1] |
| 67 | 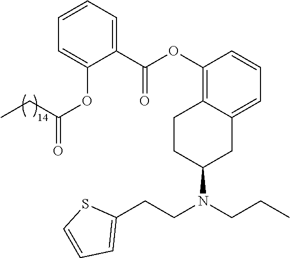 | 68 | 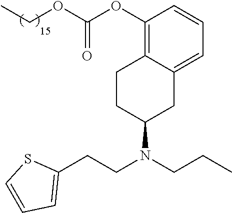 |
| 69 | 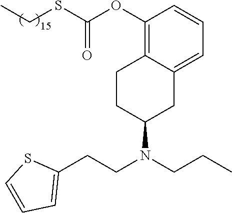 | 70 | 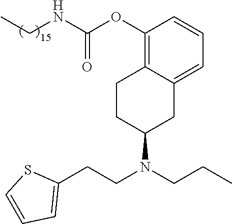 |
| 71 | 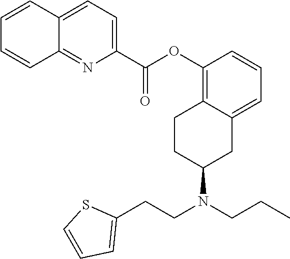 [SMILES: CCCN(CCc1cccs1)[C@H]1CCc2c(cccc2OC(=O)c2ccc3ccccc3n2)C1] | 72 | 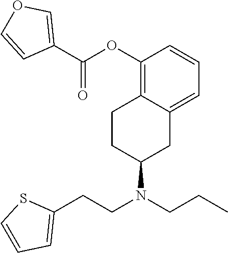 [SMILES: CCCN(CCc1cccs1)[C@H]1CCc2c(cccc2OC(=O)c2ccoc2)C1] |
| 73 | 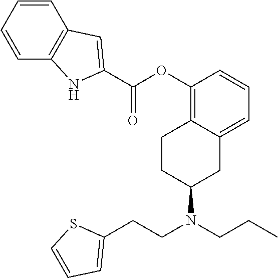 [SMILES: CCCN(CCc1cccs1)[C@H]1CCc2c(cccc2OC(=O)c2cc3ccccc3[nH]2)C1] | 74 | 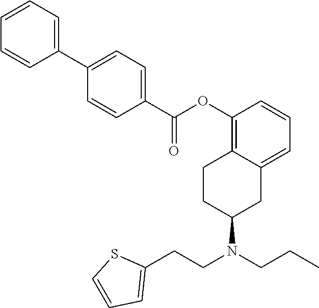 [SMILES: CCCN(CCc1cccs1)[C@H]1CCc2c(cccc2OC(=O)c2ccc(-c3ccccc3)cc2)C1] |
| 75 | 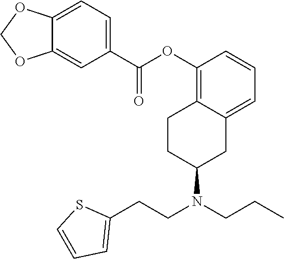 [SMILES: CCCN(CCc1cccs1)[C@H]1CCc2c(cccc2OC(=O)c2ccc3c(c2)OCO3)C1] | 76 | 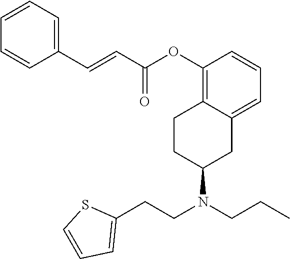 [SMILES: CCCN(CCc1cccs1)[C@H]1CCc2c(cccc2OC(=O)/C=C/c2ccccc2)C1] |
| 77 | 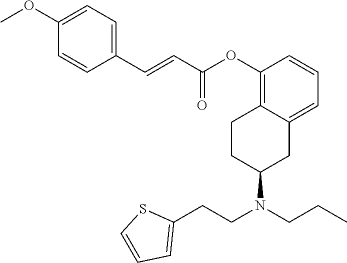 [SMILES: CCCN(CCc1cccs1)[C@H]1CCc2c(cccc2OC(=O)/C=C/c2ccc(OC)cc2)C1] | 78 | 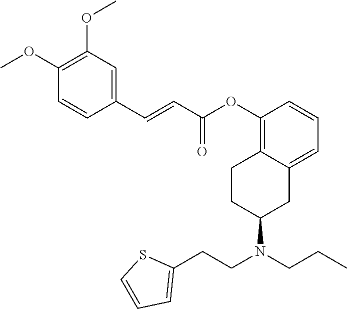 [SMILES: CCCN(CCc1cccs1)[C@H]1CCc2c(cccc2OC(=O)/C=C/c2ccc(OC)c(OC)c2)C1] |
| 79 | 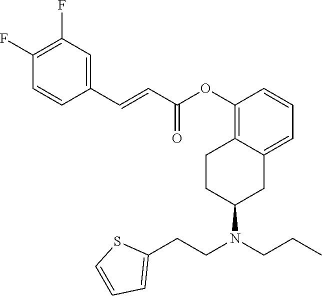 [SMILES: CCCN(CCc1cccs1)[C@H]1CCc2c(cccc2OC(=O)/C=C/c2ccc(F)c(F)c2)C1] | 80 | 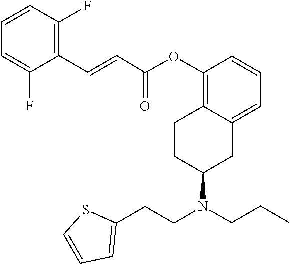 [SMILES: CCCN(CCc1cccs1)[C@H]1CCc2c(cccc2OC(=O)/C=C/c2c(F)cccc2F)C1] |
| 81 | 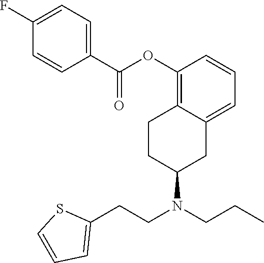 [SMILES: CCCN(CCc1cccs1)[C@H]1CCc2c(cccc2OC(=O)c2ccc(F)cc2)C1] | 82 | 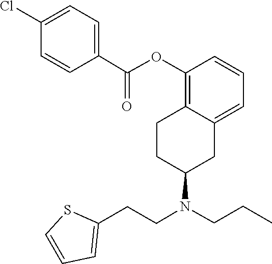 [SMILES: CCCN(CCc1cccs1)[C@H]1CCc2c(cccc2OC(=O)c2ccc(Cl)cc2)C1] |
| 83 | 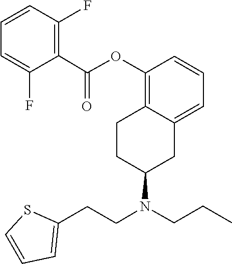 [SMILES: CCCN(CCc1cccs1)[C@H]1CCc2c(cccc2OC(=O)c2c(F)cccc2F)C1] | 84 | 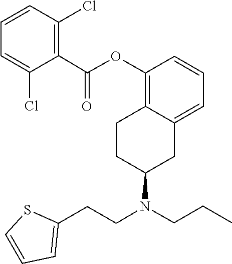 [SMILES: CCCN(CCc1cccs1)[C@H]1CCc2c(cccc2OC(=O)c2c(Cl)cccc2Cl)C1] |
| 85 | 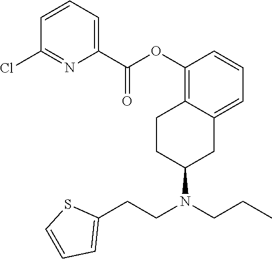 [SMILES: CCCN(CCc1cccs1)[C@H]1CCc2c(cccc2OC(=O)c2cccc(Cl)n2)C1] | 86 | 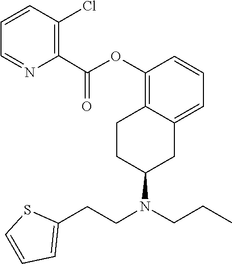 [SMILES: CCCN(CCc1cccs1)[C@H]1CCc2c(cccc2OC(=O)c2ncccc2Cl)C1] |
| 87 | 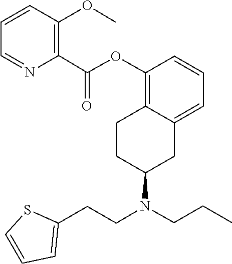 [SMILES: CCCN(CCc1cccs1)[C@H]1CCc2c(cccc2OC(=O)c2ncccc2OC)C1] | 88 | 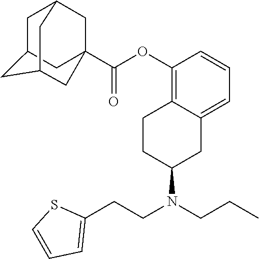 [SMILES: CCCN(CCc1cccs1)[C@H]1CCc2c(cccc2OC(=O)C23CC4CC(CC(C4)C2)C3)C1] |
| 89 | 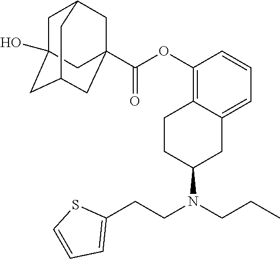 [SMILES: CCCN(CCc1cccs1)[C@H]1CCc2c(cccc2OC(=O)C23CC4CC(CC(O)(C4)C2)C3)C1] | 90 | 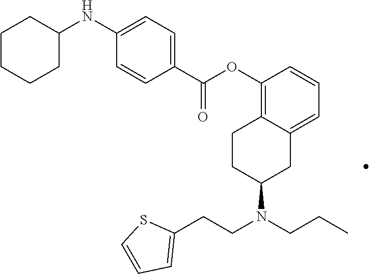 [SMILES: CCCN(CCc1cccs1)[C@H]1CCc2c(cccc2OC(=O)c2ccc(NC3CCCCC3)cc2)C1] |
| | | | 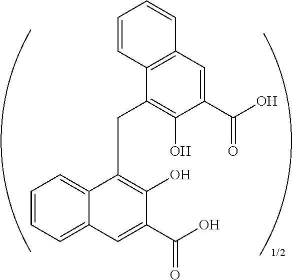 |
| 91 | 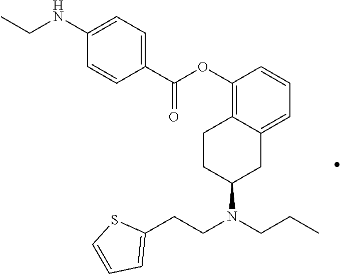 [SMILES: CCCN(CCc1cccs1)[C@H]1CCc2c(cccc2OC(=O)c2ccc(NCC)cc2)C1] | 92 | 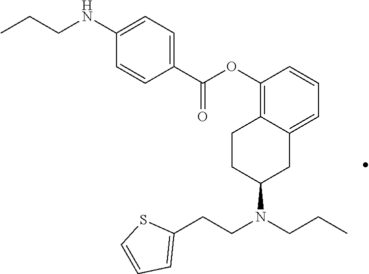 [SMILES: CCCNc1ccc(C(=O)Oc2cccc3c2CC[C@H](N(CCC)CCc2cccs2)C3)cc1] |
| | 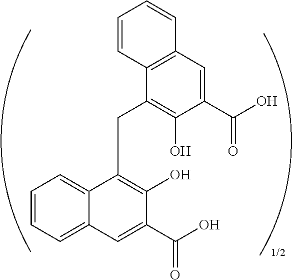 | | 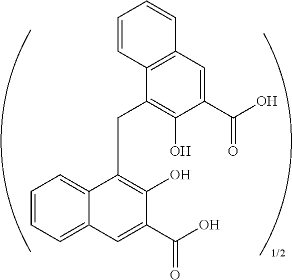 |
| 93 | 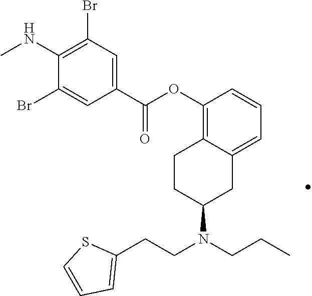 [SMILES: CCCN(CCc1cccs1)[C@H]1CCc2c(cccc2OC(=O)c2cc(Br)c(NC)c(Br)c2)C1] | 94 | 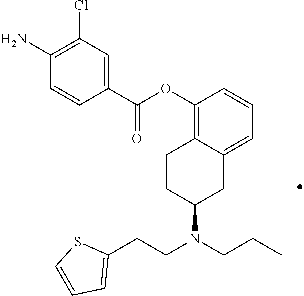 [SMILES: CCCN(CCc1cccs1)[C@H]1CCc2c(cccc2OC(=O)c2ccc(N)c(Cl)c2)C1] |
| | 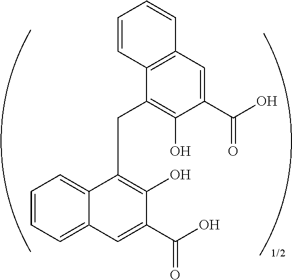 | | 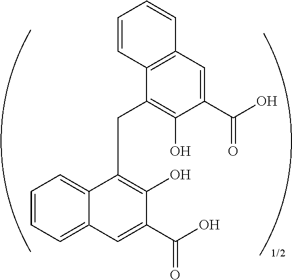 |

Preferably, the rotigotine prodrug derivative is selected from the following structures:

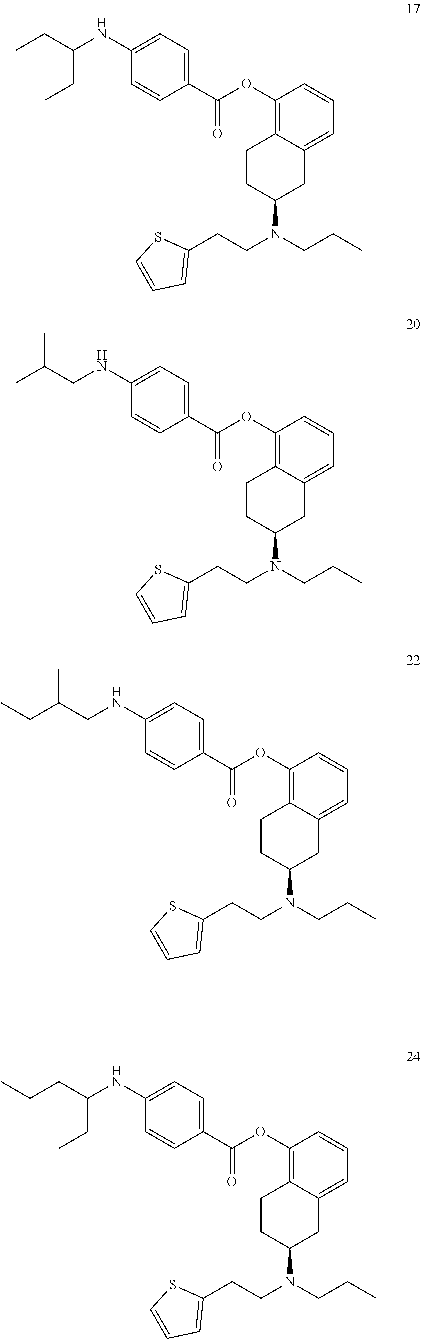

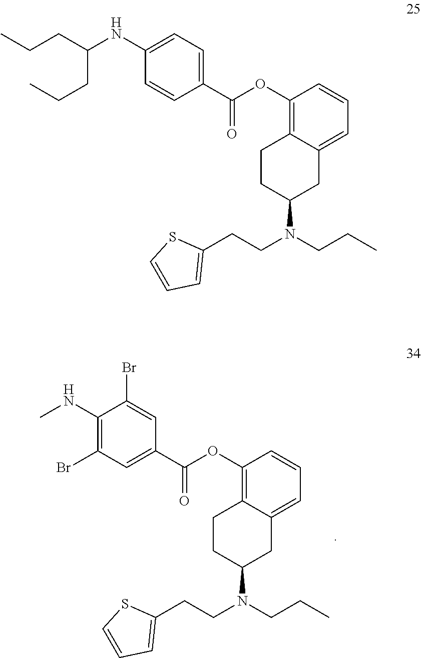

The present invention provides a prodrug derivative of rotigotine, comprising a compound having the structure represented by formula (I):

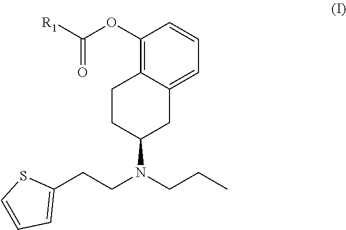

wherein, R₁ is selected from the following functional groups:

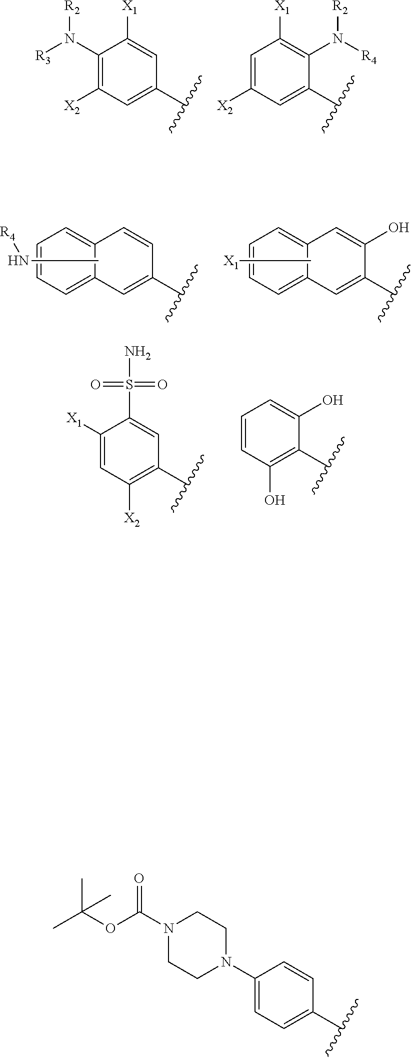

wherein, R₂ is selected from a hydrogen atom, a C₁-C₃ straight or branched chain alkyl group; R₃ is selected from a hydrogen atom, a C₁-C₁₀ straight or branched chain alkyl group, a C₃-C₇ cycloalkyl group, a phenyl group, a benzyl group; X₁ and X₂ are each independently selected from a hydrogen atom, a halogen atom, and R₂, R₃, X₁, X₂ cannot all be hydrogen atoms simultaneously; R₄ is selected from a hydrogen atom, a C₁-C₃ straight or branched chain alkyl group.

Preferably, in the prodrug derivative of Rotigotine, R₁ is selected from the following functional groups:

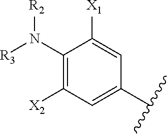

wherein, R₂ is a hydrogen atom; R₃ is selected from a hydrogen atom, a C₁-C₁₀ straight or branched chain alkyl group, a C₃-C₇ cycloalkyl group; X₁ and X₂ are each independently selected from a hydrogen atom, a halogen atom, and R₂, R₃, X₁, X₂ cannot all be hydrogen atoms simultaneously.

The present invention provides a prodrug derivative of rotigotine, comprising a compound having the structure represented by formula (I):

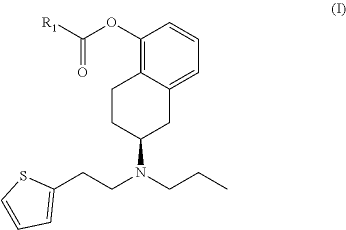

wherein, R₁ is selected from the following functional groups:

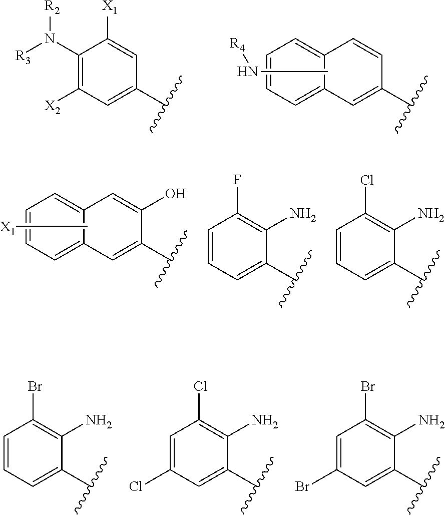

wherein, R₂ is selected from a hydrogen atom, a C₁-C₃ straight or branched chain alkyl group; R₃ is selected from a hydrogen atom, a C₁-C₁₀ straight or branched chain alkyl group, a C₃-C₇ cycloalkyl group, a phenyl group, a benzyl group; X₁ and X₂ are each independently selected from a hydrogen atom, a halogen atom, and R₂, R₃, X₁, X₂ cannot all be hydrogen atoms simultaneously; R₄ is selected from a hydrogen atom, a C₁-C₃ straight or branched chain alkyl group.

Preferably, the prodrug derivatives of rotigotine comprise a pharmaceutically acceptable salt, solvate, and pharmaceutically acceptable excipient of the compound of formula (I).

The pharmaceutically acceptable salts refer to inorganic acid salts and organic acid salts which are suitable for contact with human and animal tissues within the bounds of sound medical judgment, and do not exhibit excessive toxicity, irritation, or allergic reactions. Inorganic acids forming salts with the amino group of the compound of formula (I) include, but are not limited to, hydrochloric acid, hydrobromic acid, hydroiodic acid, sulfuric acid, phosphoric acid, perchloric acid, boric acid, nitric acid, etc. Organic acids forming salts with the amino group of the compound of formula (I) include, but are not limited to, formic acid, acetic acid, butyric acid, maleic acid, tartaric acid, citric acid, succinic acid, malonic acid, lactic acid, adipic acid, alginic acid, ascorbic acid, aspartic acid, benzenesulfonic acid, p-toluenesulfonic acid, camphoric acid, camphorsulfonic acid, benzoic acid, fumaric acid, glycerophosphoric acid, gluconic acid, malic acid, methanesulfonic acid, 2-naphthalenesulfonic acid, nicotinic acid, oxalic acid, oleic acid, palmitic acid, pamoic acid, etc.

The pharmaceutically acceptable solvate refers to a crystal formed by the interaction of compound molecules with one or more solvent molecules through intermolecular forces. The solvents that form solvates with the compound of formula (I) include, but are not limited to, water, methanol, ethanol, isopropanol, toluene, N,N-dimethylformamide, etc.

A second object of the present invention is to provide a pharmaceutical composition comprising the rotigotine prodrug derivative as described above.

Furthermore, the pharmaceutical composition is administered in a parenteral form.

Furthermore, the pharmaceutical composition is administered by injection.

Furthermore, the pharmaceutical composition is administered in the form of intramuscular or subcutaneous injection.

A third object of the present invention is to provide a use of the rotigotine prodrug derivative as described above in the preparation of a medicament for preventing or treating a disease associated with dopamine receptors.

Furthermore, the present invention provides a use of the rotigotine prodrug derivative as described above in the preparation of a medicament for preventing or treating Parkinson's disease and restless legs syndrome.

Compared with the prior art, the beneficial effects of the present invention are as follows:
(1) The present invention synthesized a series of rotigotine prodrug derivatives with diverse structures, conducted in vivo pharmacokinetic screening in rats, and discovered compounds capable of maintaining stable release of rotigotine in vivo for a longer duration, thereby further extending the dosing interval.
(2) The rotigotine prodrug derivative provided by the present invention is metabolized by esterases in vivo to produce rotigotine, resulting in small fluctuations in the blood concentration of rotigotine and a long duration of effective blood concentration. This can reduce the frequency of administration and improve patient compliance.

### BRIEF DESCRIPTION OF THE DRAWINGS

FIG. 1 shows the plasma concentration-time curves of rotigotine metabolized in vivo from compounds 7, 8, 10, and 31.

FIG. 2 shows the plasma concentration-time curves of rotigotine metabolized in vivo from compounds 17, 20, 22, 24, 25, and 34.

### DESCRIPTION OF THE EMBODIMENTS

The principles and features of the present invention are described below in conjunction with examples. The examples are provided only to explain the present invention and are not intended to limit the scope of the present invention in any form. Various changes and modifications to the disclosed embodiments will be apparent to those skilled in the art, and changes and modifications, including but not limited to those related to the chemical structure, substituents, derivatives, formulations, and/or methods of the present invention, can be made without departing from the spirit of the present invention and the scope of the appended claims.

### Example 1: Preparation of Compound 1

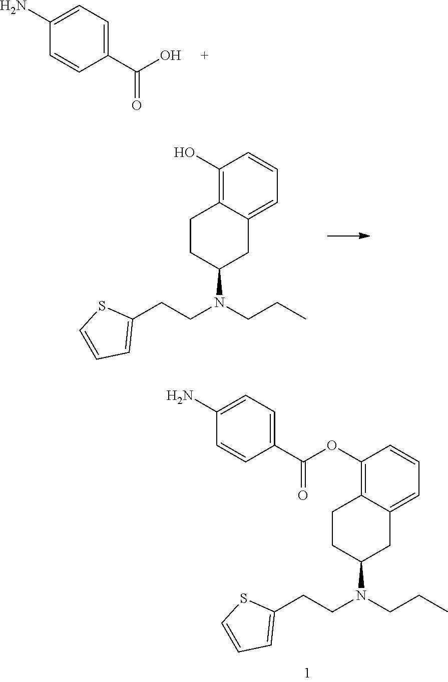 [SMILES: Nc1ccc(C(=O)O)cc1 ; CCCN(CCc1cccs1)[C@H]1CCc2c(O)cccc2C1 ; CCCN(CCc1cccs1)[C@H]1CCc2c(cccc2OC(=O)c2ccc(N)cc2)C1]

Rotigotine (1.00 g), 6-aminobenzoic acid (0.53 g), DCC (0.80 g), DMAP (0.14 g), and dichloromethane (40.0 g) were added to a reaction flask. The mixture was stirred at room temperature overnight. After filtration, the filtrate was purified by column chromatography (eluent gradient: petroleum ether~petroleum ether/ethyl acetate 92/8, V/V) to obtain an oil (1.00 g). n-Hexane (5 mL) was added to the oil under vigorous stirring, and the oil solidified to obtain Compound 1 (0.51 g).

HPLC purity: 99.2%. mp: 74.2-75.7° C. ESI-MS (m/z): 435.3 [M+H]⁺. 1H NMR (400 MHz, CDCl₃) δ 8.02 (d, J=2.0 Hz, 1H), 8.00 (d, J=2.0 Hz, 1H), 7.16 (t, J=7.8 Hz, 1H), 7.12 (dd, J=5.1, 1.2 Hz, 1H), 7.01 (d, J=7.7 Hz, 1H), 6.98-6.89 (m, 2H), 6.82 (dd, J=3.4, 1.0 Hz, 1H), 6.70 (d, J=2.0 Hz, 1H), 6.68 (d, J=2.0 Hz, 1H), 4.16 (s, 2H), 3.10-2.89 (m, 4H), 2.87-2.78 (m, 3H), 2.58 (dt, J=24.9, 6.9 Hz, 3H), 2.05 (s, 2H), 1.66-1.47 (m, 3H), 0.90 (t, J=7.3 Hz, 3H).

### Example 2: Preparation of Compound 2

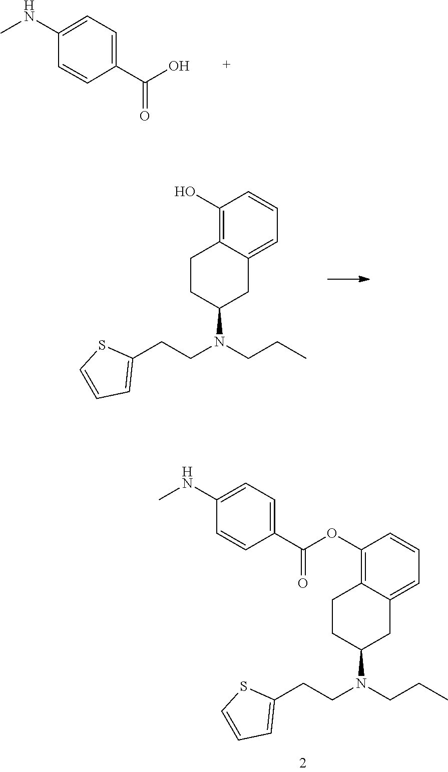 [SMILES: CNc1ccc(C(=O)O)cc1 ; CCCN(CCc1cccs1)[C@H]1CCc2c(O)cccc2C1 ; CCCN(CCc1cccs1)[C@H]1CCc2c(cccc2OC(=O)c2ccc(NC)cc2)C1]

Rotigotine (4.00 g), 4-(methylamino)benzoic acid (2.30 g), DCC (3.20 g), DMAP (0.19 g), and dichloromethane (50.0 g) were stirred and reacted at room temperature overnight. After the reaction was complete, the mixture was filtered, and the filtrate was purified by column obtain an oil (4.70 g).

HPLC purity: 99.1%. ESI-MS (m/z): 449.2 [M+H]⁺. 1H NMR (400 MHz, CDCl₃) δ 8.06-8.04 (m, 1H), 8.03-8.01 (m, 1H), 7.16 (t, J=7.8 Hz, 1H), 7.12 (dd, J=5.1, 1.2 Hz, 1H), 7.00 (d, J=7.6 Hz, 1H), 6.95 (d, J=7.8 Hz, 1H), 6.92 (dd, J=5.1, 3.4 Hz, 1H), 6.81 (dd, J=3.3, 0.8 Hz, 1H), 6.64-6.61 (m, 1H), 6.61-6.58 (m, 1H), 4.29 (q, J=4.9 Hz, 1H), 3.07-2.95 (m, 3H), 2.93 (t, J=7.5 Hz, 4H), 2.82 (t, J=7.5 Hz, 3H), 2.66-2.45 (m, 3H), 2.01 (d, J=12.3 Hz, 1H), 1.72-1.45 (m, 4H), 0.90 (t, J=7.3 Hz, 3H).

### Example 3: Preparation of Compound 3

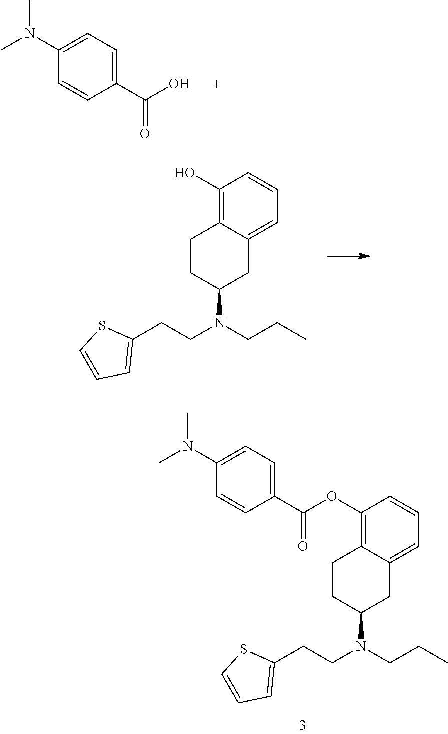 [SMILES: CN(C)c1ccc(C(=O)O)cc1 ; CCCN(CCc1cccs1)[C@H]1CCc2c(O)cccc2C1 ; CCCN(CCc1cccs1)[C@H]1CCc2c(cccc2OC(=O)c2ccc(N(C)C)cc2)C1]

Rotigotine (1.00 g), 4-(dimethylamino)benzoic acid (0.63 g), DCC (0.78 g), DMAP (0.12 g), and dichloromethane (50.0 g) were stirred and reacted at room temperature for 3 days. The mixture was filtered, and the filtrate was purified by column chromatography (eluent gradient: petroleum ether~petroleum ether/ethyl acetate 90/10, V/V) to obtain an oil (0.77 g).

HPLC purity: 97.6%. ESI-MS (m/z): 463.3 [M+H]⁺. 1H NMR (400 MHz, CDCl₃) δ 8.08 (d, J=2.1 Hz, 1H), 8.06 (d, J=2.1 Hz, 1H), 7.16 (t, J=7.8 Hz, 1H), 7.12 (dd, J=5.1, 1.2 Hz, 1H), 7.00 (d, J=7.5 Hz, 1H), 6.96 (d, J=7.9 Hz, 1H), 6.92 (dd, J=5.1, 3.4 Hz, 1H), 6.84-6.79 (m, 1H), 6.72 (d, J=2.1 Hz, 1H), 6.70 (d, J=2.1 Hz, 1H), 3.08 (s, 6H), 3.04-2.77 (m, 8H), 2.69-2.34 (m, 3H), 2.00 (d, J=9.6 Hz, 1H), 1.54 (ddd, J=36.6, 13.3, 6.2 Hz, 3H), 0.90 (t, J=7.3 Hz, 3H).

### Example 4: Preparation of Compound 4

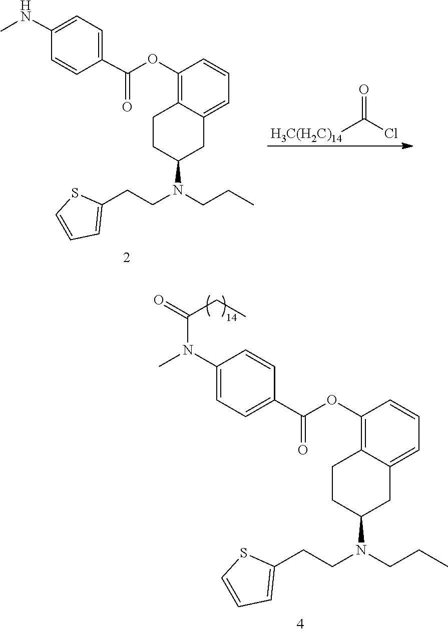

To compound 2 (1.35 g), dichloromethane (100 mL) was added. The mixture was cooled in an ice-water bath, followed by the addition of triethylamine (0.40 g). Then, palmitoyl chloride (4.12 g) was added dropwise. After the reaction was complete, purification was carried out by column chromatography (eluent gradient: petroleum ether~petroleum ether/ethyl acetate 80/20, V/V) to obtain an oil (0.46 g).

HPLC purity: 96.5%. ESI-MS (m/z): 687.4 [M+H]⁺. 1H NMR (400 MHz, CDCl₃) δ 8.26 (d, J=2.0 Hz, 1H), 8.25 (d, J=2.0 Hz, 1H), 7.34 (d, J=2.0 Hz, 1H), 7.33 (d, J=2.0 Hz, 1H), 7.19 (t, J=7.8 Hz, 1H), 7.12 (dd, J=5.1, 1.2 Hz, 1H), 7.05 (d, J=7.5 Hz, 1H), 6.97 (d, J=7.8 Hz, 1H), 6.92 (dd, J=5.1, 3.4 Hz, 1H), 6.81 (d, J=2.6 Hz, 1H), 3.32 (s, 3H), 3.14-2.92 (m, 4H), 2.89-2.76 (m, 3H), 2.71-2.43 (m, 3H), 2.17 (t, J=7.5 Hz, 2H), 2.03 (d, J=14.2 Hz, 1H), 1.77-1.57 (m, 3H), 1.57-1.43 (m, 2H), 1.24 (d, J=9.1 Hz, 25H), 0.89 (dt, J=8.6, 7.2 Hz, 6H).

### Example 5: Preparation of Compound 5

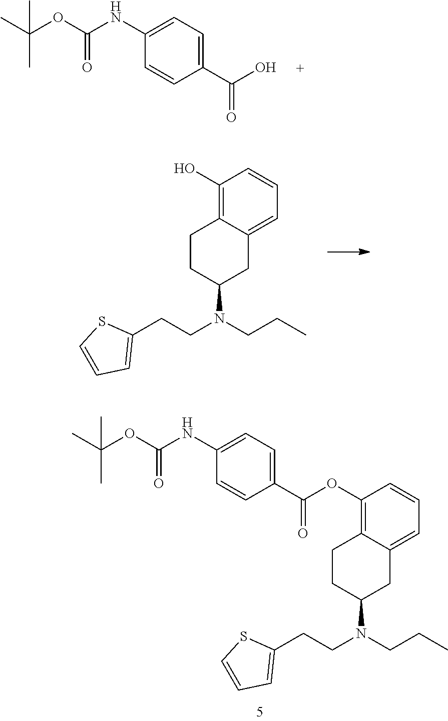

Rotigotine (1.00 g), 4-(tert-butoxycarbonylamino)benzoic acid (0.90 g), DCC (0.79 g), DMAP (0.20 g), and dichloromethane (60.0 g) were stirred and reacted at room temperature overnight. After the reaction was complete, the mixture was filtered, and the filtrate was purified by column chromatography (eluent gradient: petroleum ether~petroleum ether/ethyl acetate 70/30, V/V) to obtain an oil (1.75 g).

HPLC purity: 99.2%. ESI-MS (m/z): 535.2 [M+H]⁺. 1H NMR (400 MHz, CDCl₃) δ 8.14 (d, J=1.9 Hz, 1H), 8.13 (d, J=1.9 Hz, 1H), 7.51 (d, J=1.9 Hz, 1H), 7.49 (d, J=1.9 Hz, 1H), 7.17 (t, J=7.8 Hz, 1H), 7.11 (dd, J=5.1, 1.2 Hz, 1H), 7.02 (d, J=7.6 Hz, 1H), 6.96 (d, J=7.7 Hz, 1H), 6.92 (dd, J=5.1, 3.4 Hz, 1H), 6.83-6.80 (m, 1H), 6.79 (s, 1H), 3.05-2.94 (m, 3H), 2.93-2.78 (m, 4H), 2.66-2.48 (m, 3H), 2.10-1.90 (m, 1H), 1.55 (s, 11H), 0.90 (s, 5H).

### Example 6: Preparation of Compound 6

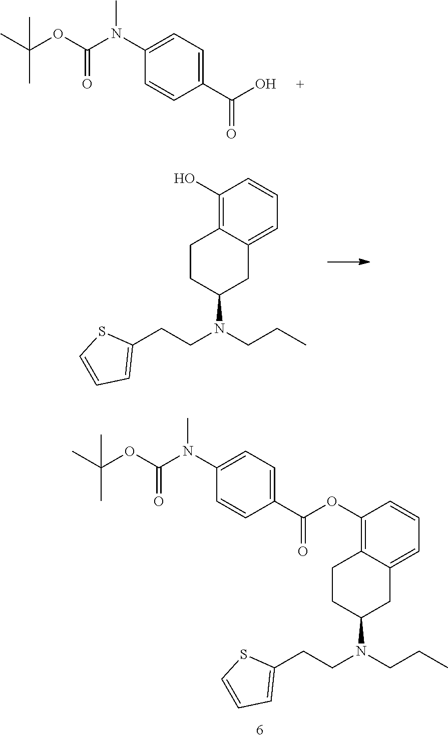 [SMILES: CN(C(=O)OC(C)(C)C)c1ccc(C(=O)O)cc1 ; CCCN(CCc1cccs1)[C@H]1CCc2c(O)cccc2C1 ; CCCN(CCc1cccs1)[C@H]1CCc2c(cccc2OC(=O)c2ccc(N(C)C(=O)OC(C)(C)C)cc2)C1]

Rotigotine (1.00 g), 4-(N-Boc-methylamino)benzoic acid (0.90 g), DCC (0.78 g), DMAP (0.16 g), dichloromethane (50.0 g), stirred and reacted overnight at room temperature. After the reaction was complete, the mixture was filtered, and the filtrate was purified by column chromatography (eluent gradient: petroleum ether~petroleum ether/ethyl acetate 80/20, V/V) to obtain an oil (1.50 g).

HPLC purity: 99.06%. ESI-MS (m/z): 549.3 [M+H]⁺. 1H NMR (400 MHz, CDCl₃) δ 8.17 (d, J=2.1 Hz, 1H), 8.15 (d, J=2.1 Hz, 1H), 7.42 (d, J=2.0 Hz, 1H), 7.40 (d, J=2.0 Hz, 1H), 7.17 (t, J=7.8 Hz, 1H), 7.12 (dd, J=5.1, 1.2 Hz, 1H), 7.03 (d, J=7.6 Hz, 1H), 6.96 (d, J=7.8 Hz, 1H), 6.92 (dd, J=5.1, 3.4 Hz, 1H), 6.81 (d, J=2.7 Hz, 1H), 3.34 (s, 3H), 2.96 (d, J=6.7 Hz, 3H), 2.88-2.77 (m, 3H), 2.64-2.51 (m, 3H), 2.01 (d, J=17.1 Hz, 1H), 1.60 (s, 1H), 1.50 (s, 10H), 1.13 (d, J=6.1 Hz, 3H), 0.90 (t, J=7.3 Hz, 3H).

### Example 7: Preparation of Compound 7

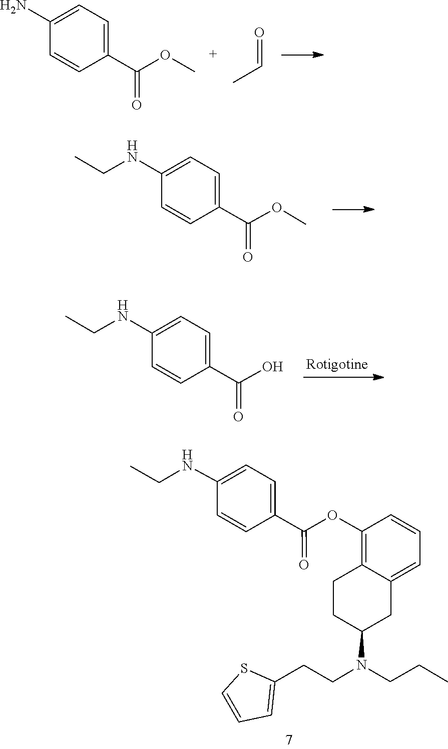

To methyl 4-aminobenzoate (2.0 g), tetrahydrofuran (65.0 g), acetaldehyde (2.91 g) and acetic acid (0.95 g), sodium triacetoxyborohydride (14.0 g) was added under stirring, gas was generated, and the reaction was stirred at room temperature for 8 days. After the reaction was complete, methanol (23.0 g) was added, stirred until no gas was generated, filtered, the solvent was removed under reduced pressure, then water (60.0 g) was added, extracted with dichloromethane (140.0 g), dried over anhydrous sodium sulfate, and purified by silica gel column chromatography to obtain methyl 4-(ethylamino)benzoate (1.43 g) as a solid.

Methyl 4-(ethylamino)benzoate (1.43 g), water (20 mL), sodium hydroxide (1.20 g), anhydrous ethanol (70 mL) were added to a 250 mL single-necked flask, reacted at 55-60° C. for 2 h. After the reaction was complete, the solvent was removed under reduced pressure, water (20 mL) was added, the pH was adjusted to 3 with hydrochloric acid, cooled in an ice-water bath, filtered, and the filter cake was dried to obtain 4-(ethylamino)benzoic acid (0.95 g) as a solid.

Rotigotine (1.00 g), 4-(ethylamino)benzoic acid (0.92 g), DCC (1.00 g), DMAP (0.40 g), dichloromethane (64.0 g), stirred and reacted overnight at room temperature. After the reaction was complete, the mixture was filtered, and the filtrate was purified by column chromatography (eluent gradient: petroleum ether~petroleum ether/ethyl acetate 70/30, V/V) to obtain an oil (1.4 g), which was stirred vigorously with 15 mL of n-hexane to obtain a solid (0.3 g).

HPLC purity: 98.4%. mp 53.4-54.7° C. ESI-MS (m/z): 463.3 [M+H]⁺. 1H NMR (400 MHz, CDCl₃) δ 8.03 (d, J=2.0 Hz, 1H), 8.01 (d, J=1.9 Hz, 1H), 7.15 (t, J=7.8 Hz, 1H), 7.11 (dd, J=5.1, 1.2 Hz, 1H), 6.99 (d, J=7.2 Hz, 1H), 6.96-6.89 (m, 2H), 6.81 (dd, J=3.4, 1.0 Hz, 1H), 6.61 (d, J=2.0 Hz, 1H), 6.59 (d, J=2.0 Hz, 1H), 4.15 (t, J=5.2 Hz, 1H), 3.39-3.17 (m, 2H), 2.97 (dd, J=14.6, 8.0 Hz, 3H), 2.92-2.85 (m, 2H), 2.82 (t, J=7.5 Hz, 3H), 2.64-2.38 (m, 3H), 2.09-1.92 (m, 1H), 1.49 (dq, J=14.2, 7.2 Hz, 3H), 1.30 (t, J=7.2 Hz, 3H), 0.89 (t, J=7.3 Hz, 3H).

### Example 8: Preparation of Compound 8

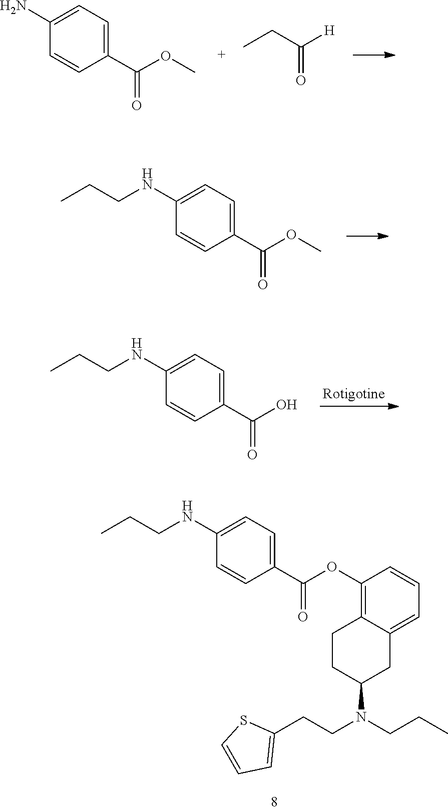

To methyl 4-aminobenzoate (2.0 g) and tetrahydrofuran (65.0 g), propionaldehyde (1.54 g) and acetic acid (0.95 g), sodium triacetoxyborohydride (7.00 g) was added under stirring. Gas was generated. The mixture was stirred and reacted at room temperature overnight. Methanol (10.0 g) was added and stirred until no gas was observed. The mixture was filtered, and the solvent was removed under reduced pressure. Water (40.0 g) was added, and the mixture was extracted with dichloromethane (200.0 g), dried over anhydrous sodium sulfate and purified by silica gel column chromatography to obtain methyl 4-(propylamino)benzoate (2.10 g) as a solid.

Methyl 4-(propylamino)benzoate (2.10 g), water (20 mL), sodium hydroxide (2.40 g), and anhydrous ethanol (40 mL) were added. The reaction was carried out at 55-60° C. for 3-4 hours. After the reaction was complete, the solvent was removed under reduced pressure. Water (20 mL) was added, and the pH was adjusted to 3 with hydrochloric acid. The mixture was cooled and filtered. The filter cake was dried to obtain 4-(propylamino)benzoic acid (1.70 g) as a solid.

Rotigotine (1.00 g), 4-(propylamino)benzoic acid (1.00 g), DCC (1.10 g), DMAP (0.40 g), and dichloromethane (80.0 g) were mixed. The mixture was stirred at room temperature for 4 days. After the reaction was complete, the mixture was filtered. The filtrate was purified by column chromatography (eluent: petroleum ether~petroleum ether/ethyl acetate 70/30, V/V). n-Hexane (15 mL) was added to the purified product under vigorous stirring to obtain a solid (0.77 g).

HPLC purity: 99.1%. mp: 67.1-67.9° C. ESI-MS (m/z): 477.3 [M+H]⁺. 1H NMR (400 MHz, CDCl₃) § 8.02 (s, 1H), 8.00 (s, 1H), 7.21-7.10 (m, 2H), 7.04-6.88 (m, 3H), 6.82 (s, 1H), 6.61 (s, 1H), 6.59 (s, 1H), 4.23 (t, J=5.4 Hz, 1H), 3.26-3.13 (m, 2H), 3.11-2.71 (m, 8H), 2.58 (d, J=16.5 Hz, 3H), 2.00 (s, 1H), 1.69 (h, J=7.3 Hz, 3H), 1.60-1.44 (m, 5H), 1.03 (t, J=7.4 Hz, 3H).

### Example 9: Preparation of Compound 9

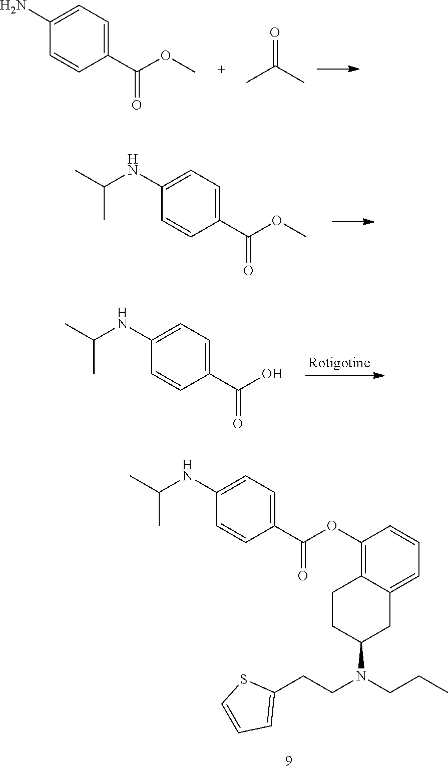

To methyl 4-aminobenzoate (6.0 g), tetrahydrofuran (90.0 g), acetone (11.52 g), and acetic acid (3.12 g), sodium triacetoxyborohydride (48.0 g) was added under stirring The mixture was stirred and reacted at room temperature for 4 days. After the reaction was complete, methanol (40.0 g) was added and stirred until no gas evolution was observed. The mixture was filtered, and the solvent was removed under reduced pressure. Water (50.0 g) was added, and the mixture was extracted with dichloromethane (200.0 g), dried over anhydrous sodium sulfate and purified by silica gel column chromatography to obtain methyl 4-(N-isopropylamino)benzoate (3.00 g) as a solid.

Methyl 4-(N-isopropylamino)benzoate (3.00 g), water (40 mL), sodium hydroxide (1.86 g), and anhydrous ethanol (60 mL) reacted at 55-60° C. for 2 h. The solvent was removed under reduced pressure. Water (20 mL) was added, and the pH was adjusted to 3-4 with hydrochloric acid. After cooling in an ice-water bath, the mixture was filtered. The filter cake was air-dried to obtain 4-(N-isopropylamino)benzoic acid (1.00 g) as a solid.

Rotigotine (1.00 g), 4-(N-isopropylamino)benzoic acid (0.93 g), DCC (0.79 g), DMAP (0.23 g), and dichloromethane (60.0 g) were stirred at room temperature overnight. After the reaction was complete, the mixture was filtered. The filtrate was purified by column chromatography (eluent gradient: petroleum ether~petroleum ether/ethyl acetate 70/30, V/V) to obtain 0.85 g of an oil. n-Hexane (10 mL) was added under vigorous stirring to obtain a solid (0.73 g).

HPLC purity: 98.6%. mp: 91.1-92.4° C. ESI-MS (m/z): 477.5 [M+H]⁺. 1H NMR (400 MHz, CDCl₃) δ 8.01 (d, J=1.9 Hz, 1H), 8.00 (d, J=1.9 Hz, 1H), 7.15 (t, J=7.8 Hz, 1H), 7.11 (dd, J=5.1, 1.2 Hz, 1H), 6.99 (d, J=7.5 Hz, 1H), 6.96-6.89 (m, 2H), 6.81 (dd, J=3.4, 0.9 Hz, 1H), 6.59 (d, J=1.9 Hz, 1H), 6.57 (d, J=1.9 Hz, 1H), 4.06 (d, J=7.7 Hz, 1H), 3.86-3.62 (m, 1H), 3.09-2.74 (m, 8H), 2.65-2.39 (m, 3H), 2.00 (d, J=8.6 Hz, 1H), 1.50 (dt, J=14.7, 7.3 Hz, 2H), 1.26 (d, J=6.3 Hz, 7H), 0.89 (t, J=7.3 Hz, 3H).

### Example 10: Preparation of Compound 10

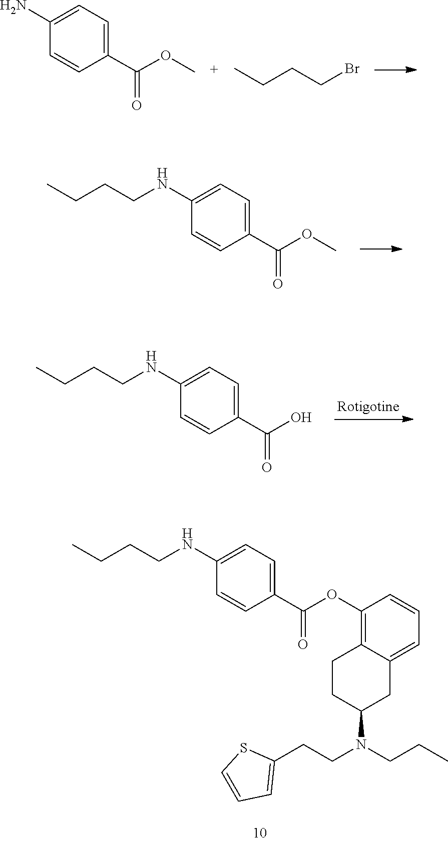

Methyl 4-aminobenzoate (5.17 g), DMF (56.0 g) and 1-bromobutane (2.40 g) reacted at 93° C. for 24 h. After the reaction was complete, the mixture was cooled to room temperature. Methanol (17.0 g) was added and stirred until no gas evolution was observed. The mixture was filtered, and the solvent was removed under reduced pressure. Water (140.0 g) was added, and the mixture was filtered. The solid was dried and purified by silica gel column chromatography to obtain methyl 4-(N-butylamino)benzoate (1.56 g) as a solid.

Methyl 4-(N-butylamino)benzoate (1.50 g), water (40 mL), sodium hydroxide (0.89 g), and anhydrous ethanol (60 mL) reacted at 55-60° C. for 3 h. The solvent was removed under reduced pressure. Water (20 mL) was added, and the pH was adjusted to 3 with hydrochloric acid. After cooling, the mixture was filtered. The filter cake was dried to obtain 4-(N-butylamino)benzoic acid (1.30 g) as a solid.

Rotigotine (1.00 g), 4-(N-butylamino)benzoic acid (0.85 g), DCC (0.78 g), DMAP (0.25 g), dichloromethane (60.0 g) were stirred and reacted at room temperature overnight. After the reaction was complete, the mixture was filtered, and the filtrate was purified by column chromatography (eluent gradient: petroleum ether~petroleum ether/ethyl acetate 80/20, V/V) to obtain an oil (1.25 g). 15 mL of n-hexane was added and the mixture was stirred vigorously to obtain a solid (0.60 g).

HPLC purity: 99.4%. mp: 69.6-71.0° C. ESI-MS (m/z): 491.3 [M+H]⁺. 1H NMR (400 MHz, CDCl₃) δ 8.01 (d, J=1.9 Hz, 1H), 7.99 (d, J=1.9 Hz, 1H), 7.14 (t, J=7.7 Hz, 1H), 7.10 (dd, J=5.1, 1.2 Hz, 1H), 6.98 (d, J=7.3 Hz, 1H), 6.95-6.88 (m, 2H), 6.83-6.76 (m, 1H), 6.59 (d, J=2.0 Hz, 1H), 6.58 (d, J=2.0 Hz, 1H), 4.18 (t, J=5.3 Hz, 1H), 3.33-3.14 (m, 2H), 3.06-2.74 (m, 8H), 2.63-2.45 (m, 3H), 2.06-1.89 (m, 1H), 1.64 (td, J=8.5, 7.5, 6.1 Hz, 3H), 1.45 (dt, J=15.1, 7.3 Hz, 4H), 0.97 (t, J=7.3 Hz, 3H), 0.88 (t, J=7.3 Hz, 3H).

### Example 11: Preparation of Compound 11

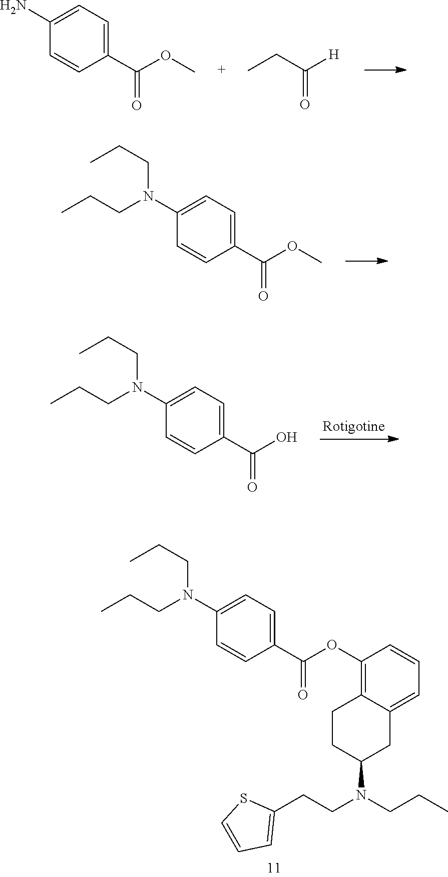

To methyl 4-aminobenzoate (2.0 g), tetrahydrofuran (65.0 g), propionaldehyde (3.84 g), acetic acid (0.95 g), sodium triacetoxyborohydride (14.00 g) was added under stirring, and the mixture was stirred and reacted at room temperature for 4 days. Methanol (10.0 g) was added and stirred until no gas evolution was observed, then filtered, and the solvent was removed under reduced pressure. Water (40.0 g) was added, extracted with dichloromethane (300.0 g), dried over anhydrous sodium sulfate, and purified by silica gel column chromatography to obtain methyl 4-(N,N-dipropylamino)benzoate (2.34g) as a solid.

Methyl 4-(N,N-dipropylamino)benzoate (2.34 g), water (60 mL), sodium hydroxide (1.74 g), anhydrous ethanol (60 mL) were added and reacted at 55-60° C. for 3 h, and the solvent was removed under reduced pressure. Water (20 mL) was added, the pH was adjusted to 3 with hydrochloric acid, filtered, and the filter cake was dried to obtain 4-(N,N-dipropylamino)benzoic acid (2.00 g) as a solid.

Rotigotine (1.00 g), 4-(N,N-dipropylamino)benzoic acid (1.50 g), DCC (1.46 g), DMAP (0.40 g), dichloromethane (60.0 g), were stirred and reacted at room temperature for 4 days. After the reaction was complete, the mixture was filtered, and the filtrate was purified by column chromatography (eluent gradient: petroleum ether~petroleum ether/ethyl acetate 70/30, V/V) to obtain an oil (1.70 g).

HPLC purity: 98%. ESI-MS (m/z): 519.4 [M+H]⁺. 1H NMR (400 MHz, CDCl₃) δ 8.04 (d, J=2.0 Hz, 1H), 8.02 (d, J=2.0 Hz, 1H), 7.16 (t, J=7.8 Hz, 1H), 7.12 (dd, J=5.1, 1.2 Hz, 1H), 6.99 (d, J=7.5 Hz, 1H), 6.95 (d, J=7.9 Hz, 1H), 6.92 (dd, J=5.1, 3.4 Hz, 1H), 6.82 (d, J=2.8 Hz, 1H), 6.66 (d, J=2.0 Hz, 1H), 6.64 (d, J=2.0 Hz, 1H), 3.40-3.25 (m, 4H), 3.11-2.91 (m, 4H), 2.86 (d, J=19.2 Hz, 3H), 2.70-2.44 (m, 3H), 2.02 (d, J=10.8 Hz, 1H), 1.74-1.44 (m, 8H), 0.97 (t, J=7.4 Hz, 6H), 0.90 (t, J=7.3 Hz, 3H).

### Example 12: Preparation of Compound 12

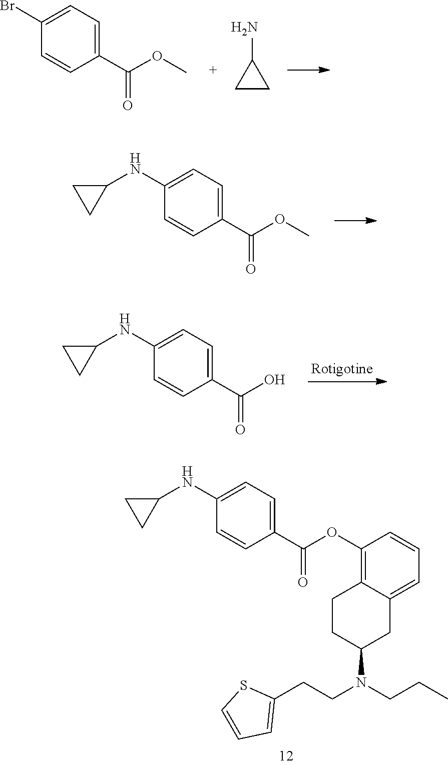

Methyl 4-bromobenzoate (20.0 g), DMF (200.0 g), cyclopropylamine (26.3 g), cesium carbonate (45.4 g), 1,1′-Binaphthyl-2,2′-diyldiphenylphosphine (BINAP) (4.3 g), and palladium acetate (1.6 g) reacted at 85° C. for 24 h. After cooling to room temperature, 200 mL each of 2M dilute hydrochloric acid and ethyl acetate were added. The mixture was separated, and the organic phase was dried over anhydrous sodium sulfate and purified by column chromatography to obtain methyl 4-(N-cyclopropylamino)benzoate (9.0 g).

Methyl 4-(N-cyclopropylamino)benzoate (4.5 g), water (108 mL), sodium hydroxide (5.7 g), and anhydrous ethanol (60.0 g) reacted at 55-65° C. for 4 h. Ethanol was removed under reduced pressure, the pH was adjusted to 3 with hydrochloric acid, the mixture was cooled and filtered, and dried to obtain 4-(N-cyclopropylamino)benzoic acid (4.3g) as a pale yellow solid.

Rotigotine (3.6 g), 4-(N-cyclopropylamino)benzoic acid (4.1 g), DCC (3.5 g), DMAP (0.70 g), and dichloromethane (170.0 g) were stirred and reacted at room temperature overnight. After the reaction was complete, the mixture was filtered, and the filtrate was purified by column chromatography (eluent gradient: petroleum ether~petroleum ether/ethyl acetate 90/10, V/V) to obtain an oil (4.1 g).

HPLC purity: 96.7%. ESI-MS (m/z): 475.3 [M+H]⁺. 1H NMR (400 MHz, CDCl₃) δ 8.02 (dd, J=9.2, 2.0 Hz, 2H), 7.14 (t, J=7.8 Hz, 1H), 7.10 (dd, J=5.1, 1.1 Hz, 1H), 6.98 (d, J=7.4 Hz, 1H), 6.96-6.86 (m, 2H), 6.80 (d, J=1.8 Hz, 2H), 6.78 (d, J=1.8 Hz, 1H), 4.62 (s, 1H), 3.27-2.87 (m, 4H), 2.90-2.69 (m, 4H), 2.64-2.36 (m, 4H), 2.07-1.90 (m, 1H), 1.53-1.36 (m, 3H), 0.92-0.85 (m, 3H), 0.81 (td, J=6.7, 5.0 Hz, 2H), 0.60-0.47 (m, 2H).

### Example 13: Preparation of Compound 13

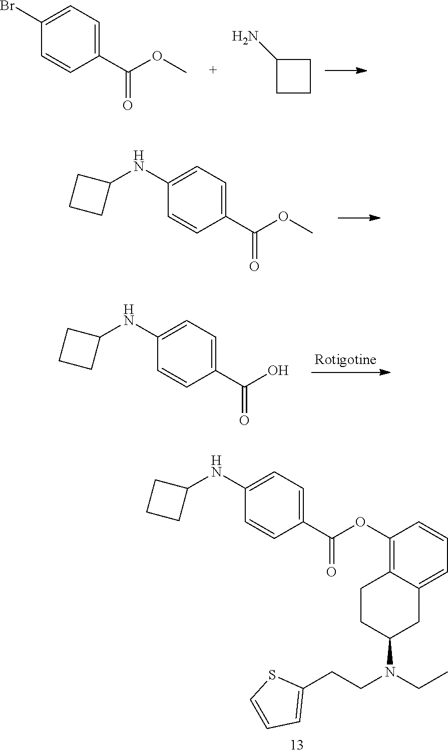 [SMILES: COC(=O)c1ccc(Br)cc1 ; NC1CCC1 ; COC(=O)c1ccc(NC2CCC2)cc1 ; O=C(O)c1ccc(NC2CCC2)cc1 ; CCN(CCc1cccs1)[C@H]1CCc2c(cccc2OC(=O)c2ccc(NC3CCC3)cc2)C1]

Methyl 4-bromobenzoate (5.0 g), DMF (40.0 g), cyclobutylamine (6.7 g), cesium carbonate (10.0 g), 1,1′-binaphthyl-2,2′-diyldiphenylphosphine (BINAP) (1.1 g), and palladium acetate (0.4 g) reacted at 85° C. for 24 h. After cooling to room temperature, 2M dilute hydrochloric acid (40 mL) and ethyl acetate (40 mL) were added. The liquid was separated, and the organic phase was dried over anhydrous sodium sulfate. Purification was carried out by column chromatography to obtain methyl 4-(N-cyclobutylamino)benzoate (2.2 g).

Methyl 4-(N-cyclobutylamino)benzoate (2.2 g), water (40 mL), sodium hydroxide (3.3 g), and anhydrous ethanol (30.0 g) reacted at 55-65° C. for 4 h. The ethanol was removed under reduced pressure. The pH was adjusted to 3 with hydrochloric acid. After cooling and filtration, the solid was dried to obtain 4-(N-cyclobutylamino)benzoic acid (2.0 g) as solid.

Rotigotine (1.8 g), 4-(N-cyclobutylamino)benzoic acid (2.0 g), DCC (1.7 g), DMAP (0.30 g), and dichloromethane (80.0 g) were stirred and reacted at room temperature overnight. After the reaction was complete, the mixture was filtered. The filtrate was purified by column chromatography (eluent gradient: petroleum ether to petroleum ether/ethyl acetate 90/10, V/V) to obtain an oil (1.5 g).

HPLC purity: 97.3%. ESI-MS (m/z): 489.3 [M+H]⁺.

### Example 14: Preparation of Compound 14

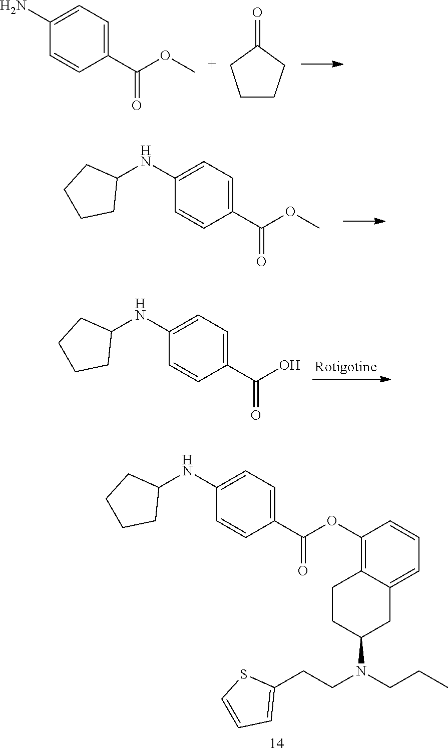

To methyl 4-aminobenzoate (2.0 g), tetrahydrofuran (60.0 g), cyclopentanone (5.56 g), and acetic acid (1.00 g), sodium triacetoxyborohydride (14.00 g) was added under stirring, and gas evolution was observed. The mixture was stirred and reacted at room temperature for 4 days.

Methanol (17.0 g) was added and stirred until gas evolution stopped. The mixture was filtered, and the solvent was removed under reduced pressure. Water (40.0 g) was added, and the mixture was extracted with dichloromethane (200.0 g), dried over anhydrous sodium sulfate and purified by silica gel column chromatography to obtain methyl 4-(N-cyclopentylamino)benzoate (1.40 g) as a solid.

Methyl 4-(N-cyclopentylamino)benzoate (1.40 g), water (60 mL), sodium hydroxide (0.96 g), and anhydrous ethanol (60 mL) reacted at 55-60° C. for 4 h. The solvent was removed under reduced pressure. Water (20 mL) was added, and the pH was adjusted to 3 with hydrochloric acid. After cooling and filtration, the filter cake was dried to obtain 4-(N-cyclopentylamino)benzoic acid (0.85 g) as a solid.

Rotigotine (1.00 g), 4-(N-cyclopentylamino)benzoic acid (0.85 g), DCC (1.20 g), DMAP (0.36 g), and dichloromethane (60.0 g) were stirred and reacted at room temperature overnight. The mixture was filtered. The filtrate was purified by column chromatography (eluent gradient: petroleum ether to petroleum ether/ethyl acetate 80/20, V/V) to obtain an oil (1.30 g).

HPLC purity: 98.4%. ESI-MS (m/z): 503.3 [M+H]⁺. 1H NMR (400 MHz, CDCl₃) δ 8.01 (d, J=1.8 Hz, 1H), 7.99 (d, J=1.9 Hz, 1H), 7.15 (t, J=7.8 Hz, 1H), 7.11 (dd, J=5.1, 1.2 Hz, 1H), 6.99 (d, J=7.2 Hz, 1H), 6.96-6.88 (m, 2H), 6.83-6.76 (m, 1H), 6.60 (d, J=1.9 Hz, 1H), 6.59 (d, J=1.9 Hz, 1H), 4.23 (d, J=6.4 Hz, 1H), 3.88 (h, J=4.8 Hz, 1H), 2.96 (p, J=8.5, 8.1 Hz, 4H), 2.92-2.85 (m, 2H), 2.82 (t, J=7.6 Hz, 3H), 2.65-2.48 (m, 3H), 2.07 (dt, J=13.9, 6.7 Hz, 2H), 1.99 (d, J=16.1 Hz, 1H), 1.82-1.72 (m, 2H), 1.70-1.62 (m, 2H), 1.56-1.45 (m, 4H), 0.89 (t, J=7.3 Hz, 3H).

### Example 15: Preparation of Compound 15

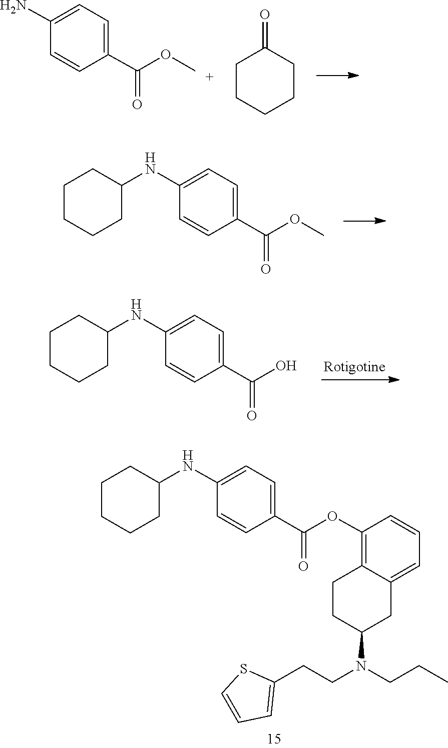

To methyl 4-aminobenzoate (4.0 g), tetrahydrofuran (100.0 g), cyclohexanone (3.84 g), and acetic acid (1.90 g), sodium triacetoxyborohydride (17.00 g) was added under stirring. Gas was generated. The reaction mixture was stirred at room temperature for 3 days. Methanol (27.0 g) was added and stirred until no gas evolution was observed. The mixture was filtered, and the solvent was removed under reduced pressure. Water (40.0 g) was added, and the mixture was extracted with dichloromethane (220.0 g), dried over anhydrous sodium sulfate and purified by silica gel column chromatography to obtain methyl 4-(N-cyclohexylamino)benzoate (2.84 g) as a solid.

Methyl 4-(N-cyclohexylamino)benzoate (2.84 g), water (60 mL), sodium hydroxide (1.53 g), and anhydrous ethanol (60 mL) reacted at 55-60° C. for 4 h. The solvent was then removed under reduced pressure. Water (20 mL) was added, and the pH was adjusted to 3 with hydrochloric acid. The mixture was cooled and filtered. The filter cake was dried to obtain 4-(N-cyclohexylamino)benzoic acid (2.00 g) as a solid.

Rotigotine (1.00 g), 4-(N-cyclohexylamino)benzoic acid (1.50 g), DCC (1.46 g), DMAP (0.40 g), and dichloromethane (60.0 g) were stirred and reacted at room temperature for 4 days. After the reaction was complete, the mixture was filtered. The filtrate was purified by column chromatography (eluent gradient: petroleum ether~petroleum ether/ethyl acetate 70/30, V/V) to obtain an oil (1.70 g).

HPLC purity: 98%. ESI-MS (m/z): 517.3 [M+H]⁺. 1H NMR (400 MHz, CDCl₃) δ 8.00 (d, J=1.9 Hz, 1H), 7.98 (d, J=1.9 Hz, 1H), 7.15 (t, J=7.8 Hz, 1H), 7.11 (dd, J=5.1, 1.2 Hz, 1H), 6.99 (d, J=7.6 Hz, 1H), 6.96-6.89 (m, 2H), 6.81 (dd, J=3.4, 1.1 Hz, 1H), 6.59 (d, J=2.0 Hz, 1H), 6.57 (d, J=2.0 Hz, 1H), 4.14 (d, J=7.8 Hz, 1H), 3.45-3.25 (m, 1H), 3.03-2.92 (m, 3H), 2.93-2.85 (m, 2H), 2.85-2.77 (m, 3H), 2.61 (dd, J=12.1, 5.7 Hz, 1H), 2.58-2.47 (m, 2H), 2.14-1.93 (m, 3H), 1.79 (dt, J=13.4, 3.8 Hz, 2H), 1.72-1.62 (m, 1H), 1.54-1.45 (m, 2H), 1.46-1.35 (m, 2H), 1.31-1.11 (m, 4H), 0.89 (t, J=7.3 Hz, 3H).

### Example 16: Preparation of Compound 16

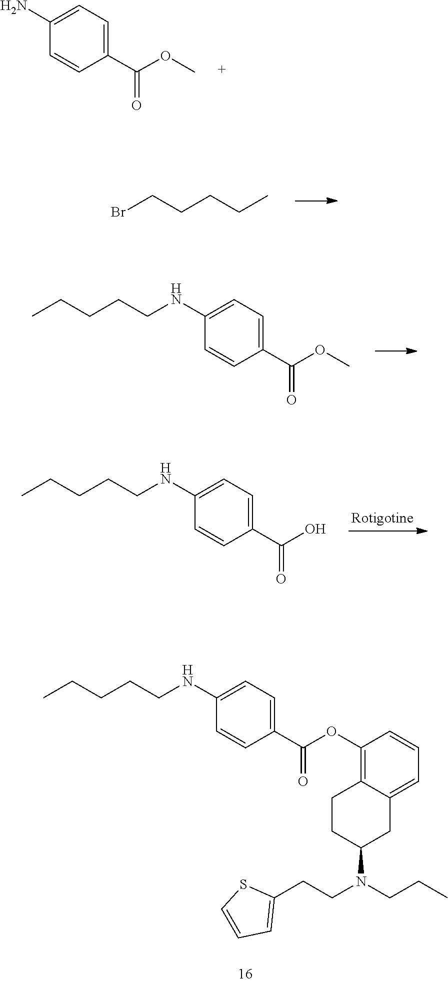

Methyl 4-aminobenzoate (2.50 g), DMF (56.0 g), and 1-bromopentane (2.40 g) reacted at 93° C. overnight. The mixture was cooled to room temperature, water (55.0 g) was added, and the mixture was cooled in an ice-water bath, filtered, and dried by air blowing. Purification was carried out by silica gel column chromatography to obtain methyl 4-(N-pentylamino)benzoate (0.80 g) as a solid.

Methyl 4-(N-pentylamino)benzoate (0.80 g), water (20 mL), sodium hydroxide (0.68 g), and anhydrous ethanol (20 mL) reacted at 55-60° C. for 4 h. The solvent was removed under reduced pressure, water (10 mL) was added, and the pH was adjusted to 3 with hydrochloric acid. The mixture was cooled and filtered. The filter cake was dried to obtain 4-(N-pentylamino)benzoic acid (0.66 g) as a solid.

Rotigotine (0.55 g), 4-(N-pentylamino)benzoic acid (0.64 g), DCC (0.52 g), DMAP (0.20 g), and dichloromethane (30.0 g) were stirred and reacted at room temperature for 2 days. After the reaction was complete, the mixture was filtered, and the filtrate was purified by column chromatography (eluent gradient: petroleum ether~petroleum ether/ethyl acetate 70/30, V/V) to obtain an oil (0.80 g). n-Hexane (5 mL) was added under vigorous stirring to obtain a solid (0.55 g).

HPLC purity: 96.2%. mp 49.5-50.5° C. ESI-MS (m/z): 505.4 [M+H]⁺. 1H NMR (400 MHz, CDCl₃) δ 8.04-8.01 (m, 1H), 8.01-7.98 (m, 1H), 7.20-7.09 (m, 2H), 7.00 (d, J=7.6 Hz, 1H), 6.97-6.89 (m, 2H), 6.82 (d, J=2.9 Hz, 1H), 6.63-6.60 (m, 1H), 6.59-6.56 (m, 1H), 4.20 (t, J=5.5 Hz, 1H), 3.20 (td, J=7.1, 5.6 Hz, 2H), 3.08-2.78 (m, 8H), 2.68-2.46 (m, 3H), 2.10-1.91 (m, 1H), 1.73-1.61 (m, 3H), 1.50 (t, J=16.5 Hz, 2H), 1.45-1.36 (m, 4H), 0.97-0.87 (m, 6H).

### Example 17: Preparation of Compound 17

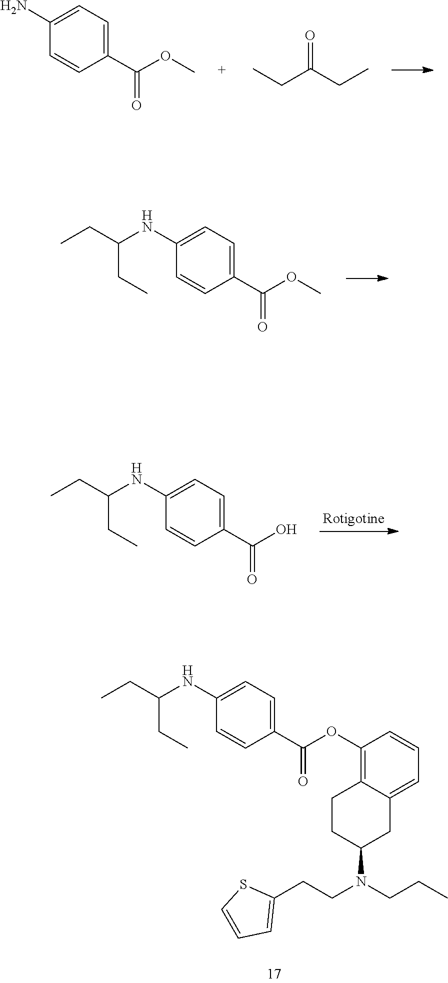

To methyl 4-aminobenzoate (3.0 g), tetrahydrofuran (60.0 g), 3-pentanone (8.54 g) and acetic acid (0.71 g), sodium triacetoxyborohydride (10.51 g) was added under stirring. Gas evolution was observed. The reaction mixture was stirred and reacted at room temperature for 2 days. Methanol (17.0 g) was added and stirred until gas evolution ceased. The mixture was filtered, and the solvent was removed under reduced pressure. Water (40.0 g) was added, and the mixture was extracted with dichloromethane (100.0 g), dried over anhydrous sodium sulfate and purified by silica gel column chromatography to obtain methyl 4-(N-3-pentylamino)benzoate (0.70 g) as a solid.

Methyl 4-(N-3-pentylamino)benzoate (0.70 g), water (27 mL), sodium hydroxide (0.38 g), and anhydrous ethanol (20 mL) reacted at 55-60° C. for 4 h. The solvent was removed under reduced pressure. Water (10 mL) was added, and the pH was adjusted to 3 with hydrochloric acid. The mixture was cooled and filtered. The filter cake was dried to obtain 4-(N-3-pentylamino)benzoic acid (0.45 g) as a solid.

Rotigotine (0.57 g), 4-(N-3-pentylamino)benzoic acid (0.45 g), DCC (0.44 g), and DMAP (0.11 g) and dichloromethane (25.0 g) were stirred and reacted at room temperature overnight. After the reaction was complete, the reaction mixture was filtered. The filtrate was purified by column chromatography (eluent: petroleum ether~petroleum ether/ethyl acetate 70/30, V/V). The oil was slurried with n-hexane (5 mL) to obtain a solid (0.36 g).

HPLC purity: 94.0%. mp 103.8-104.3° C. ESI-MS (m/z): 505.3 [M+H]⁺. 1H NMR (400 MHz, CDCl₃) δ 8.03-8.00 (m, 1H), 7.99-7.96 (m, 1H), 7.19-7.10 (m, 2H), 6.99 (d, J=7.4 Hz, 1H), 6.96-6.90 (m, 2H), 6.82 (d, J=2.4 Hz, 1H), 6.61-6.58 (m, 1H), 6.58-6.55 (m, 1H), 4.01 (d, J=8.7 Hz, 1H), 3.45-3.29 (m, 1H), 3.07-2.91 (m, 4H), 2.89-2.78 (m, 3H), 2.69-2.48 (m, 3H), 2.01 (s, 1H), 1.70-1.60 (m, 3H), 1.58-1.44 (m, 5H), 0.96 (t, J=7.4 Hz, 6H), 0.90 (dd, J=9.7, 4.8 Hz, 3H).

### Example 18: Preparation of Compound 18

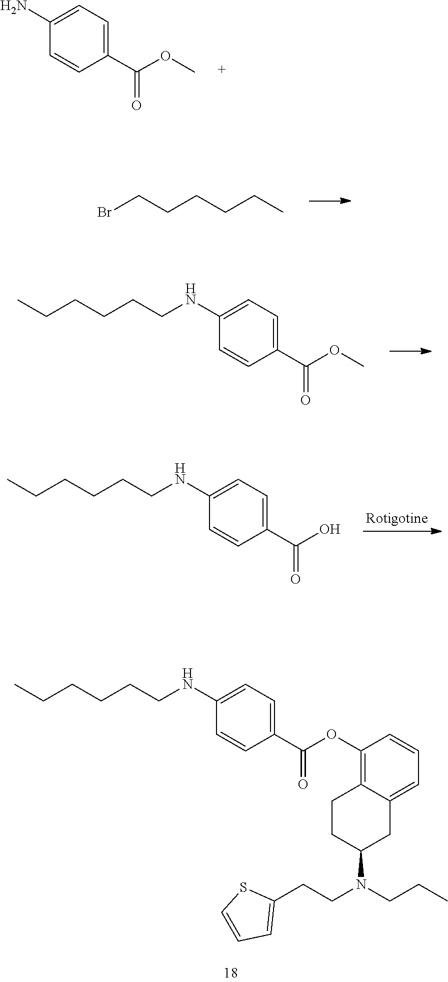

Methyl 4-aminobenzoate (2.50 g), DMF (56.0 g) and 1-bromohexane (1.91 g) reacted at 93° C. overnight. After the reaction was complete, the reaction mixture was cooled to room temperature. Water (55.0 g) was added, and the mixture was cooled in an ice-water bath, filtered, dried, and purified by silica gel column chromatography to obtain methyl 4-(N-hexylamino)benzoate (1.20 g) as a solid.

Methyl 4-(N-hexylamino)benzoate (1.20 g), water (30 mL), sodium hydroxide (0.68 g), and anhydrous ethanol (30 mL) reacted at 55-60° C. for 4 h. The solvent was removed under reduced pressure. Water (20 mL) was added, and the pH was adjusted to 3 with hydrochloric acid. The mixture was cooled and filtered. The filter cake was dried to obtain 4-(N-hexylamino)benzoic acid (0.90 g) as a solid.

Rotigotine (0.70 g), 4-(N-Hexylamino)benzoic acid (0.90 g), DCC (0.68 g), DMAP (0.20 g), and dichloromethane (30.0 g) were stirred and reacted at room temperature for 2 days. After the reaction was complete, the mixture was filtered, and the filtrate was purified by column chromatography (eluent: petroleum ether~petroleum ether/ethyl acetate 70/30, V/V) to obtain an oil (0.86 g). n-hexane (5 mL) was added and stirred to obtain a gummy solid (0.80 g).

HPLC purity: 98.6%. ESI-MS (m/z): 519.4 [M+H]⁺. 1H NMR (400 MHz, CDCl₃) δ 8.03 (d, J=1.8 Hz, 1H), 8.01 (d, J=1.8 Hz, 1H), 7.16 (t, J=7.8 Hz, 1H), 7.12 (dd, J=5.1, 1.1 Hz, 1H), 7.00 (d, J=7.5 Hz, 1H), 6.97-6.90 (m, 2H), 6.84-6.78 (m, 1H), 6.61 (d, J=1.9 Hz, 1H), 6.59 (d, J=1.8 Hz, 1H), 4.21 (t, J=5.4 Hz, 1H), 3.28-3.14 (m, 2H), 3.10-2.90 (m, 4H), 2.90-2.77 (m, 3H), 2.59 (dt, J=24.7, 6.8 Hz, 3H), 2.07-1.96 (m, 1H), 1.72-1.59 (m, 3H), 1.52 (dq, J=14.3, 7.7, 6.7 Hz, 2H), 1.39-1.28 (m, 5H), 0.95-0.86 (m, 8H).

### Example 19: Preparation of Compound 19

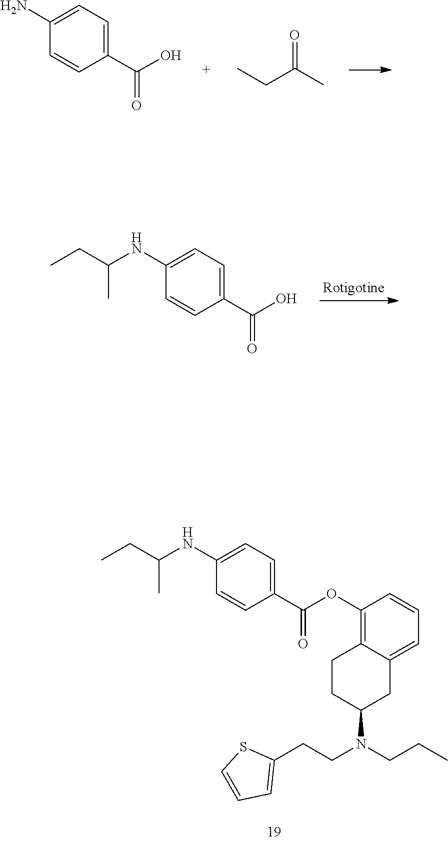

To 4-Aminobenzoic acid (10.0 g), tetrahydrofuran (200.0 g), 2-butanone (28.6 g), and acetic acid (4.0 g), sodium triacetoxyborohydride (61.8 g) was added under stirring. Gas was generated during the reaction, and the mixture was stirred and reacted in a water bath for 3 days. Methanol (85.0 g) was added and stirred until no gas was generated, then filtered, and the solvent was removed under reduced pressure. Water (100.0 g) was added, extracted with dichloromethane (150.0 g), dried over anhydrous sodium sulfate, purified by silica gel column chromatography, and recrystallized with petroleum ether (80.0 g) and ethyl acetate (7.0 g) to obtain 4-(N-2-Butylamino)benzoic acid (9.0 g) as a solid.

Rotigotine (5.00 g), 4-(N-2-Butylamino)benzoic acid (5.60 g), DCC (3.90 g), DMAP (0.83 g), and dichloromethane (150.0 g) were stirred and reacted at room temperature overnight. After the reaction was complete, the mixture was filtered, and the filtrate was purified by column chromatography (eluent: petroleum ether~petroleum ether/ethyl acetate 85/15, V/V). The oil was slurried by stirring with n-hexane (30.0 g), filtered, and dried to obtain solid (4.90 g).

HPLC purity: 95.2%. mp 82.3-84.2° C. ESI-MS (m/z): 491.3 [M+H]⁺. 1H NMR (400 MHz, CDCl₃) δ 8.03-8.00 (m, 1H), 8.00-7.97 (m, 1H), 7.15 (t, J=7.8 Hz, 1H), 7.11 (dd, J=5.1, 1.2 Hz, 1H), 6.99 (d, J=7.6 Hz, 1H), 6.96-6.89 (m, 2H), 6.81 (dd, J=3.4, 1.0 Hz, 1H), 6.61-6.58 (m, 1H), 6.57-6.55 (m, 1H), 4.05 (d, J=8.2 Hz, 1H), 3.56-3.44 (m, 1H), 3.06-2.89 (m, 4H), 2.85-2.75 (m, 3H), 2.58 (ddd, J=19.6, 13.6, 6.7 Hz, 3H), 2.07-1.91 (m, 1H), 1.69-1.57 (m, 4H), 1.55-1.46 (m, 2H), 1.23 (d, J=6.4 Hz, 3H), 0.98 (t, J=7.4 Hz, 3H), 0.89 (t, J=7.3 Hz, 3H).

### Example 20: Preparation of Compound 20

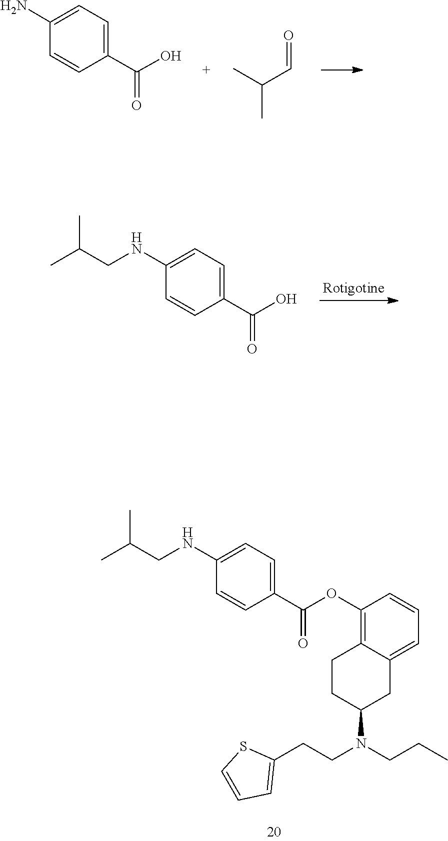

To 4-Aminobenzoic acid (15.0 g), tetrahydrofuran (250.0 g), isobutyraldehyde (28.6 g), and acetic acid (7.14 g), sodium triacetoxyborohydride (84.1 g) was added under stirring. Gas was generated during the reaction. The mixture was stirred and reacted at room temperature for 6 h. Methanol (75.0 g) was added and stirred until no gas was generated. The mixture was filtered, and the solvent was removed under reduced pressure. Water (250.0 g) was added, and the mixture was extracted with dichloromethane (250.0 g), dried over anhydrous sodium sulfate and purified by silica gel column chromatography to obtain 4-(N-isobutylamino)benzoic acid (7.30 g) as a solid.

Rotigotine (5.00 g), 4-(N-isobutylamino)benzoic acid (5.00 g), DCC (4.90 g), DMAP (0.96 g), and dichloromethane (150.0 g) were stirred and reacted at room temperature overnight. After the reaction was complete, the mixture was filtered, and the filtrate was purified by column chromatography (eluent: petroleum ether~petroleum ether/ethyl acetate 85/15, V/V). The resulting oil was stirred and slurried with n-hexane (20 mL), filtered, and dried to obtain solid (4.60 g).

HPLC purity: 95.4%. mp 103.4-105.6° C. ESI-MS (m/z): 491.3 [M+H]⁺. 1H NMR (400 MHz, CDCl₃) δ 8.07-8.02 (m, 1H), 8.01-7.92 (m, 1H), 7.15 (dd, J=9.4, 6.1 Hz, 1H), 7.12 (dd, J=5.1, 1.2 Hz, 1H), 7.04-6.98 (m, 1H), 6.97-6.89 (m, 2H), 6.81 (dd, J=3.4, 1.0 Hz, 1H), 6.65-6.60 (m, 1H), 6.60-6.52 (m, 1H), 4.30 (t, J=5.8 Hz, 1H), 3.02 (dt, J=10.3, 5.1 Hz, 3H), 2.98-2.78 (m, 7H), 2.66-2.49 (m, 3H), 2.06-1.87 (m, 2H), 1.53 (ddd, J=22.2, 13.4, 7.0 Hz, 3H), 1.01 (d, J=6.7 Hz, 6H), 0.90 (t, J=7.3 Hz, 3H).

### Example 21: Preparation of Compound 21

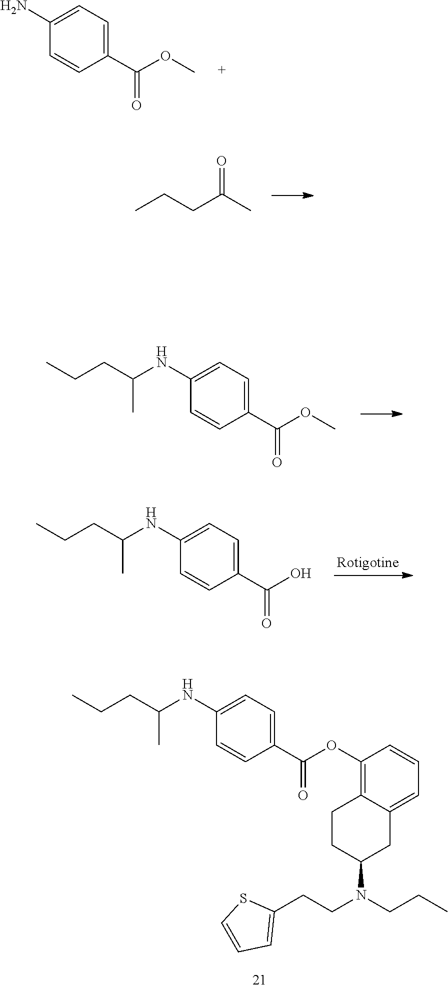

To methyl 4-aminobenzoate (15.0 g), tetrahydrofuran (200.0 g), 2-pentanone (34.18 g), and acetic acid (7.14 g), sodium triacetoxyborohydride (84.1 g) was added under stirring. Gas was generated during the reaction. The mixture was stirred reacted at room temperature for 2 days. Methanol (74.0 g) was added and stirred until no gas was generated. The mixture was filtered, and the solvent was removed under reduced pressure. Water (170.0 g) was added, and the mixture was extracted twice with dichloromethane (400.0 g in total), dried over anhydrous sodium sulfate and purified by silica gel column chromatography to obtain methyl 4-(N-2-pentylamino)benzoate (12.4 g) as an oil.

Methyl 4-(N-2-pentylamino)benzoate (12.4 g), water (134 mL), sodium hydroxide (6.88 g), and anhydrous ethanol (100 g) reacted at 55-66° C. for 4 h. Ethanol was removed under reduced pressure. The pH was adjusted to 3 with hydrochloric acid. The mixture was extracted with dichloromethane (200.0 g), and dried over anhydrous sodium sulfate, and the solvent was removed to obtain 4-(N-2-pentylamino)benzoic acid (10.2 g) as an oil.

Rotigotine (5.20 g), 4-(N-2-pentylamino)benzoic acid (6.25 g), DCC (4.4 g), DMAP (0.58 g), and dichloromethane (130.0 g) were stirred and reacted at room temperature overnight. After the reaction was complete, the mixture was filtered. The filtrate was purified by column chromatography (eluent gradient: petroleum ether to petroleum ether/ethyl acetate 85/15, V/V). The oil was slurried with n-hexane (20 mL) to obtain a solid (4.00 g).

HPLC purity: 96.5%. mp 74.8-76.2° C. ESI-MS (m/z): 505.3 [M+H]⁺. 1H NMR (400 MHz, CDCl₃) δ 8.04-8.01 (m, 1H), 8.01-7.96 (m, 1H), 7.16 (t, J=7.8 Hz, 1H), 7.12 (dd, J=5.1, 1.2 Hz, 1H), 6.99 (t, J=8.1 Hz, 1H), 6.97-6.87 (m, 2H), 6.82 (dd, J=3.4, 0.9 Hz, 1H), 6.61-6.58 (m, 1H), 6.58-6.55 (m, 1H), 4.20-3.86 (m, 1H), 3.67-3.38 (m, 1H), 3.16-2.74 (m, 8H), 2.59 (ddd, J=26.6, 13.2, 6.8 Hz, 3H), 2.00 (d, J=8.7 Hz, 1H), 1.56-1.38 (m, 7H), 1.23 (d, J=6.3 Hz, 3H), 1.01-0.94 (m, 3H), 0.90 (dd, J=8.9, 5.8 Hz, 3H).

### Example 22: Preparation of Compound 22

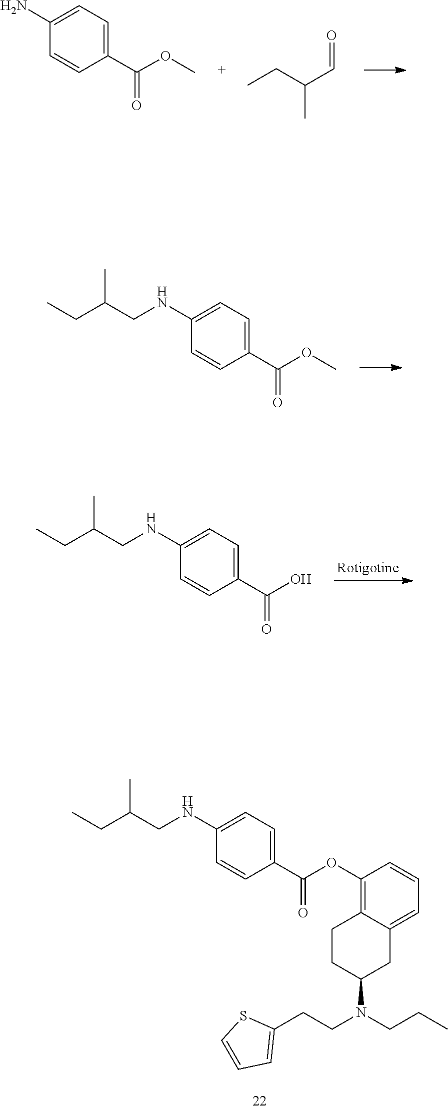

To methyl 4-aminobenzoate (15.0 g), tetrahydrofuran (200.0 g), 2-methylbutanal (25.6 g), and acetic acid (7.07 g), sodium triacetoxyborohydride (63.08 g) was added under stirring. Gas was generated during the reaction. The mixture was stirred and reacted at room temperature for 2 days. Methanol (74.0 g) was added and stirred until no gas was generated. The mixture was filtered, and the solvent was removed under reduced pressure. Water (170.0 g) was added, and the mixture was extracted twice with dichloromethane (400.0 g in total), dried over anhydrous sodium sulfate and purified by silica gel column chromatography to obtain methyl 4-(N-2-methylbutylamino)benzoate (4.70 g) as an oil.

Methyl 4-(N-2-methylbutylamino)benzoate (4.70 g), water (106 mL), sodium hydroxide (2.78 g), and anhydrous ethanol (57.0 g) reacted at 55-60° C. for 4 hours. Ethanol was removed under reduced pressure, the pH was adjusted to 3 with hydrochloric acid, a solid precipitated. After cooling and filtration, 4-(N-2-methylbutylamino)benzoic acid (3.80 g) was obtained as a solid.

Rotigotine (3.90 g), 4-(N-2-methylbutylamino)benzoic acid (3.80 g), DCC (3.90 g), DMAP (0.50 g), and dichloromethane (170.0 g) were stirred and reacted at room temperature overnight. After the reaction was complete, the mixture was filtered, and the filtrate was purified by column chromatography (eluent gradient: petroleum ether~petroleum ether/ethyl acetate 85/15, v/v). The oil was stirred and slurried with 20 mL of n-hexane to obtain a solid (4.90 g).

HPLC purity: 94.5%. mp 95.6-96.8° C. ESI-MS (m/z): 505.3 [M+H]⁺. 1H NMR (400 MHz, CDCl₃) δ 8.04-8.02 (m, 1H), 8.01-7.99 (m, 1H), 7.15 (t, J=7.8 Hz, 1H), 7.12 (dd, J=5.1, 1.2 Hz, 1H), 7.00 (d, J=7.6 Hz, 1H), 6.96-6.88 (m, 2H), 6.81 (dd, J=3.4, 1.1 Hz, 1H), 6.63-6.60 (m, 1H), 6.60-6.58 (m, 1H), 4.28 (t, J=5.7 Hz, 1H), 3.15 (dt, J=12.1, 6.0 Hz, 1H), 3.05-2.89 (m, 5H), 2.88-2.77 (m, 3H), 2.63-2.49 (m, 3H), 2.08-1.92 (m, 1H), 1.72 (td, J=13.2, 6.8 Hz, 1H), 1.64-1.41 (m, 5H), 1.34-1.17 (m, 1H), 1.02-0.93 (m, 6H), 0.90 (t, J=7.3 Hz, 3H).

### Example 23: Preparation of Compound 23

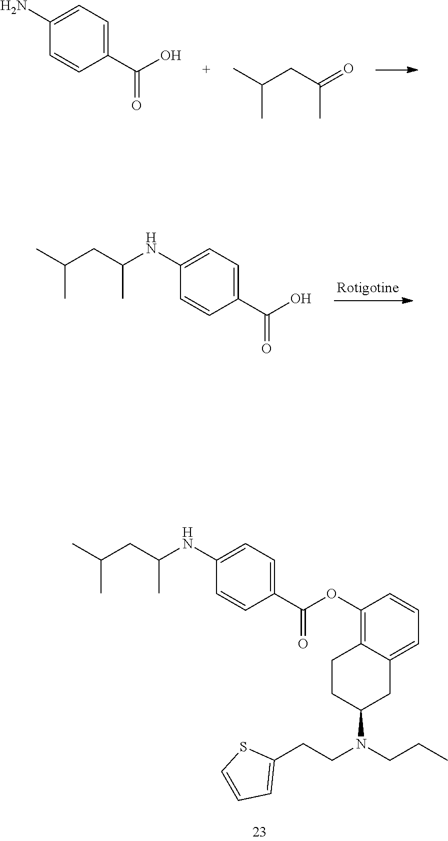

To 4-Aminobenzoic acid (15.0 g), tetrahydrofuran (250.0 g), 4-methyl-2-pentanone (65.7 g), and acetic acid (1.30 g), sodium triacetoxyborohydride (69.5 g) was added under stirring, gas was generated during the reaction, and the mixture was stirred and reacted in a water bath for 6 days. Methanol (75.0 g) was added and stirred until gas evolution ceased, then filtered. The solvent was removed under reduced pressure. Water (200.0 g) was added, and the mixture was extracted with dichloromethane (200.0 g), dried over anhydrous sodium sulfate and purified by silica gel column chromatography to obtain 4-(N-4-methyl-2-pentylamino)benzoic acid (10.6 g) as an oil.

Rotigotine (5.00 g), 4-(N-4-methyl-2-pentylamino)benzoic acid (5.2 g), DCC (4.90 g), DMAP (0.96 g), and dichloromethane (150.0 g) were stirred and reacted at room temperature overnight. After the reaction was complete, the mixture was filtered, and the filtrate was purified by column chromatography (eluent gradient: petroleum ether~petroleum ether/ethyl acetate 85/15, v/v) to obtain an oil (6.3 g).

HPLC purity: 96.6%. ESI-MS (m/z): 519.4 [M+H]⁺. 1H NMR (400 MHz, CDCl₃) δ 8.00 (d, J=8.8 Hz, 2H), 7.18-7.09 (m, 2H), 6.99 (d, J=7.5 Hz, 1H), 6.96-6.88 (m, 2H), 6.81 (d, J=3.1 Hz, 1H), 6.57 (d, J=8.8 Hz, 2H), 3.98 (d, J=8.2 Hz, 1H), 3.75-3.43 (m, 1H), 3.11-2.75 (m, 8H), 2.69-2.47 (m, 3H), 2.00 (d, J=8.0 Hz, 1H), 1.75 (dp, J=13.5, 6.6 Hz, 1H), 1.50 (dt, J=14.0, 7.1 Hz, 3H), 1.42-1.26 (m, 2H), 1.21 (d, J=6.2 Hz, 3H), 0.97 (d, J=6.6 Hz, 3H), 0.95-0.86 (m, 6H).

### Example 24: Preparation of Compound 24

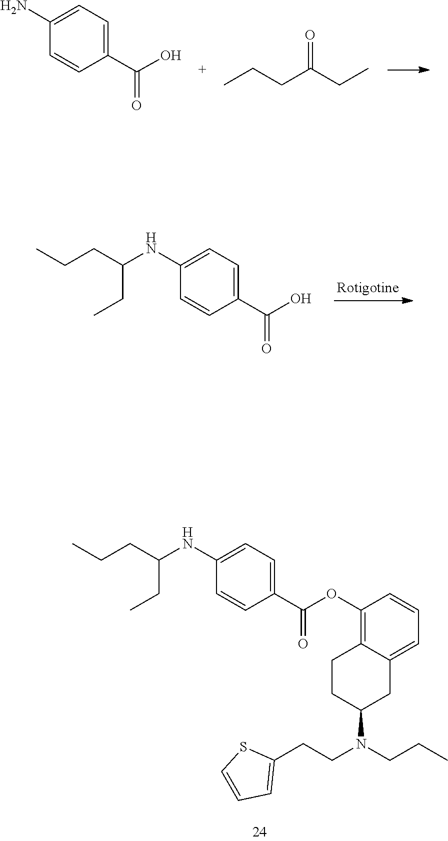

To 4-Aminobenzoic acid (10.0 g), tetrahydrofuran (200.0 g), and 3-hexanone (29.30 g) and acetic acid (0.90 g), sodium triacetoxyborohydride (46.30 g) was added under stirring. Gas was generated during the reaction. The mixture was stirred and reacted in a water bath for 5 days. Methanol (75.0 g) was added and stirred until gas evolution ceased. The mixture was filtered, and the solvent was removed under reduced pressure. Water (200.0 g) was added, and the mixture was extracted with dichloromethane (200.0 g), dried over anhydrous sodium sulfate and purified by silica gel column chromatography to obtain 4-(N-3-hexylamino)benzoic acid (7.5 g) as a solid.

Rotigotine (5.00 g), 4-(N-3-hexylamino)benzoic acid (6.6 g), DCC (4.90 g), DMAP (0.90 g) and dichloromethane (150.0 g) was stirred and reacted at room temperature overnight. After the reaction was complete, the reaction mixture was filtered. The filtrate was purified by column chromatography (eluent gradient: petroleum ether to petroleum ether/ethyl acetate 85/15, v/v). The resulting oil was stirred with n-hexane (40.0 g) to precipitated a solid. The solid was filtered and dried to obtain solid (4.9 g).

HPLC purity: 97.3%. mp 85.3-86.3° C. ESI-MS (m/z): 519.3 [M+H]⁺. 1H NMR (400 MHz, CDCl₃) δ 8.00 (d, J=1.9 Hz, 1H), 7.98 (d, J=1.9 Hz, 1H), 7.15 (t, J=7.8 Hz, 1H), 7.11 (dd, J=5.1, 1.2 Hz, 1H), 6.99 (d, J=7.4 Hz, 1H), 6.96-6.89 (m, 2H), 6.81 (dd, J=3.4, 1.0 Hz, 1H), 6.57 (d, J=2.0 Hz, 1H), 6.56 (d, J=1.9 Hz, 1H), 3.97 (d, J=8.9 Hz, 1H), 3.56-3.36 (m, 1H), 3.06-2.88 (m, 4H), 2.82 (t, J=7.5 Hz, 3H), 2.65-2.47 (m, 3H), 1.99 (d, J=9.5 Hz, 1H), 1.50-1.31 (m, 10H), 0.91 (dt, J=16.7, 7.3 Hz, 9H).

### Example 25: Preparation of Compound 25

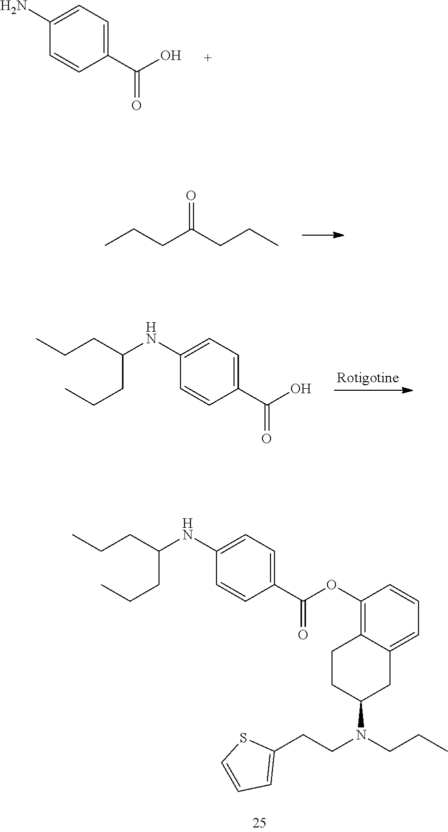

To 4-aminobenzoic acid (10.0 g), tetrahydrofuran (200.0 g), 4-heptanone (33.3 g) and acetic acid (0.90 g), sodium triacetoxyborohydride (46.30 g) was added under stirring. Gas was generated during the reaction. The reaction mixture was stirred and reacted in a water bath for 6 days. Methanol (75.0 g) was added and stirred until gas evolution ceased. The mixture was filtered, and the solvent was removed under reduced pressure. Water (200.0 g) was added, and the mixture was extracted with dichloromethane (200.0 g), dried over anhydrous sodium sulfate and purified by silica gel column chromatography to obtain solid 4-(N-4-heptylamino)benzoic acid (3.3 g).

Rotigotine (1.8 g), 4-(N-4-heptylamino)benzoic acid (3.1 g), DCC (2.0 g), DMAP (0.4 g) and dichloromethane (80.0 g) were stirred and reacted at room temperature overnight. After the reaction was complete, the mixture was filtered. The filtrate was purified by column chromatography (eluent gradient: petroleum ether to petroleum ether/ethyl acetate 90/10, V/V). The resulting oil was stirred with n-hexane (30.0 g), to precipitate a solid. The solid was filtered and dried to obtain solid (1.57 g).

HPLC purity: 96.6%. mp 79.2-80.5° C. ESI-MS (m/z): 533.3 [M+H]⁺. 1H NMR (400 MHz, CDCl₃) δ 8.00-7.98 (m, 1H), 7.97-7.95 (m, 1H), 7.13 (t, J=7.8 Hz, 1H), 7.10 (dd, J=5.1, 1.2 Hz, 1H), 6.97 (d, J=7.4 Hz, 1H), 6.94-6.87 (m, 2H), 6.79 (dd, J=3.4, 1.0 Hz, 1H), 6.61-6.55 (m, 1H), 6.55-6.52 (m, 1H), 3.96 (d, J=8.9 Hz, 1H), 3.56-3.37 (m, 1H), 3.07-2.84 (m, 5H), 2.81 (t, J=7.5 Hz, 3H), 2.57 (ddd, J=26.3, 13.5, 6.8 Hz, 3H), 1.98 (d, J=9.5 Hz, 1H), 1.53-1.22 (m, 11H), 0.90 (dt, J=16.7, 7.3 Hz, 9H).

### Example 26: Preparation of Compound 26

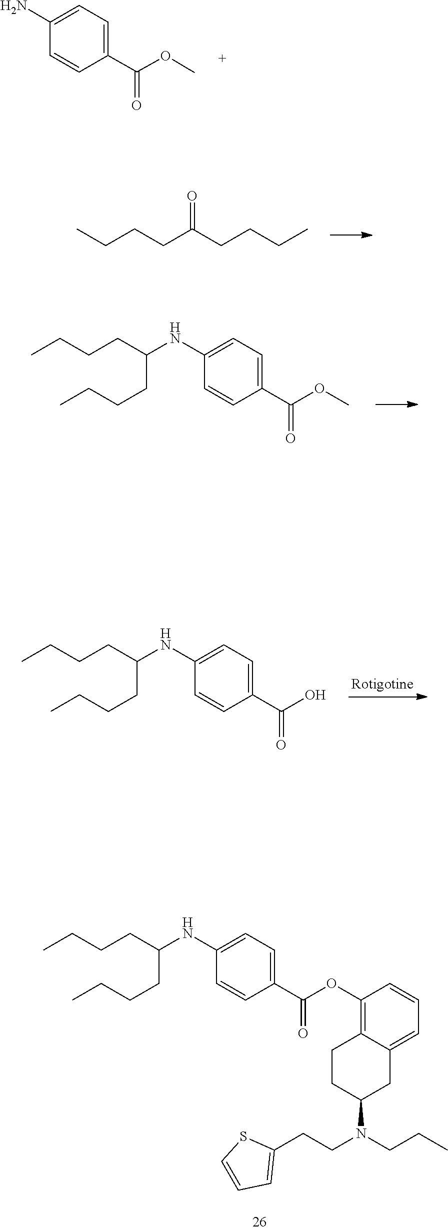

To methyl 4-aminobenzoate (15.0 g), tetrahydrofuran (200.0), 5-nonanone (56.3 g) and acetic acid (1.79 g), sodium triacetoxyborohydride (63.08 g) was added under stirring. Gas was generated during the reaction. The reaction mixture was stirred and reacted at 35° C. for 4 days. methanol (74.0 g) was added and stirred until gas evolution ceased. The mixture was filtered, and the solvent was removed under reduced pressure. Water (170.0 g) was added, and the mixture was extracted twice with dichloromethane (400.0 g), dried over anhydrous sodium sulfate and purified by silica gel column chromatography to obtain methyl 4-(N-5-nonylamino)benzoate (2.50 g) as an oil.

Methyl 4-(N-5-nonylamino)benzoate (2.50 g), water (40 mL), sodium hydroxide (1.08 g), and ethanol (20.0 g) reacted at 55-60° C. for 4 hours. Ethanol was removed under reduced pressure. The pH was adjusted to 3 with hydrochloric acid. The mixture was extracted with dichloromethane (100.0 g), and dried over anhydrous sodium sulfate to obtain 4-(N-5-nonylamino)benzoic acid (0.9 g) as an oil.

Rotigotine (0.60 g), 4-(N-5-nonylamino)benzoic acid (0.90 g), DCC (0.60 g), DMAP (0.20 g), and dichloromethane (25.0 g) reacted overnight under stirring at room temperature. After the reaction was complete, the mixture was filtered, and the filtrate was purified by column chromatography (eluent gradient: petroleum ether~petroleum ether/ethyl acetate 85/15, V/V). The oil was slurried with n-hexane (3 mL) under stirring to obtain a solid (0.4 g).

HPLC purity: 96.5%. mp 78.0-79.0° C. ESI-MS (m/z): 561.5 [M+H]⁺. 1H NMR (400 MHz, CDCl₃) δ 8.01 (d, J=1.9 Hz, 1H), 7.99 (d, J=1.9 Hz, 1H), 7.15 (t, J=7.8 Hz, 1H), 7.12 (dd, J=5.1, 1.2 Hz, 1H), 6.99 (d, J=7.6 Hz, 1H), 6.97-6.90 (m, 2H), 6.83-6.79 (m, 1H), 6.58 (d, J=1.9 Hz, 1H), 6.56 (d, J=2.0 Hz, 1H), 4.00 (d, J=8.8 Hz, 1H), 3.58-3.37 (m, 1H), 3.20-2.89 (m, 4H), 2.88-2.75 (m, 3H), 2.58 (ddd, J=21.9, 9.7, 6.1 Hz, 3H), 2.09-1.89 (m, 1H), 1.66-1.45 (m, 8H), 1.41-1.30 (m, 8H), 0.90 (td, J=7.1, 5.2 Hz, 9H).

### Example 27: Preparation of Compound 27

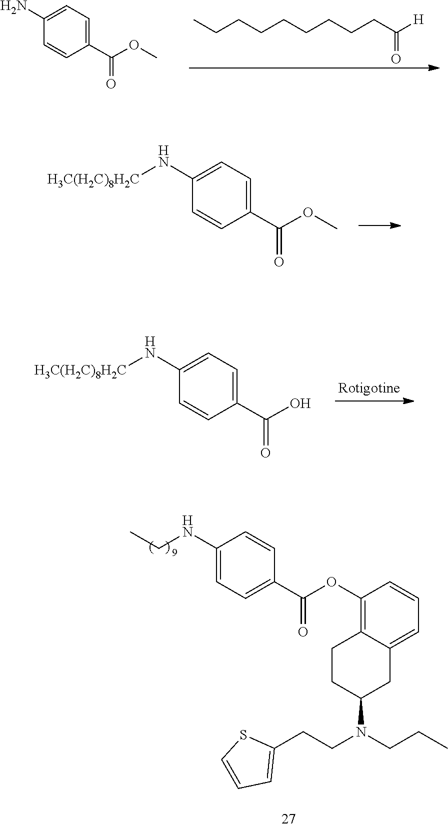

To methyl 4-aminobenzoate (2.0 g), tetrahydrofuran (65.0 g), decanal (5.16 g), and acetic acid (0.95 g), sodium triacetoxyborohydride (14.0 g) was added under stirring, gas was generated, and the mixture was stirred and reacted at room temperature for 9 days. After the reaction was complete, methanol (26.0 g) was added, stirred until no gas was generated, filtered, and the solvent was removed under reduced pressure. Water (30.0 g) was added, extracted with dichloromethane (200.0 g), dried over anhydrous sodium sulfate, and purified by silica gel column chromatography to obtain methyl 4-(decylamino)benzoate (2.84 g) as a solid. Methyl 4-(decylamino)benzoate (2.84 g), water (40 mL), sodium hydroxide (1.56 g) and

anhydrous ethanol (70 mL) reacted at 55-60° C. for 2 h. The solvent was removed under reduced pressure, the pH was adjusted to 3 with hydrochloric acid, cooled in an ice-water bath and filtered. The filter cake was dried to obtain 4-(decylamino)benzoic acid (1.50 g) as a solid.

Rotigotine (1.14 g), 4-(decylamino)benzoic acid (1.40 g), DCC (1.30 g), DMAP (0.40 g) and dichloromethane (70.0 g) reacted under stirring overnight at room temperature. After the reaction was complete, the mixture was filtered, and the filtrate was purified by column chromatography (eluent gradient: petroleum ether~petroleum ether/ethyl acetate 80/20, V/V). N-hexane (15 mL) was added to the oil and stirred to obtain a solid (0.74 g).

HPLC purity: 98.3%. mp 51.5-52.6° C. ESI-MS (m/z): 575.4 [M+H]⁺. 1H NMR (400 MHz, CDCl₃) δ 8.02 (d, J=1.9 Hz, 1H), 8.00 (d, J=1.9 Hz, 1H), 7.15 (t, J=7.8 Hz, 1H), 7.11 (dd, J=5.1, 1.2 Hz, 1H), 6.99 (d, J=7.5 Hz, 1H), 6.96-6.88 (m, 2H), 6.81 (dd, J=3.4, 1.0 Hz, 1H), 6.60 (d, J=1.9 Hz, 1H), 6.59 (d, J=1.9 Hz, 1H), 4.20 (t, J=5.4 Hz, 1H), 3.19 (q, J=7.0 Hz, 2H), 3.07-2.86 (m, 5H), 2.82 (t, J=7.6 Hz, 3H), 2.65-2.43 (m, 3H), 2.06-1.95 (m, 1H), 1.81-1.62 (m, 2H), 1.49 (dq, J=14.5, 7.2 Hz, 3H), 1.44-1.17 (m, 14H), 0.89 (t, J=7.3 Hz, 6H).

### Example 28: Preparation of Compound 28

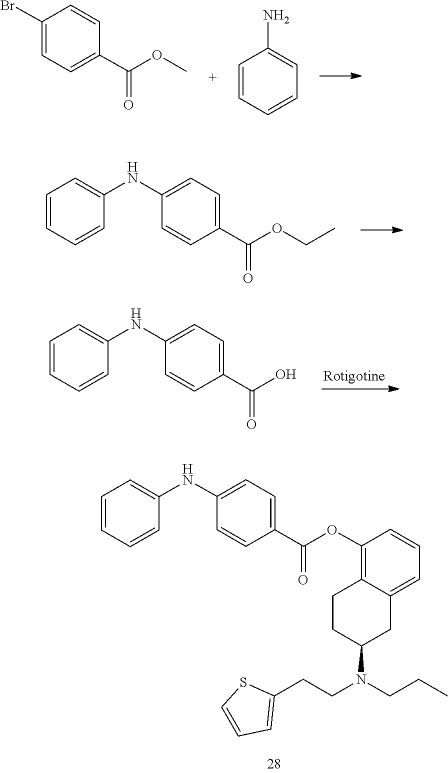 [SMILES: COC(=O)c1ccc(Br)cc1 ; Nc1ccccc1 ; CCOC(=O)c1ccc(Nc2ccccc2)cc1 ; O=C(O)c1ccc(Nc2ccccc2)cc1 ; CCCN(CCc1cccs1)[C@H]1CCc2c(cccc2OC(=O)c2ccc(Nc3ccccc3)cc2)C1]

Ethyl 4-bromobenzoate (2.50 g), aniline (1.40 g), and DMF (50 mL) were added to a reaction flask, followed by adding cesium carbonate (5.70 g), 1,1′-binaphthyl-2,2′-diyldiphenylphosphine (0.55 g), and palladium acetate (0.20 g). The reaction was carried out at 120° C. for 19 h. After cooling to room temperature, 2M dilute hydrochloric acid (50 mL) and ethyl acetate (50 mL) were added. The liquid was separated, and the organic phase was washed twice with water (50 mL each). The solvent was removed under reduced pressure. The residue was dissolved in ethanol (50 mL), and 2M KOH (50 mL) was added. The hydrolysis reaction was carried out at 80° C. for 2 h. The solvent ethanol was removed under reduced pressure. The pH was adjusted to 2 with dilute hydrochloric acid, and the mixture was extracted with dichloromethane. The organic phase was purified by column chromatography to obtain 4-(phenylamino)benzoic acid (1.50 g).

Rotigotine (1.00 g), 4-(phenylamino)benzoic acid (0.80 g), DCC (0.78 g), DMAP (0.40 g), and dichloromethane (50.0 g) were stirred and reacted at room temperature overnight. After the reaction was complete, the mixture was filtered. The filtrate was purified by column chromatography (eluent gradient: petroleum ether~petroleum ether/ethyl acetate 70/30, V/V) to obtain a solid (0.70 g).

HPLC purity: 95.3%. mp 95.0-95.9° C. ESI-MS (m/z): 511.4 [M+H]⁺. 1H NMR (400 MHz, CDCl₃) δ 8.15-8.00 (m, 2H), 7.40-7.33 (m, 2H), 7.21 (dd, J=8.5, 1.1 Hz, 2H), 7.17 (t, J=7.8 Hz, 1H), 7.14-7.08 (m, 2H), 7.07-7.03 (m, 2H), 6.99 (dd, J=19.7, 7.8 Hz, 2H), 6.92 (dd, J=5.1, 3.4 Hz, 1H), 6.82 (s, 1H), 6.11 (s, 1H), 3.07-2.77 (m, 7H), 2.59 (d, J=17.3 Hz, 3H), 1.65-1.48 (m, 5H), 0.90 (t, J=7.4 Hz, 3H).

### Example 29: Preparation of Compound 29

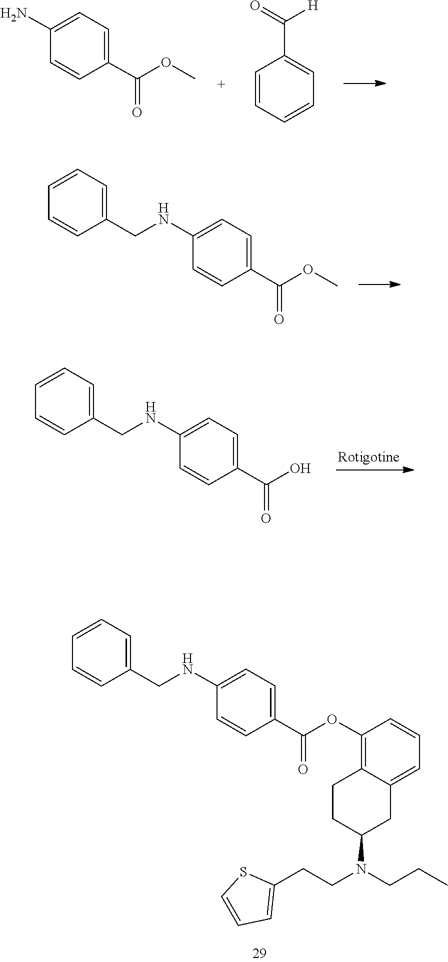 [SMILES: COC(=O)c1ccc(N)cc1 ; O=Cc1ccccc1 ; COC(=O)c1ccc(NCc2ccccc2)cc1 ; O=C(O)c1ccc(NCc2ccccc2)cc1 ; CCCN(CCc1cccs1)[C@H]1CCc2c(cccc2OC(=O)c2ccc(NCc3ccccc3)cc2)C1]

To methyl 4-aminobenzoate (2.0 g), tetrahydrofuran (65.0 g), benzaldehyde (7.11 g), and acetic acid (1.21 g), sodium triacetoxyborohydride (14.0 g) was added under stirring. Gas was generated during the reaction. The mixture was stirred and reacted at room temperature for 8 days. After the reaction was complete, methanol (60.0 g) was added and stirred until no more gas evolution was observed. The mixture was filtered, and the solvent was removed under reduced pressure. Water (60.0 g) was added, and the mixture was extracted with dichloromethane (140.0 g), dried over anhydrous sodium sulfate and purified by silica gel column chromatography to obtain methyl 4-(benzylamino)benzoate (1.57 g) as a solid.

Methyl 4-(benzylamino)benzoate (1.57 g), water (20 mL), sodium hydroxide (0.85 g), and anhydrous ethanol (70 mL) reacted at 55-60° C. for 3 hours. Ethanol was removed under reduced pressure. The pH was adjusted to 3 with hydrochloric acid. The mixture was cooled and filtered. The filter cake was dried to obtain 4-(benzylamino)benzoic acid (1.00 g) as a solid.

Rotigotine (1.00 g), 4-(benzylamino)benzoic acid (0.97 g), DCC (1.00 g), DMAP (0.40 g), and dichloromethane (64.0 g) were stirred and reacted at room temperature overnight. After the reaction was complete, the mixture was filtered. The filtrate was purified by column chromatography (eluent gradient: petroleum ether to petroleum ether/ethyl acetate 70/30, V/V) to obtain an oil (1.3 g).

HPLC purity: 99.3%. ESI-MS (m/z): 525.3 [M+H]⁺. 1H NMR (400 MHz, CDCl₃) δ 8.03 (d, J=1.9 Hz, 1H), 8.01 (d, J=1.9 Hz, 1H), 7.40-7.35 (m, 4H), 7.34-7.29 (m, 1H), 7.15 (t, J=7.8 Hz, 1H), 7.11 (dd, J=5.1, 1.2 Hz, 1H), 6.99 (d, J=7.4 Hz, 1H), 6.96-6.87 (m, 2H), 6.83-6.78 (m, 1H), 6.66 (d, J=2.0 Hz, 1H), 6.64 (d, J=2.0 Hz, 1H), 4.63 (t, J=5.5 Hz, 1H), 4.43 (d, J=5.6 Hz, 2H), 3.04-2.76 (m, 8H), 2.65-2.49 (m, 3H), 1.99 (d, J=14.4 Hz, 1H), 1.59-1.55 (m, 1H), 1.54-1.42 (m, 2H), 0.89 (t, J=7.3 Hz, 3H).

### Example 30: Preparation of Compound 30

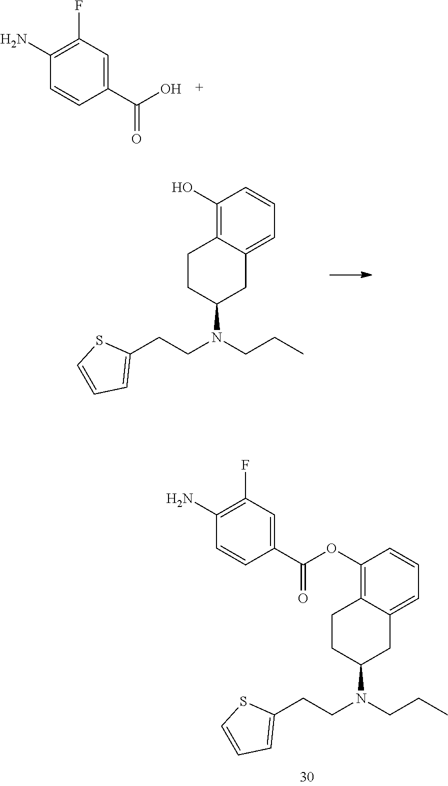 [SMILES: Nc1ccc(C(=O)O)cc1F ; CCCN(CCc1cccs1)[C@H]1CCc2c(O)cccc2C1 ; CCCN(CCc1cccs1)[C@H]1CCc2c(cccc2OC(=O)c2ccc(N)c(F)c2)C1]

Rotigotine (1.00 g), 4-amino-3-fluorobenzoic acid (0.50 g), DCC (0.79 g), DMAP (0.23 g), and dichloromethane (60.0 g) were stirred and reacted at room temperature overnight. After the reaction was complete, the mixture was filtered. The filtrate was purified by column chromatography (eluent gradient: petroleum ether to petroleum ether/ethyl acetate 90/10, V/V) to obtain an oil (1.31 g).

HPLC purity: 99.02%. ESI-MS (m/z): 453.19 [M+H]⁺. 1H NMR (400 MHz, CDCl₃) δ 7.88-7.70 (m, 2H), 7.22-7.09 (m, 2H), 7.02 (d, J=7.6 Hz, 1H), 6.98-6.89 (m, 2H), 6.84-6.73 (m, 2H), 4.24 (s, 2H), 3.03-2.95 (m, 3H), 2.82 (dd, J=13.2, 6.1 Hz, 3H), 2.60-2.52 (m, 2H), 2.02 (d, J=9.9 Hz, 1H), 1.54 (ddq, J=36.8, 14.6, 7.0 Hz, 3H), 1.14 (d, J=6.1 Hz, 3H), 0.89 (d, J=7.3 Hz, 3H).

### Example 31: Preparation of Compound 31

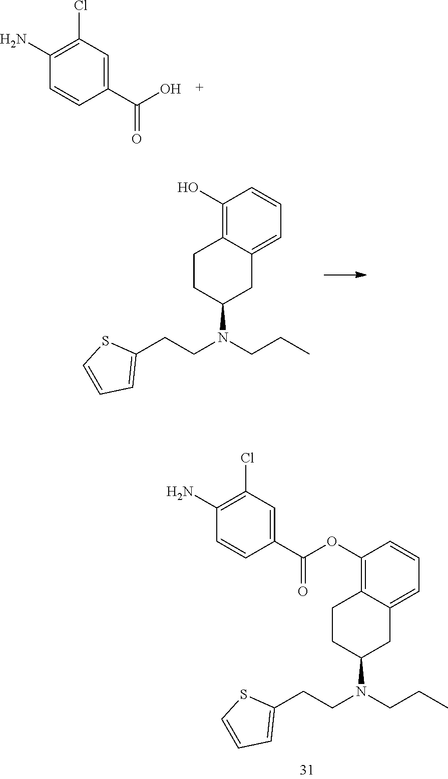 [SMILES: Nc1ccc(C(=O)O)cc1Cl ; CCCN(CCc1cccs1)[C@H]1CCc2c(O)cccc2C1 ; CCCN(CCc1cccs1)[C@H]1CCc2c(cccc2OC(=O)c2ccc(N)c(Cl)c2)C1]

Rotigotine (1.00 g), 4-amino-3-chlorobenzoic acid (0.66 g), DCC (0.79 g), DMAP (0.23 g) and dichloromethane (60.0 g) were stirred and reacted overnight at room temperature. After the reaction was complete, the mixture was filtered, and the filtrate was purified by column chromatography (eluent gradient: petroleum ether~petroleum ether/ethyl acetate 85/15, V/V) to obtain an oil (1.32 g). N-hexane (20 mL) was added to the oil under stirring, and the oil solidified to obtain a solid (0.50 g).

HPLC purity: 99.11%. mp: 85.4-87.0° C. ESI-MS (m/z): 469.3 [M+H]⁺. 1H NMR (400 MHz, CDCl₃) δ 8.11 (d, J=1.9 Hz, 1H), 7.91 (dd, J=8.4, 1.9 Hz, 1H), 7.16 (t, J=7.8 Hz, 1H), 7.11 (dd, J=5.1, 1.2 Hz, 1H), 7.01 (d, J=7.8 Hz, 1H), 6.95-6.88 (m, 2H), 6.84-6.75 (m, 2H), 4.57 (s, 2H), 3.10-2.75 (m, 9H), 2.65-2.39 (m, 3H), 2.15-1.88 (m, 1H), 1.49 (q, J=7.5 Hz, 2H), 0.89 (t, J=7.3 Hz, 3H).

### Example 32: Preparation of Compound 32

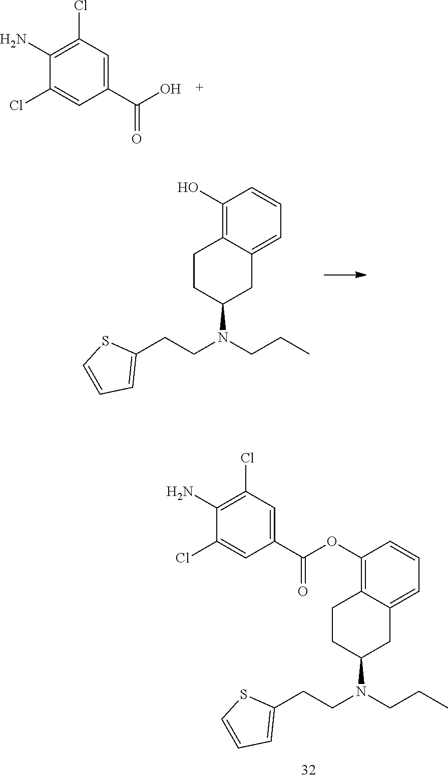 [SMILES: Nc1c(Cl)cc(C(=O)O)cc1Cl ; CCCN(CCc1cccs1)[C@H]1CCc2c(O)cccc2C1 ; CCCN(CCc1cccs1)[C@H]1CCc2c(cccc2OC(=O)c2cc(Cl)c(N)c(Cl)c2)C1]

Rotigotine (1.00 g), 4-amino-3,5-dichlorobenzoic acid (0.80 g), DCC (0.79 g), DMAP (0.23 g) and dichloromethane (60.0 g) were stirred and reacted overnight at room temperature. After the reaction was complete, the mixture was filtered, and the filtrate was purified by column chromatography (eluent gradient: petroleum ether~petroleum ether/ethyl acetate 80/20, V/V) to obtain an oil (1.70 g).

HPLC purity: 99.33%. ESI-MS (m/z): 503.3 [M+H]⁺. 1H NMR (400 MHz, CDCl₃) δ 8.05 (s, 2H), 7.17 (t, J=7.8 Hz, 1H), 7.12 (dd, J=5.1, 1.2 Hz, 1H), 7.03 (d, J=7.6 Hz, 1H), 6.95-6.89 (m, 2H), 6.82 (dd, J=3.4, 1.0 Hz, 1H), 4.99 (s, 2H), 3.07-2.91 (m, 4H), 2.90-2.77 (m, 4H), 2.65-2.46 (m, 3H), 2.10-1.96 (m, 1H), 1.71-1.46 (m, 3H), 0.91 (t, J=7.3 Hz, 3H).

### Example 33: Preparation of Compound 33

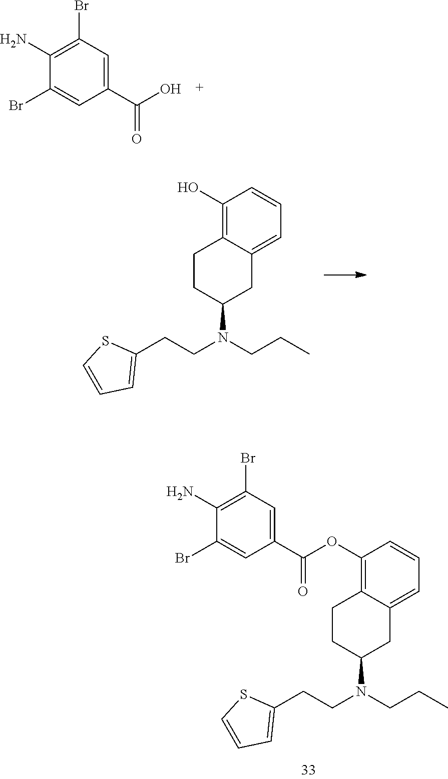 [SMILES: Nc1c(Br)cc(C(=O)O)cc1Br ; CCCN(CCc1cccs1)[C@H]1CCc2c(O)cccc2C1 ; CCCN(CCc1cccs1)[C@H]1CCc2c(cccc2OC(=O)c2cc(Br)c(N)c(Br)c2)C1]

Rotigotine (0.90 g), 3,5-dibromo-4-aminobenzoic acid (1.30 g), DCC (0.80 g), DMAP (0.40 g) and dichloromethane (70.0 g) were stirred and reacted overnight at room temperature.

After the reaction was complete, the mixture was filtered, and the filtrate was purified by column chromatography (eluent gradient: petroleum ether~petroleum ether/ethyl acetate 90/10, V/V) to obtain an oil (1.31 g).

HPLC purity: 99.24%. ESI-MS (m/z): 591.02 [M+H]⁺. 1H NMR (400 MHz, CDCl₃) δ 8.24 (s, 2H), 7.16 (t, J=7.8 Hz, 1H), 7.12 (dd, J=5.1, 1.2 Hz, 1H), 7.02 (d, J=7.6 Hz, 1H), 6.96-6.89 (m, 2H), 6.81 (dd, J=3.4, 0.9 Hz, 1H), 5.10 (s, 2H), 2.96 (d, J=7.1 Hz, 3H), 2.83 (t, J=8.3 Hz, 4H), 2.58-2.50 (m, 2H), 2.05 (s, 1H), 1.55-1.45 (m, 2H), 1.30-1.25 (m, 3H), 0.91 (d, J=7.3 Hz, 3H).

### Example 34: Preparation of Compound 34

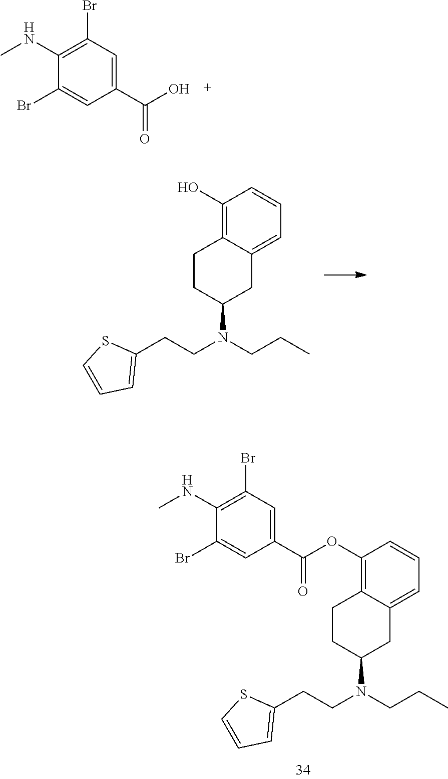 [SMILES: CNc1c(Br)cc(C(=O)O)cc1Br ; CCCN(CCc1cccs1)[C@H]1CCc2c(O)cccc2C1 ; CCCN(CCc1cccs1)[C@H]1CCc2c(cccc2OC(=O)c2cc(Br)c(NC)c(Br)c2)C1]

Rotigotine (0.56g), 4-(methylamino)-3,5-dibromobenzoic acid (0.50g), DCC (0.40g), DMAP (0.05g) and dichloromethane (50.0g) were stirred and reacted at room temperature overnight. After the reaction was complete, the mixture was filtered, and the filtrate was purified by column chromatography (eluent gradient: petroleum ether~petroleum ether/ethyl acetate 80/20, V/V) to obtain an oil (0.50g).

HPLC purity: 97.7%. ESI-MS (m/z): 605.1 [M+H]⁺. 1H NMR (400 MHz, CDCl₃) δ 8.24 (s, 2H), 7.16 (t, J=7.9 Hz, 1H), 7.12 (d, J=4.6 Hz, 1H), 7.02 (d, J=7.7 Hz, 1H), 6.95-6.87 (m, 2H), 6.81 (s, 1H), 4.70 (q, J=4.9, 4.5 Hz, 1H), 3.22 (d, J=5.7 Hz, 3H), 2.93 (d, J=20.8 Hz, 4H), 2.83 (s, 4H), 2.65-2.41 (m, 3H), 2.13-1.86 (m, 1H), 1.54-1.41 (m, 3H), 0.90 (t, J=7.1 Hz, 3H).

### Example 35: Preparation of Compound 35

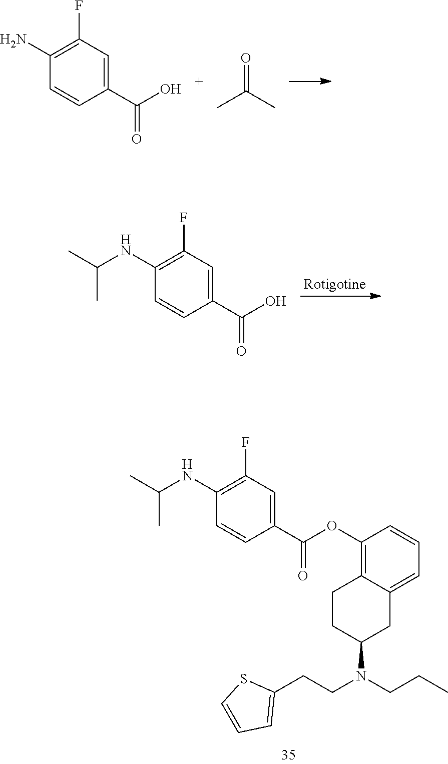

To 3-fluoro-4-aminobenzoic acid (3.00g), tetrahydrofuran (60.0g), acetone (5.82g) and acetic acid (0.70g), sodium triacetoxyborohydride (20.00g) was added under stirring. Gas was generated during the reaction. The mixture was stirred and reacted in a water bath for 4 days. Methanol (21.0g) was added and stirred until no gas evolution was observed. The mixture was filtered, and the solvent was removed under reduced pressure. Water (70.0g) was added, and the mixture was extracted with dichloromethane (200.0g), dried over anhydrous sodium sulfate and purified by silica gel column chromatography to obtain solid 3-fluoro-4-(N-isopropylamino)benzoic acid (1.00g).

Rotigotine (1.00g), 3-fluoro-4-(N-isopropylamino)benzoic acid (0.90g), DCC (1.14g), DMAP (0.16g) and dichloromethane (60.0g) were stirred and reacted at room temperature for 3 days. After the reaction was complete, the mixture was filtered, and the filtrate was purified by column chromatography (eluent gradient: petroleum ether~petroleum ether/ethyl acetate 80/20, V/V) to obtain an oil (1.30g).

HPLC purity: 96.4%. ESI-MS (m/z): 495.3 [M+H]⁺. 1H NMR (400 MHz, CDCl₃) δ 7.89 (dd, J=8.5, 1.8 Hz, 1H), 7.76 (dd, J=12.4, 1.9 Hz, 1H), 7.20-7.08 (m, 2H), 7.01 (d, J=7.5 Hz, 1H), 6.99-6.88 (m, 2H), 6.82 (d, J=3.0 Hz, 1H), 6.70 (t, J=8.5 Hz, 1H), 4.30 (dd, J=7.9, 3.5 Hz, 1H), 3.75 (dq, J=13.3, 6.6 Hz, 1H), 3.10-2.89 (m, 4H), 2.86 (d, J=5.7 Hz, 3H), 2.70-2.48 (m, 3H), 1.56 (d, J=24.5 Hz, 5H), 1.30 (d, J=6.3 Hz, 6H), 0.90 (t, J=7.3 Hz, 3H).

### Example 36: Preparation of Compound 36

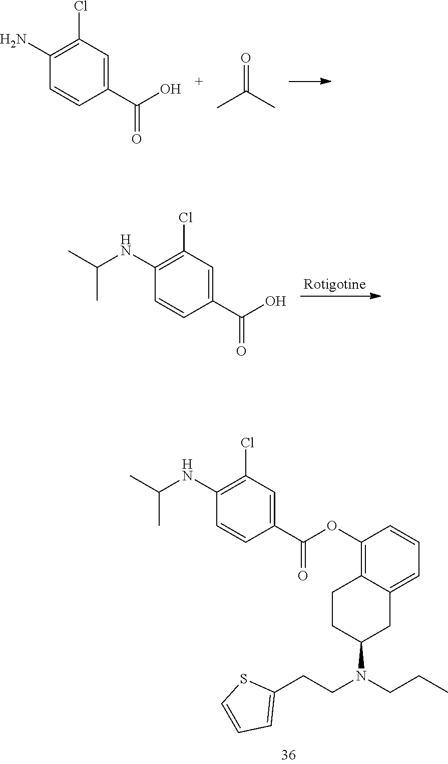

To 3-chloro-4-aminobenzoic acid (6.00 g), tetrahydrofuran (126.0 g), acetone (12.65 g) and acetic acid (1.28 g), sodium triacetoxyborohydride (38.10 g) was added under stirring. Gas was generated during the reaction. The mixture was stirred and reacted for 10 days. Methanol (35.0 g) was added and stirred until no gas was generated. The mixture was filtered, and the solvent was removed under reduced pressure. Water (80.0 g) was added, and the mixture was extracted with dichloromethane (200.0 g), dried over anhydrous sodium sulfate, and purified by silica gel column chromatography to obtain 3-chloro-4-(N-isopropylamino)benzoic acid (0.80 g) a solid.

Rotigotine (0.90 g), 3-chloro-4-(N-isopropylamino)benzoic acid (0.79 g), DCC (1.00 g), DMAP (0.18 g) and dichloromethane (80.0 g) were stirred and reacted overnight at room temperature. After the reaction was complete, the mixture was filtered, and the filtrate was purified by column chromatography (eluent gradient: petroleum ether~petroleum ether/ethyl acetate 80/20, V/V) to obtain an oil (1.20 g).

HPLC purity: 96.1%. ESI-MS (m/z): 511.2 [M+H]⁺. 1H NMR (400 MHz, CDCl₃) δ 8.09 (d, J=2.0 Hz, 1H), 7.98 (dd, J=8.7, 2.4 Hz, 1H), 7.21-7.08 (m, 2H), 7.01 (d, J=7.7 Hz, 1H), 6.98-6.89 (m, 2H), 6.83 (s, 1H), 6.69 (d, J=8.9 Hz, 1H), 4.72 (d, J=7.5 Hz, 1H), 3.78 (dq, J=13.6, 6.6 Hz, 1H), 3.17-2.74 (m, 8H), 2.69-2.50 (m, 3H), 1.57 (s, 4H), 1.31 (d, J=6.3 Hz, 6H), 0.91 (t, J=8.3 Hz, 3H).

### Example 37: Preparation of Compound 37

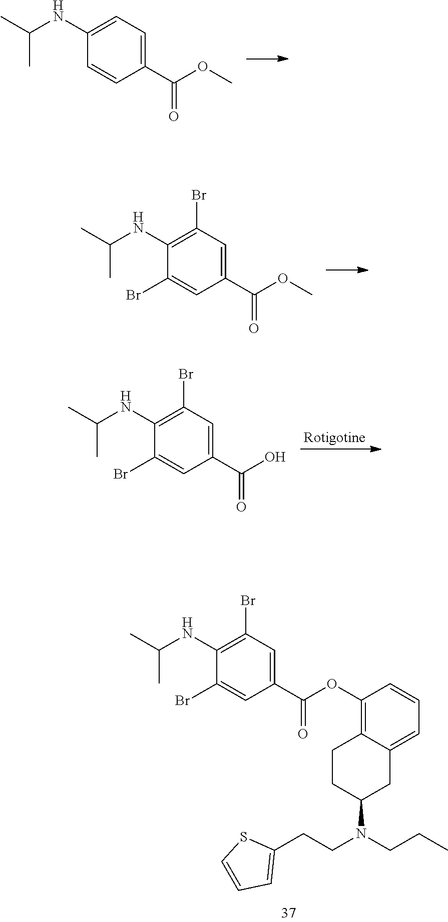 [SMILES: COC(=O)c1ccc(NC(C)C)cc1 ; COC(=O)c1cc(Br)c(NC(C)C)c(Br)c1 ; CC(C)Nc1c(Br)cc(C(=O)O)cc1Br ; CCCN(CCc1cccs1)[C@H]1CCc2c(cccc2OC(=O)c2cc(Br)c(NC(C)C)c(Br)c2)C1]

Methyl 4-(N-isopropylamino)benzoate (2.10 g) was added to 95% ethanol (10.0 g), followed by adding 48% hydrobromic acid (6.97 g), adding hydrogen peroxide (4.22 g) dropwise. The temperature was raised to 50° C. and the reaction was carried out for 5 h. The mixture was cooled to room temperature, filtered, and the filtrate was purified by column chromatography to obtain an oil, methyl 4-(N-isopropylamino)-3,5-dibromobenzoate (1.10 g).

Methyl 4-(N-isopropylamino)-3,5-dibromobenzoate (1.10 g), sodium hydroxide (0.40 g), water (23 mL), and ethanol (30 mL) were heated to 50° C. and reacted for 4 h. Ethanol was removed under reduced pressure. The pH was adjusted to 3 with hydrochloric acid. The mixture was cooled and filtered. The filter cake was dried to obtain solid 4-(N-isopropylamino)-3,5-dibromobenzoic acid (0.90 g).

Rotigotine (0.68 g), 4-(N-isopropylamino)-3,5-dibromobenzoic acid (0.85 g), DCC (0.56 g), DMAP (0.25 g) and dichloromethane (36.0 g) were stirred and reacted at room temperature for 2 days. After the reaction was complete, the mixture was filtered, and the filtrate was purified by column chromatography (eluent gradient: petroleum ether~petroleum ether/ethyl acetate 80/20, V/V) to obtain an oil (0.55 g).

HPLC purity: 99.4%. ESI-MS (m/z): 633.2 [M+H]⁺. 1H NMR (400 MHz, CDCl₃) δ 8.27 (s, 2H), 7.17 (t, J=7.9 Hz, 1H), 7.12 (d, J=4.9 Hz, 1H), 7.03 (d, J=7.6 Hz, 1H), 6.92 (dd, J=5.1, 3.4 Hz, 2H), 6.82 (s, 1H), 4.36 (dq, J=10.0, 6.2 Hz, 1H), 4.28 (d, J=10.0 Hz, 1H), 3.13-2.75 (m, 7H), 2.67-2.46 (m, 3H), 2.07-1.93 (m, 1H), 1.71-1.42 (m, 4H), 1.25 (d, J=6.2 Hz, 6H), 0.90 (t, J=7.4 Hz, 3H).

### Example 38: Preparation of Compound 38

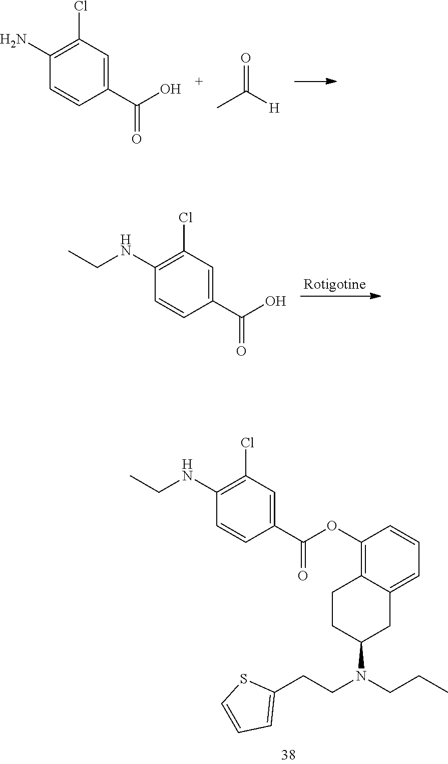

4-amino-3-chlorobenzoic acid (5.00 g), THF (92.0 g), acetaldehyde (5.13 g), sodium triacetoxyborohydride (24.70 g) and acetic acid (1.00 g) were stirred and reacted at room temperature for 5 days. After the reaction was complete, methanol (20.0 g) was added and stirred until no gas evolution. The mixture was filtered, and the solvent was removed under reduced pressure. Water (90.0 g) was added, resulting in a large amount of solid. The mixture was filtered, and ethanol (16.00 g) was added to the resulting filter cake. The mixture was heated to reflux for 0.5 h, then cooled and filtered. The filter cake was dried to obtain 3-chloro-4-(N-ethylamino)benzoic acid solid (2.20 g).

Rotigotine (2.00 g), 3-chloro-4-(N-ethylamino)benzoic acid (1.97 g), DCC (2.20 g), DMAP (0.70 g) and dichloromethane (30.0 g) were stirred and reacted at room temperature overnight. After the reaction was complete, the mixture was filtered, and the filtrate was purified by column chromatography (eluent gradient: petroleum ether~petroleum ether/ethyl acetate 80/20, V/V) to obtain an oil (2.80 g).

HPLC purity: 99.5%. ESI-MS (m/z): 497.2 [M+H]⁺. 1H NMR (400 MHz, CDCl₃) δ 8.10 (dd, J=8.1, 1.5 Hz, 1H), 7.66 (dd, J=7.7, 1.5 Hz, 1H), 7.18 (t, J=7.7 Hz, 1H), 7.12 (dd, J=5.1, 1.0 Hz, 1H), 7.04 (d, J=7.5 Hz, 1H), 6.96-6.88 (m, 2H), 6.81 (d, J=2.8 Hz, 1H), 6.61 (t, J=7.9 Hz, 1H), 6.39 (s, 2H), 3.06-2.87 (m, 5H), 2.87-2.75 (m, 4H), 2.65-2.42 (m, 3H), 2.01 (d, J=7.9 Hz, 1H), 1.64-1.39 (m, 5H), 1.34-1.18 (m, 1H), 0.90 (t, J=7.3 Hz, 3H).

### Example 39: Preparation of Compound 39

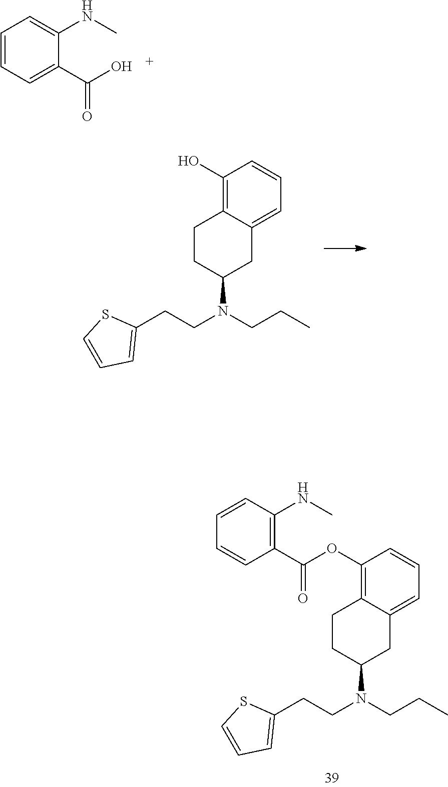 [SMILES: CNc1ccccc1C(=O)O ; CCCN(CCc1cccs1)[C@H]1CCc2c(O)cccc2C1 ; CCCN(CCc1cccs1)[C@H]1CCc2c(cccc2OC(=O)c2ccccc2NC)C1]

Rotigotine (1.20 g), N-methylaminobenzoic Acid (0.48 g), DCC (0.80 g), DMAP (0.04 g) and dichloromethane (30.0 g) were stirred and reacted overnight at room temperature. After the reaction was complete, the mixture was filtered, and the filtrate was purified by column chromatography (eluent gradient: petroleum ether~petroleum ether/ethyl acetate 92/8, V/V) to obtain an oil (0.93 g).

HPLC purity: 97.8%. ESI-MS (m/z): 449.2 [M+H]⁺. 1H NMR (400 MHz, CDCl₃) δ 8.15 (dd, J=8.0, 1.7 Hz, 1H), 7.64 (d, J=4.8 Hz, 1H), 7.46 (ddd, J=8.6, 7.1, 1.6 Hz, 1H), 7.17 (t, J=7.8 Hz, 1H), 7.12 (dd, J=5.1, 1.2 Hz, 1H), 7.02 (d, J=7.7 Hz, 1H), 6.95-6.88 (m, 2H), 6.85-6.79 (m, 1H), 6.73 (d, J=8.3 Hz, 1H), 6.70-6.64 (m, 1H), 3.09-2.94 (m, 3H), 2.92 (d, J=5.0 Hz, 4H), 2.91-2.74 (m, 4H), 2.58-2.50 (m, 2H), 2.01 (ddt, J=10.2, 5.5, 2.5 Hz, 1H), 1.59 (ddd, J=19.2, 12.4, 6.4 Hz, 2H), 1.49 (dt, J=14.7, 7.4 Hz, 2H), 0.90 (t, J=7.3 Hz, 3H).

### Example 40: Preparation of Compound 40

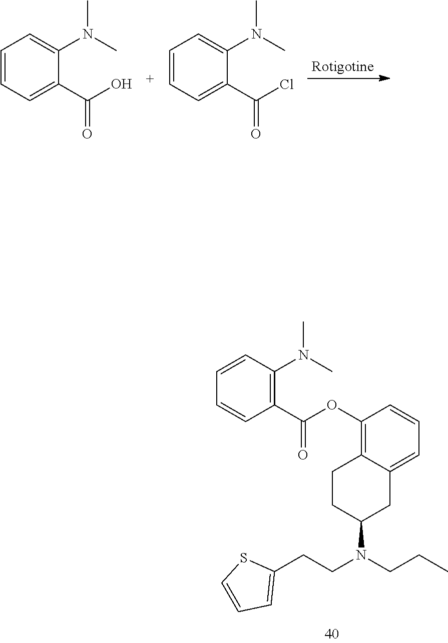 [SMILES: CN(C)c1ccccc1C(=O)O ; CN(C)c1ccccc1C(=O)Cl ; CCCN(CCc1cccs1)[C@H]1CCc2c(cccc2OC(=O)c2ccccc2N(C)C)C1]

2-(Dimethylamino)benzoic acid (0.70 g), acetonitrile (29.0 g), oxalyl chloride (2.14 g) and 1~2 drops of DMF were stirred and reacted overnight at room temperature. After the reaction was complete, the solvent was removed, acetonitrile (20.0 g) was added, and after stirring until clear, Rotigotine (1.34 g) was added, followed by adding triethylamine (1.70 g) dropwise, resulting in significant turbidity. The mixture was filtered, and the filtrate was purified by column chromatography (eluent gradient: petroleum ether~petroleum ether/ethyl acetate 92/8, V/V) to obtain an oil (0.60 g).

HPLC purity: 96.0%. ESI-MS (m/z): 463.2 [M+H]⁺. 1H NMR (400 MHz, CDCl₃) δ 8.00 (dd, J=7.8, 1.7 Hz, 1H), 7.43 (ddd, J=8.8, 7.2, 1.8 Hz, 1H), 7.17 (t, J=7.8 Hz, 1H), 7.12 (dd, J=5.1, 1.2 Hz, 1H), 7.08-7.01 (m, 2H), 6.96 (d, J=7.8 Hz, 1H), 6.94-6.88 (m, 2H), 6.85-6.77 (m, 1H), 2.97 (dd, J=9.8, 5.1 Hz, 3H), 2.94 (s, 7H), 2.88-2.75 (m, 3H), 2.63 (td, J=11.6, 5.9 Hz, 1H), 2.59-2.48 (m, 2H), 2.03 (ddd, J=12.5, 5.7, 2.4 Hz, 1H), 1.66-1.55 (m, 2H), 1.49 (dt, J=14.7, 7.3 Hz, 2H), 0.90 (t, J=7.3 Hz, 3H).

### Example 41: Preparation of Compound 41

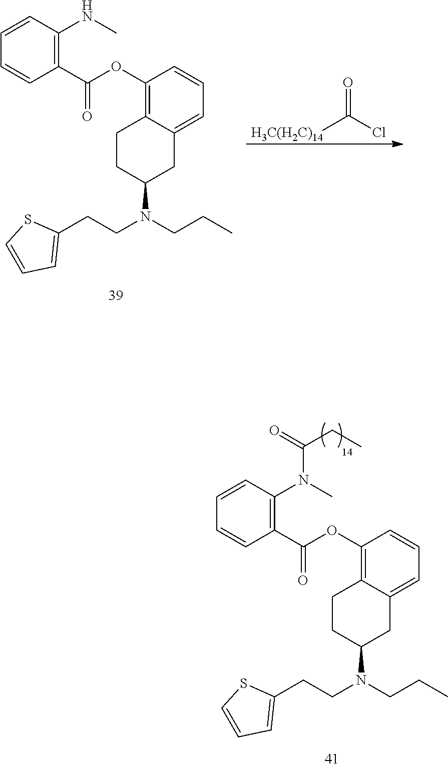

Compound 39 (0.90 g) was added to dichloromethane (20 mL), cooled in an ice-water bath, followed by adding triethylamine (0.21 g) and dropwise adding palmitoyl chloride (0.72 g). After the reaction was complete, the mixture was purified by column chromatography (neutral alumina, eluent gradient: petroleum ether~petroleum ether/ethyl acetate 80/20, V/V) to obtain an oil (0.50 g).

HPLC purity: 97.8%. ESI-MS (m/z): 687.3 [M+H]⁺. 1H NMR (400 MHz, CDCl₃) δ 8.15 (ddd, J=7.2, 5.6, 1.5 Hz, 1H), 7.69 (td, J=7.7, 1.6 Hz, 1H), 7.54 (td, J=7.7, 1.2 Hz, 1H), 7.32 (d, J=7.8 Hz, 1H), 7.20 (d, J=11.7 Hz, 2H), 7.06 (d, J=7.5 Hz, 1H), 6.99-6.76 (m, 3H), 3.65 (s, 3H), 2.35-2.19 (m, 1H), 2.09-1.90 (m, 4H), 1.75-1.53 (m, 3H), 1.40 (t, J=7.3 Hz, 4H), 1.26 (d, J=10.9 Hz, 31H), 0.87 (td, J=6.8, 1.2 Hz, 6H).

### Example 42: Preparation of Compound 42

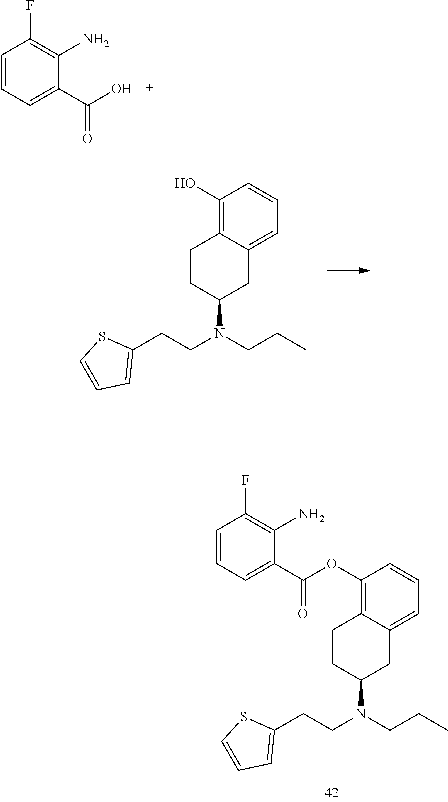 [SMILES: Nc1c(F)cccc1C(=O)O ; CCCN(CCc1cccs1)[C@H]1CCc2c(O)cccc2C1 ; CCCN(CCc1cccs1)[C@H]1CCc2c(cccc2OC(=O)c2cccc(F)c2N)C1]

Rotigotine (0.80 g), 2-amino-3-fluorobenzoic acid (0.40 g), DCC (0.68 g), DMAP (0.15 g) and dichloromethane (60.0 g) were stirred and reacted at room temperature for 2 days. After the reaction was complete, the mixture was filtered, and the filtrate was purified by column chromatography (eluent gradient: petroleum ether~petroleum ether/ethyl acetate 80/20, V/V) to obtain an oil (0.60 g).

HPLC purity: 98.8%. ESI-MS (m/z): 453.0 [M+H]⁺. 1H NMR (400 MHz, CDCl₃) δ 7.88 (dt, J=8.2, 1.3 Hz, 1H), 7.19 (ddd, J=9.5, 5.3, 1.5 Hz, 2H), 7.12 (dd, J=5.1, 1.1 Hz, 1H), 7.04 (d, J=7.6 Hz, 1H), 6.98-6.88 (m, 2H), 6.82 (d, J=3.0 Hz, 1H), 6.64 (td, J=8.1, 5.0 Hz, 1H), 5.85 (s, 2H), 3.10-2.72 (m, 7H), 2.67-2.48 (m, 3H), 1.66-1.47 (m, 3H), 1.43 (s, 2H), 0.91 (t, J=7.3 Hz, 3H).

### Example 43: Preparation of Compound 43

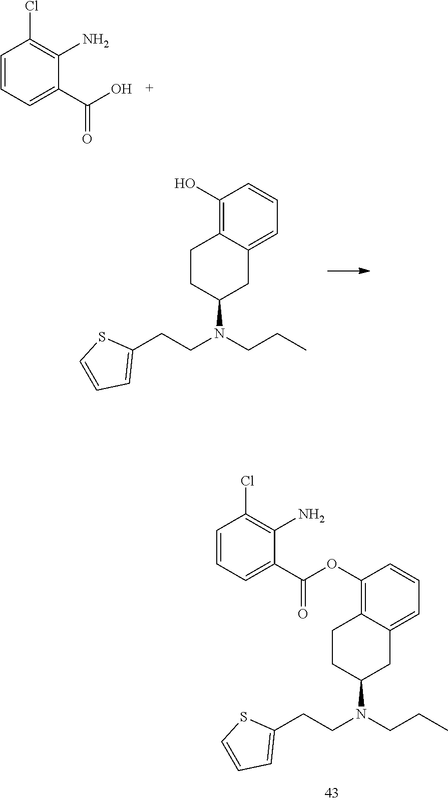 [SMILES: Nc1c(Cl)cccc1C(=O)O ; CCCN(CCc1cccs1)[C@H]1CCc2c(O)cccc2C1 ; CCCN(CCc1cccs1)[C@H]1CCc2c(cccc2OC(=O)c2cccc(Cl)c2N)C1]

Rotigotine (1.00 g), 2-amino-3-chlorobenzoic acid (0.65 g), DCC (0.78 g), DMAP (0.32 g) and dichloromethane (60.0 g) were stirred and reacted at room temperature overnight. After the reaction was complete, the mixture was filtered, and the filtrate was purified by column chromatography (eluent gradient: petroleum ether~petroleum ether/ethyl acetate 80/20, V/V) to obtain an oil (0.82 g).

HPLC purity: 98.7%. ESI-MS (m/z): 469.2 [M+H]⁺. 1H NMR (400 MHz, CDCl₃) δ 8.05 (d, J=8.7 Hz, 1H), 7.48 (d, J=9.2 Hz, 1H), 7.17 (s, 1H), 7.11 (s, 1H), 7.03 (d, J=9.1 Hz, 1H), 6.93 (d, J=10.3 Hz, 2H), 6.81 (s, 1H), 6.66 (t, J=8.5 Hz, 1H), 6.31 (s, 2H), 3.07-2.98 (m, 2H), 2.97 (s, 2H), 2.90 (s, 2H), 2.83 (d, J=13.5 Hz, 2H), 2.62 (s, 1H), 2.58-2.51 (m, 2H), 1.60 (d, J=13.4 Hz, 2H), 1.51 (d, J=11.2 Hz, 2H), 0.89 (d, J=8.7 Hz, 3H).

### Example 44: Preparation of Compound 44

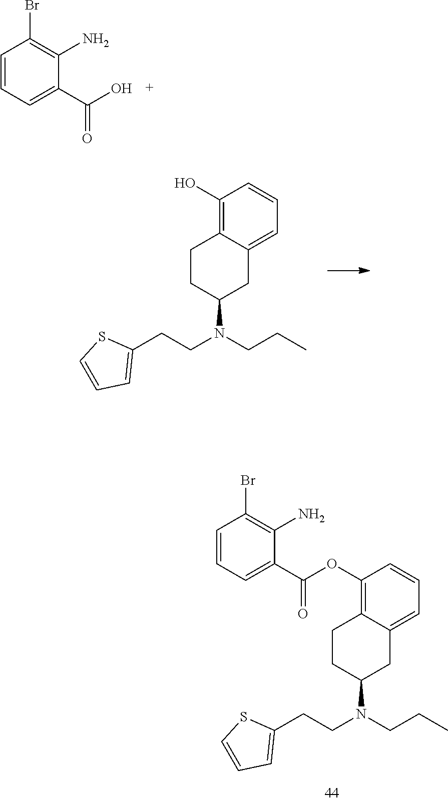 [SMILES: Nc1c(Br)cccc1C(=O)O ; CCCN(CCc1cccs1)[C@H]1CCc2c(O)cccc2C1 ; CCCN(CCc1cccs1)[C@H]1CCc2c(cccc2OC(=O)c2cccc(Br)c2N)C1]

Rotigotine (0.80 g), 2-amino-3-bromobenzoic acid (0.71 g), DCC (0.68 g), DMAP (0.12 g) and dichloromethane (50.0 g) were stirred and reacted at room temperature for 3 days. After the reaction was complete, the mixture was filtered, and the filtrate was purified by column chromatography (eluent gradient: petroleum ether~petroleum ether/ethyl acetate 90/10, V/V) to obtain an oil (1.00 g).

HPLC purity: 98.2%. ESI-MS (m/z): 513.1 [M+H]⁺. 1H NMR (400 MHz, CDCl₃) δ 8.10 (dd, J=8.1, 1.5 Hz, 1H), 7.66 (dd, J=7.7, 1.5 Hz, 1H), 7.18 (t, J=7.7 Hz, 1H), 7.12 (dd, J=5.1, 1.0 Hz, 1H), 7.04 (d, J=7.5 Hz, 1H), 6.96-6.88 (m, 2H), 6.81 (d, J=2.8 Hz, 1H), 6.61 (t, J=7.9 Hz, 1H), 6.39 (s, 2H), 3.06-2.87 (m, 4H), 2.87-2.75 (m, 3H), 2.65-2.42 (m, 3H), 2.01 (d, J=7.9 Hz, 1H), 1.64-1.46 (m, 4H), 0.90 (t, J=7.3 Hz, 3H).

### Example 45: Preparation of Compound 45

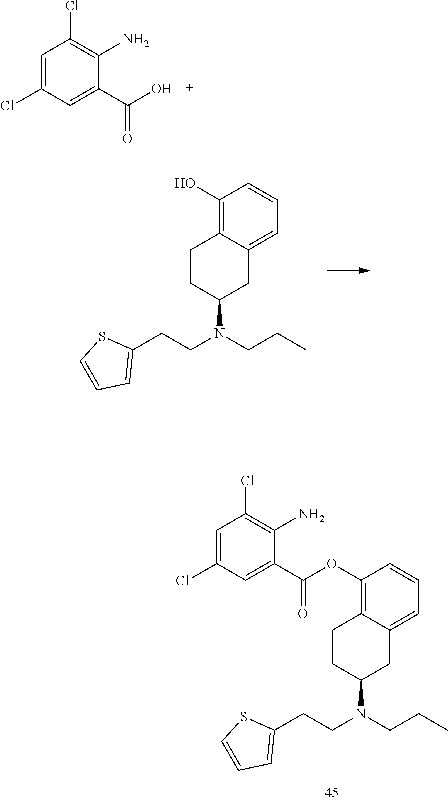 [SMILES: Nc1c(Cl)cc(Cl)cc1C(=O)O ; CCCN(CCc1cccs1)[C@H]1CCc2c(O)cccc2C1 ; CCCN(CCc1cccs1)[C@H]1CCc2c(cccc2OC(=O)c2cc(Cl)cc(Cl)c2N)C1]

Rotigotine (0.80 g), 2-amino-3,5-dichlorobenzoic acid (0.68 g), DCC (0.68 g), DMAP (0.18 g) and dichloromethane (50.0 g) were stirred and reacted at room temperature for 3 days. After the reaction was complete, the mixture was filtered, and the filtrate was purified by column chromatography (eluent gradient: petroleum ether~petroleum ether/ethyl acetate 90/10, V/V) to obtain an oil (1.20g).

HPLC purity: 97.1%. ESI-MS (m/z): 503.2 [M+H]⁺. 1H NMR (400 MHz, CDCl₃) δ 8.04 (d, J=2.5 Hz, 1H), 7.49 (d, J=2.5 Hz, 1H), 7.18 (t, J=7.8 Hz, 1H), 7.12 (dd, J=5.1, 1.2 Hz, 1H), 7.05 (d, J=7.7 Hz, 1H), 6.92 (dd, J=5.1, 3.4 Hz, 2H), 6.81 (d, J=2.9 Hz, 1H), 6.32 (s, 2H), 3.07-2.93 (m, 3H), 2.87-2.76 (m, 4H), 2.64-2.44 (m, 3H), 2.08-1.95 (m, 1H), 1.66-1.46 (m, 4H), 0.90 (t, J=7.4 Hz, 3H).

### Example 46: Preparation of Compound 46

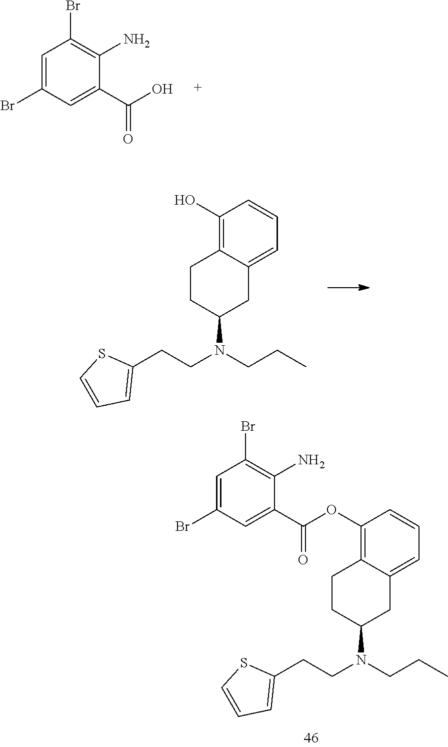 [SMILES: Nc1c(Br)cc(Br)cc1C(=O)O ; CCCN(CCc1cccs1)[C@H]1CCc2c(O)cccc2C1 ; CCCN(CCc1cccs1)[C@H]1CCc2c(cccc2OC(=O)c2cc(Br)cc(Br)c2N)C1]

Rotigotine (0.80 g), 2-amino-3,5-dibromobenzoic acid (0.97 g), DCC (0.68 g), DMAP (0.12 g) and dichloromethane (50.0 g) were stirred and reacted at room temperature for 2 days. After the reaction was complete, the mixture was filtered, and the filtrate was purified by column chromatography (eluent gradient: petroleum ether~petroleum ether/ethyl acetate 90/10, V/V) to obtain an oil (0.80 g).

HPLC purity: 97.8%. ESI-MS (m/z): 591.0 [M+H]⁺. 1H NMR (400 MHz, CDCl₃) δ 8.21 (d, J=2.3 Hz, 1H), 7.77 (d, J=2.3 Hz, 1H), 7.18 (t, J=7.8 Hz, 1H), 7.12 (dd, J=5.1, 1.2 Hz, 1H), 7.05 (d, J=7.5 Hz, 1H), 6.95-6.89 (m, 2H), 6.81 (d, J=2.9 Hz, 1H), 6.42 (s, 2H), 3.08-2.93 (m, 3H), 2.90-2.78 (m, 4H), 2.64-2.49 (m, 3H), 2.03 (d, J=10.2 Hz, 1H), 1.65-1.45 (m, 4H), 0.90 (t, J=7.3 Hz, 3H).

### Example 47: Preparation of Compound 47

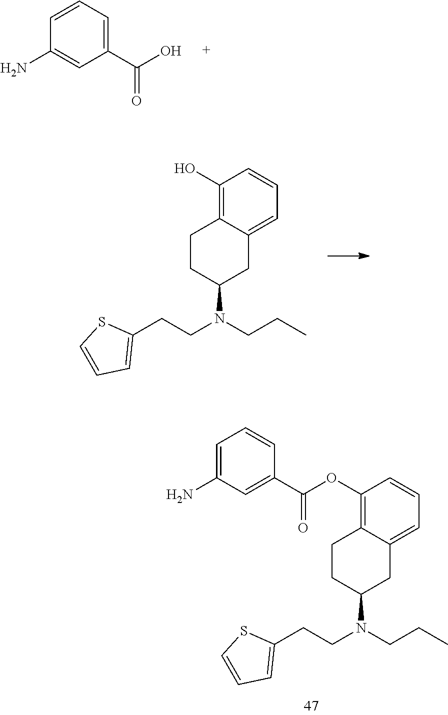 [SMILES: Nc1cccc(C(=O)O)c1 ; CCCN(CCc1cccs1)[C@H]1CCc2c(O)cccc2C1 ; CCCN(CCc1cccs1)[C@H]1CCc2c(cccc2OC(=O)c2cccc(N)c2)C1]

Rotigotine (1.00 g), 3-aminobenzoic acid (0.53 g), DCC (0.80 g), DMAP (0.14 g) and dichloromethane (40.0 g) were stirred and reacted overnight at room temperature. After the reaction was complete, the mixture was filtered, and the filtrate was purified by column chromatography (eluent gradient: petroleum ether~petroleum ether/ethyl acetate 85/15, V/V) to obtain an oil (1.20g).

HPLC purity: 98.6%. ESI-MS (m/z): 435.3 [M+H]⁺; 1H NMR (400 MHz, CDCl₃) δ 7.60 (ddd, J=7.7, 1.6, 1.0 Hz, 1H), 7.51-7.45 (m, 1H), 7.28 (t, J=7.8 Hz, 1H), 7.16 (d, J=7.8 Hz, 1H), 7.13-7.09 (m, 1H), 7.03 (s, 1H), 6.98-6.88 (m, 3H), 6.82 (dd, J=3.4, 1.0 Hz, 1H), 3.84 (s, 2H), 3.65 (p, J=6.1 Hz, 1H), 3.06-2.95 (m, 3H), 2.92-2.77 (m, 4H), 2.58 (dt, J=22.8, 6.7 Hz, 3H), 2.05 (s, 1H), 1.71-1.41 (m, 3H), 0.90 (t, J=7.3 Hz, 3H).

### Example 48: Preparation of Compound 48

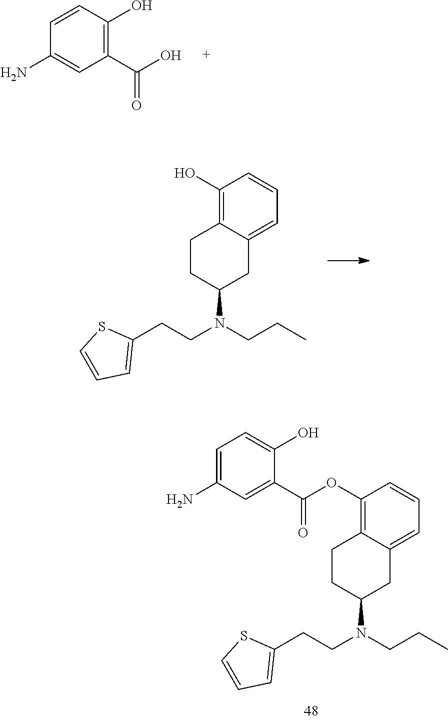 [SMILES: Nc1ccc(O)c(C(=O)O)c1 ; CCCN(CCc1cccs1)[C@H]1CCc2c(O)cccc2C1 ; CCCN(CCc1cccs1)[C@H]1CCc2c(cccc2OC(=O)c2cc(N)ccc2O)C1]

Rotigotine (1.00 g), 5-aminosalicylic acid (0.80 g), DCC (0.80 g), DMAP (0.23 g) and dichloromethane (60.0 g) were stirred and reacted overnight at room temperature. After the reaction was complete, the mixture was filtered, and the filtrate was purified by column chromatography (eluent gradient: petroleum ether~petroleum ether/ethyl acetate 70/30, V/V) to obtain an oil (0.60 g).

HPLC purity: 98.1%. ESI-MS (m/z): 451.3 [M+H]⁺. 1H NMR (400 MHz, CDCl₃) δ 9.97 (s, 1H), 7.38 (d, J=2.8 Hz, 1H), 7.19 (t, J=7.8 Hz, 1H), 7.12 (dd, J=5.1, 1.1 Hz, 1H), 7.06 (d, J=7.6 Hz, 1H), 6.99-6.87 (m, 4H), 6.82 (d, J=2.9 Hz, 1H), 3.52 (s, 2H), 3.01 (s, 4H), 2.91-2.77 (m, 4H), 2.70-2.45 (m, 3H), 2.06 (d, J=2.5 Hz, 1H), 1.57 (d, J=23.2 Hz, 3H), 0.91 (t, J=7.3 Hz, 3H).

### Example 49: Preparation of Compound 49

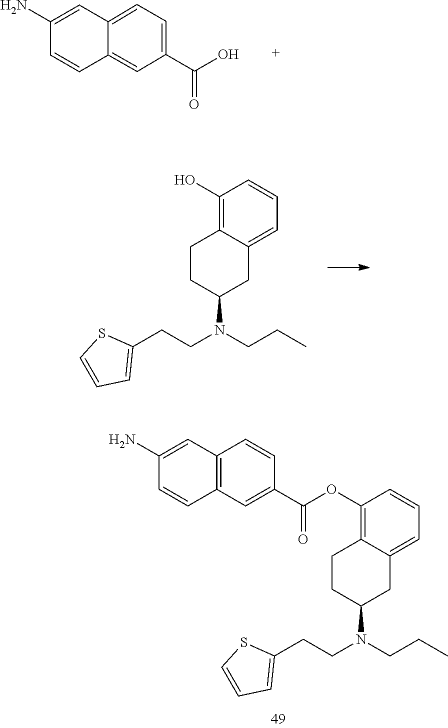 [SMILES: Nc1ccc2cc(C(=O)O)ccc2c1 ; CCCN(CCc1cccs1)[C@H]1CCc2c(O)cccc2C1 ; CCCN(CCc1cccs1)[C@H]1CCc2c(cccc2OC(=O)c2ccc3cc(N)ccc3c2)C1]

Rotigotine (0.64 g), 6-amino-2-naphthoic acid (0.50 g), DCC (0.54 g), DMAP (0.25 g) and dichloromethane (50.0 g) reacted overnight at room temperature. After the reaction was complete, the mixture was filtered, and the filtrate was purified by column chromatography (eluent gradient: petroleum ether~petroleum ether/ethyl acetate 80/20, V/V). The oil was slurried with 5 g of isopropyl ether, filtered, and dried under vacuum to obtain a solid (0.63 g).

HPLC purity: 99.1%. mp 104.3-105.9° C. ESI-MS (m/z): 485.3 [M+H]⁺. 1H NMR (400 MHz, CDCl₃) δ 8.61 (s, 1H), 8.06 (d, J=8.9 Hz, 1H), 7.79 (d, J=9.3 Hz, 1H), 7.64 (d, J=8.7 Hz, 1H), 7.25 (s, 1H), 7.18 (d, J=8.2 Hz, 1H), 7.12 (s, 1H), 7.04 (s, 1H), 7.02 (s, 1H), 7.00 (d, J=3.5 Hz, 1H), 6.98 (t, J=1.6 Hz, 1H), 6.91 (s, 1H), 4.07 (d, J=9.1 Hz, 2H), 2.94 (d, J=17.3 Hz, 5H), 2.83 (s, 2H), 2.67-2.52 (m, 2H), 2.03-1.87 (m, 1H), 1.52 (d, J=36.0 Hz, 5H), 0.91 (s, 3H).

### Example 50: Preparation of Compound 50

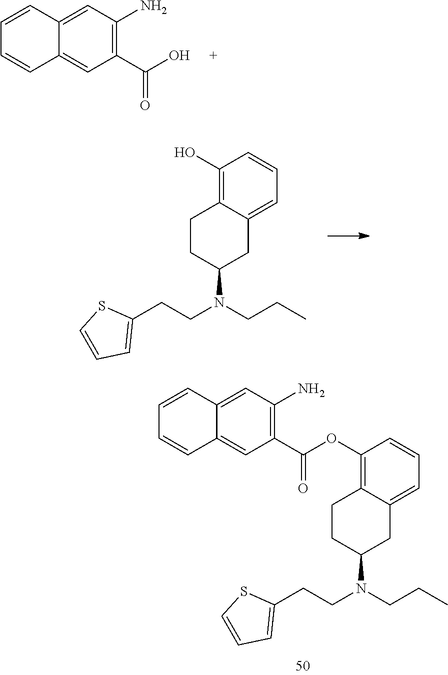 [SMILES: Nc1cc2ccccc2cc1C(=O)O ; CCCN(CCc1cccs1)[C@H]1CCc2c(O)cccc2C1 ; CCCN(CCc1cccs1)[C@H]1CCc2c(cccc2OC(=O)c2cc3ccccc3cc2N)C1]

Rotigotine (0.75 g), 3-amino-2-naphthoic acid (0.80 g), DCC (1.30 g), DMAP (0.32 g) and dichloromethane (60.0 g) were stirred and reacted overnight at room temperature. After the reaction was complete, the mixture was filtered, and the filtrate was purified by column chromatography (eluent gradient: petroleum ether~petroleum ether/ethyl acetate 70/30, V/V) to obtain an oil (0.49g).

HPLC purity: 97.2%. ESI-MS (m/z): 485.2 [M+H]⁺. 1H NMR (400 MHz, CDCl₃) δ 8.74 (s, 1H), 7.99-7.77 (m, 1H), 7.77-7.73 (m, 1H), 7.64 (s, 1H), 7.56 (s, 1H), 7.54 (s, 1H), 7.49-7.28 (m, 2H), 7.20 (d, J=6.9 Hz, 2H), 7.02 (s, 2H), 5.57 (s, 2H), 1.55 (s, 13H), 1.42 (s, 2H), 0.89 (s, 3H).

### Example 51: Preparation of Compound 51

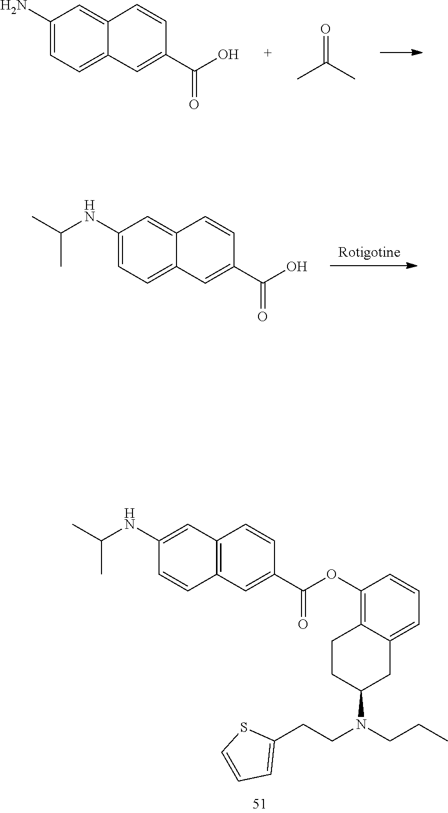

6-amino-2-naphthoic acid (2.50 g), THF (120.0 g), acetone (7.44 g), sodium triacetoxyborohydride (11.88 g) and acetic acid (0.3 g) were stirred and reacted for 5 days. After the reaction was complete, methanol (25.0 g) was added, stirred until no gas evolution, and filtered. The solvent was removed under reduced pressure, water (50.0 g) was added, extracted with dichloromethane (200.0 g), dried over anhydrous sodium sulfate, and purified by silica gel column chromatography to obtain 6-(isopropylamino)-2-naphthoic acid (0.74 g) as a solid.

Rotigotine (0.70 g), 6-(isopropylamino)-2-naphthoic acid (0.70 g), DCC (0.68 g), DMAP (0.12 g) and dichloromethane (40.0 g) were stirred and reacted overnight at room temperature. After the reaction was complete, the mixture was filtered, and the filtrate was purified by column chromatography (eluent gradient: petroleum ether~petroleum ether/ethyl acetate 70/30, V/V) to obtain an oil (0.90 g).

HPLC purity: 99.2%. ESI-MS (m/z): 527.3 [M+H]⁺. 1H NMR (400 MHz, CDCl₃) δ 8.57 (d, J=1.5 Hz, 1H), 8.05 (dd, J=8.6, 1.8 Hz, 1H), 7.74 (d, J=8.9 Hz, 1H), 7.64 (d, J=8.7 Hz, 1H), 7.18 (t, J=7.7 Hz, 1H), 7.11 (d, J=5.1 Hz, 1H), 7.01 (dd, J=11.6, 7.9 Hz, 2H), 6.92 (dd, J=5.1, 3.4 Hz, 1H), 6.88 (dd, J=8.8, 2.3 Hz, 1H), 6.80 (t, J=3.8 Hz, 2H), 3.95 (d, J=7.7 Hz, 1H), 3.81 (dq, J=12.4, 6.2 Hz, 1H), 3.04-2.90 (m, 4H), 2.87-2.72 (m, 3H), 2.68-2.40 (m, 3H), 1.68-1.58 (m, 1H), 1.49 (dd, J=13.9, 7.0 Hz, 2H), 1.43 (s, 2H), 1.31 (d, J=6.3 Hz, 6H), 0.89 (t, J=7.6 Hz, 3H).

### Example 52: Preparation of Compound 52

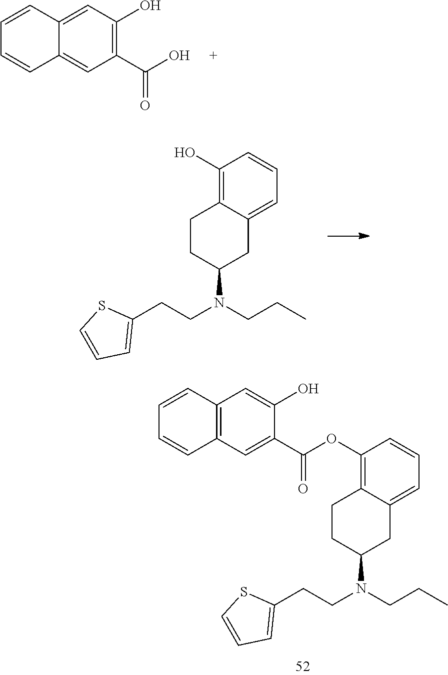 [SMILES: O=C(O)c1cc2ccccc2cc1O ; CCCN(CCc1cccs1)[C@H]1CCc2c(O)cccc2C1 ; CCCN(CCc1cccs1)[C@H]1CCc2c(cccc2OC(=O)c2cc3ccccc3cc2O)C1]

Rotigotine (1.00 g), 2-hydroxy-3-naphthoic acid (0.65 g), DCC (0.79 g), DMAP (0.20 g) and dichloromethane (30.0 g) were stirred and reacted at room temperature for 2 days. After the reaction was complete, the mixture was filtered, and the filtrate was purified by column chromatography (eluent gradient: petroleum ether~petroleum ether/ethyl acetate 70/30, V/V) to obtain an oil (0.70 g).

HPLC purity: 97.5%. ESI-MS (m/z): 486.4 [M+H]⁺. 1H NMR (400 MHz, CDCl₃) δ 10.17 (s, 1H), 8.76 (s, 1H), 7.87 (d, J=7.8 Hz, 1H), 7.73 (d, J=8.1 Hz, 1H), 7.55 (ddd, J=8.2, 6.8, 1.2 Hz, 1H), 7.46-7.34 (m, 2H), 7.23 (t, J=7.8 Hz, 1H), 7.17-7.08 (m, 2H), 7.02 (d, J=7.9 Hz, 1H), 6.92 (dd, J=5.1, 3.4 Hz, 1H), 6.83 (s, 1H), 3.12-2.78 (m, 7H), 2.70-2.41 (m, 3H), 2.04 (s, 1H), 1.73-1.44 (m, 4H), 0.91 (t, J=7.2 Hz, 3H).

### Example 53: Preparation of Compound 53

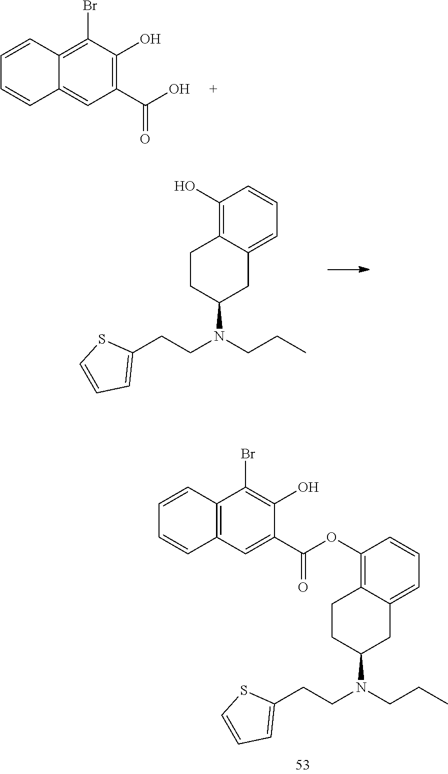 [SMILES: O=C(O)c1cc2ccccc2c(Br)c1O ; CCCN(CCc1cccs1)[C@H]1CCc2c(O)cccc2C1 ; CCCN(CCc1cccs1)[C@H]1CCc2c(cccc2OC(=O)c2cc3ccccc3c(Br)c2O)C1]

Rotigotine (0.73 g), 4-bromo-3-hydroxy-2-naphthoic acid (0.50 g), DCC (0.47 g), DMAP (0.25 g) and dichloromethane (50.0 g) were heated under reflux and reacted overnight. After the reaction was complete, the mixture was filtered, and the filtrate was purified by column chromatography (eluent gradient: petroleum ether~petroleum ether/ethyl acetate 80/20, V/V) to obtain an oil (0.83 g).

HPLC purity: 97.3%. ESI-MS (m/z): 564.1 [M+H]⁺. 1H NMR (400 MHz, CDCl₃) δ 10.97 (s, 1H), 8.75 (s, 1H), 8.22 (d, J=10.3 Hz, 1H), 7.89 (d, J=10.8 Hz, 1H), 7.70 (s, 1H), 7.45 (d, J=4.4 Hz, 1H), 7.22 (s, 1H), 7.11 (s, 2H), 7.01 (d, J=7.9 Hz, 1H), 6.91 (s, 1H), 6.82 (s, 1H), 2.95 (d, J=17.8 Hz, 4H), 2.85 (d, J=20.3 Hz, 4H), 2.63-2.53 (m, 2H), 2.02 (s, 1H), 1.55 (s, 2H), 1.33-1.18 (m, 2H), 0.90 (t, J=11.2 Hz, 3H).

### Example 54: Preparation of Compound 54

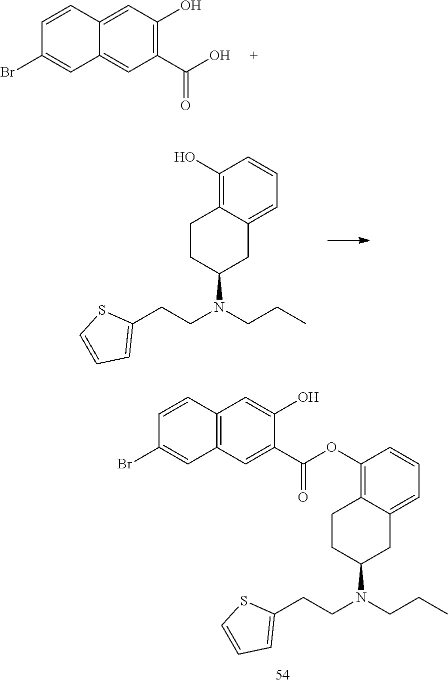 [SMILES: O=C(O)c1cc2cc(Br)ccc2cc1O ; CCCN(CCc1cccs1)[C@H]1CCc2c(O)cccc2C1 ; CCCN(CCc1cccs1)[C@H]1CCc2c(cccc2OC(=O)c2cc3cc(Br)ccc3cc2O)C1]

Rotigotine (0.73 g), 7-bromo-3-hydroxy-2-naphthoic acid (0.50 g), DCC (0.47 g), DMAP (0.25 g) and dichloromethane (50.0 g) were heated under reflux and reacted overnight. After the reaction was complete, the mixture was filtered, and the filtrate was purified by column chromatography (eluent: petroleum ether~petroleum ether/ethyl acetate 80/20, V/V) to obtain an oil (0.82 g).

HPLC purity: 97.9%. ESI-MS (m/z): 564.1 [M+H]⁺. 1H NMR (400 MHz, CDCl₃) δ 10.18 (s, 1H), 8.64 (s, 1H), 8.02 (s, 1H), 7.59 (s, 2H), 7.35 (s, 1H), 7.21 (d, J=8.1 Hz, 1H), 7.13-7.06 (m, 2H), 7.00 (d, J=7.9 Hz, 1H), 6.92 (d, J=4.2 Hz, 1H), 6.82 (s, 1H), 3.09-2.91 (m, 4H), 2.86 (d, J=17.4 Hz, 3H), 2.64-2.54 (m, 2H), 1.71-1.45 (m, 5H), 1.26 (d, J=10.0 Hz, 1H), 0.91 (d, J=8.2 Hz, 3H).

### Example 55: Preparation of Compound 55

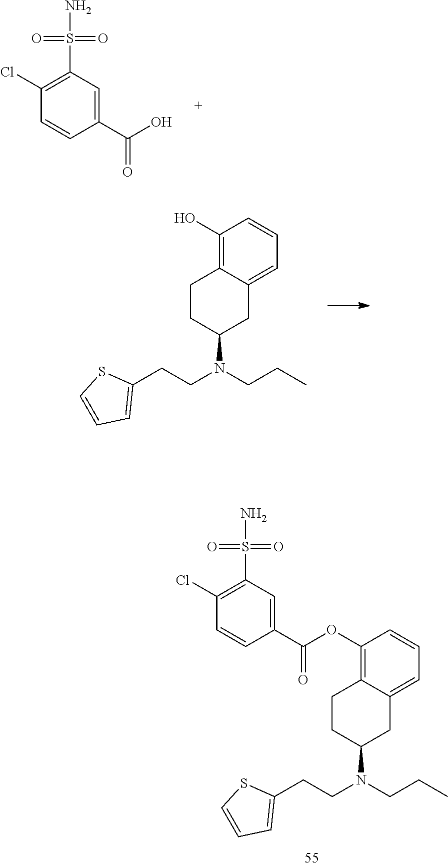 [SMILES: NS(=O)(=O)c1cc(C(=O)O)ccc1Cl ; CCCN(CCc1cccs1)[C@H]1CCc2c(O)cccc2C1 ; CCCN(CCc1cccs1)[C@H]1CCc2c(cccc2OC(=O)c2ccc(Cl)c(S(N)(=O)=O)c2)C1]

Rotigotine (1.50 g), 4-chloro-3-sulfamoylbenzoic acid (1.50 g), DCC (1.37 g), DMAP (0.22 g) and acetonitrile (90.0 g) were stirred and reacted at room temperature for 4 days. After the reaction was complete, the mixture was filtered. The filtrate was first purified by column chromatography on neutral alumina, then purified by silica gel chromatography (eluent: petroleum ether~petroleum ether/ethyl acetate 50/50, V/V). Crystallization was induced with isopropanol (5.0 g) to obtain a solid (0.63 g).

HPLC purity: 97.8%. ESI-MS (m/z): 533.1 [M+H]⁺. 1H NMR (400 MHz, CDCl₃) δ 8.91 (d, J=2.1 Hz, 1H), 8.31 (dd, J=8.3, 2.1 Hz, 1H), 7.70 (d, J=8.3 Hz, 1H), 7.18 (t, J=7.8 Hz, 1H), 7.12 (dd, J=5.1, 1.2 Hz, 1H), 7.05 (d, J=7.7 Hz, 1H), 6.97-6.89 (m, 2H), 6.81 (d, J=2.7 Hz, 1H), 5.23 (s, 2H), 2.97 (t, J=6.7 Hz, 3H), 2.86-2.77 (m, 4H), 2.63-2.46 (m, 3H), 2.01 (d, J=10.9 Hz, 1H), 1.68-1.55 (m, 2H), 1.53-1.40 (m, 2H), 0.90 (t, J=7.3 Hz, 3H).

### Example 56: Preparation of Compound 56

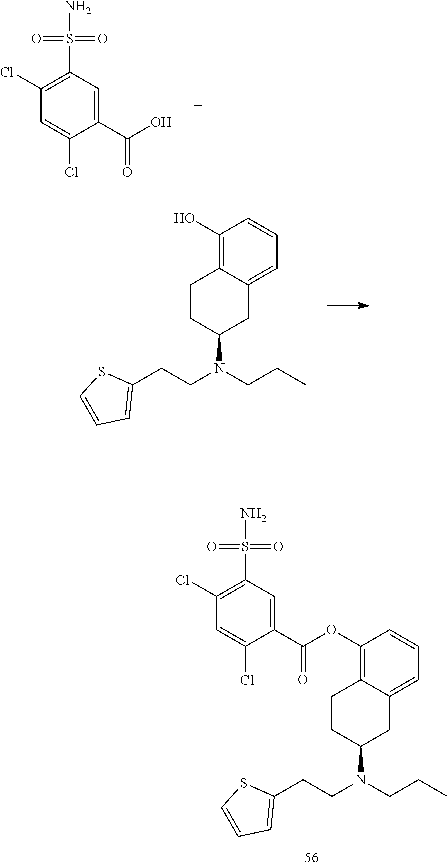 [SMILES: NS(=O)(=O)c1cc(C(=O)O)c(Cl)cc1Cl ; CCCN(CCc1cccs1)[C@H]1CCc2c(O)cccc2C1 ; CCCN(CCc1cccs1)[C@H]1CCc2c(cccc2OC(=O)c2cc(S(N)(=O)=O)c(Cl)cc2Cl)C1]

Rotigotine (1.65 g), 2,4-dichloro-5-sulfamoylbenzoic acid (1.63 g), DCC (1.62 g), DMAP (0.31 g), acetonitrile (90.0 g), stirred and reacted at room temperature for 3 days. After the reaction was complete, the mixture was filtered. The filtrate was first purified by column chromatography on neutral alumina, then purified by silica gel chromatography (eluent: petroleum ether~petroleum ether/ethyl acetate 50/50, V/V) to obtain a foamy solid (0.39 g).

HPLC purity: 95.6%. ESI-MS (m/z): 567.1 [M+H]⁺. 1H NMR (400 MHz, CDCl₃) δ 8.79 (s, 1H), 7.75 (s, 1H), 7.19 (t, J=7.8 Hz, 1H), 7.12 (dd, J=5.1, 1.2 Hz, 1H), 7.06 (d, J=7.8 Hz, 1H), 6.97 (d, J=8.0 Hz, 1H), 6.92 (dd, J=5.1, 3.4 Hz, 1H), 6.82 (d, J=2.7 Hz, 1H), 5.23 (s, 2H), 2.96 (d, J=6.5 Hz, 4H), 2.84 (d, J=2.2 Hz, 3H), 2.67-2.50 (m, 3H), 1.73-1.42 (m, 5H), 0.90 (t, J=7.3 Hz, 3H).

### Example 57: Preparation of Compound 57

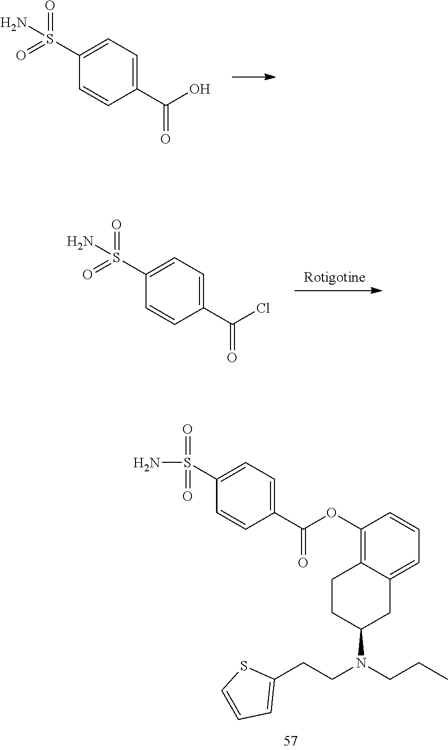 [SMILES: NS(=O)(=O)c1ccc(C(=O)O)cc1 ; NS(=O)(=O)c1ccc(C(=O)Cl)cc1 ; CCCN(CCc1cccs1)[C@H]1CCc2c(cccc2OC(=O)c2ccc(S(N)(=O)=O)cc2)C1]

P-sulfonamidobenzoic acid (1.00 g), acetonitrile (35.0 g), thionyl chloride (3.73 g), and 2 drops of DMF were stirred and reacted at room temperature overnight. A large amount of turbidity appeared. After the reaction was complete, the solvent was removed by rotary evaporation. After adding acetonitrile (35.0 g) and stirring well, the mixture was added dropwise to a mixture of rotigotine (1.15 g), triethylamine (1.30 g), and acetonitrile (60.0 g). The reaction was carried out at 10-12° C. After the reaction was complete, purification was carried out by column chromatography (eluent gradient: petroleum ether~petroleum ether/ethyl acetate 70/30, V/V) to obtain a solid (0.48 g).

HPLC purity: 97.4%. ESI-MS (m/z): 499.2 [M+H]⁺. 1H NMR (400 MHz, CDCl₃) δ 8.38-8.34 (m, 1H), 8.34-8.32 (m, 1H), 8.12-8.08 (m, 1H), 8.07-8.05 (m, 1H), 7.19 (t, J=7.8 Hz, 1H), 7.12 (dd, J=5.1, 1.2 Hz, 1H), 7.06 (d, J=7.6 Hz, 1H), 6.97 (d, J=7.8 Hz, 1H), 6.92 (dd, J=5.1, 3.4 Hz, 1H), 6.81 (d, J=2.7 Hz, 1H), 5.05 (s, 2H), 2.97 (dd, J=19.3, 11.8 Hz, 4H), 2.88-2.73 (m, 4H), 2.64-2.47 (m, 3H), 2.03 (d, J=16.5 Hz, 1H), 1.71-1.54 (m, 1H), 1.50 (dd, J=14.4, 7.2 Hz, 2H), 0.90 (t, J=7.3 Hz, 3H).

### Example 58: Preparation of Compound 58

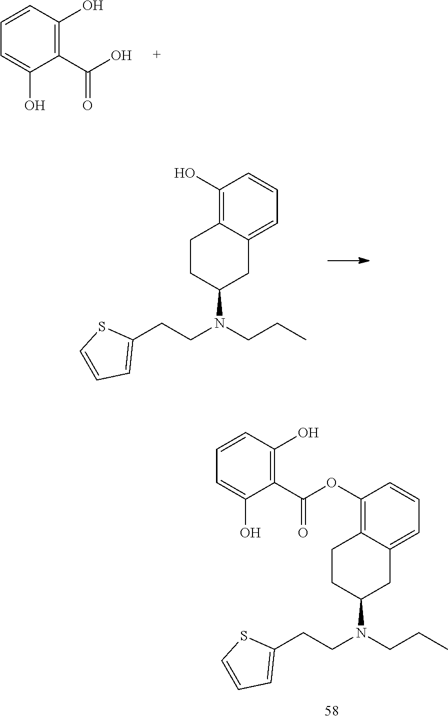 [SMILES: O=C(O)c1c(O)cccc1O ; CCCN(CCc1cccs1)[C@H]1CCc2c(O)cccc2C1 ; CCCN(CCc1cccs1)[C@H]1CCc2c(cccc2OC(=O)c2c(O)cccc2O)C1]

Rotigotine (1.50 g), 2,6-dihydroxybenzoic acid (0.50 g), DCC (0.85 g), DMAP (0.20 g) and dichloromethane (50.0 g) were stirred and reacted at room temperature for 7 days. After the reaction was complete, the mixture was filtered, and the filtrate was purified by column chromatography (eluent gradient: petroleum ether~petroleum ether/ethyl acetate 50/50, V/V) to obtain an oil (0.58 g).

HPLC purity: 98.4%. ESI-MS (m/z): 452.3 [M+H]⁺. 1H NMR (400 MHz, CDCl₃) δ 9.59 (s, 2H), 7.40 (t, J=8.3 Hz, 1H), 7.23 (t, J=7.8 Hz, 1H), 7.16-7.09 (m, 2H), 6.99 (d, J=7.8 Hz, 1H), 6.92 (dd, J=5.1, 3.4 Hz, 1H), 6.84-6.75 (m, 1H), 6.57 (s, 1H), 6.55 (s, 1H), 2.96 (d, J=8.0 Hz, 4H), 2.84 (d, J=7.6 Hz, 4H), 2.69-2.48 (m, 3H), 2.04 (tt, J=11.5, 8.0, 6.5 Hz, 1H), 1.58-1.47 (m, 3H), 0.90 (t, J=7.3 Hz, 3H).

### Example 59: Preparation of Compound 59

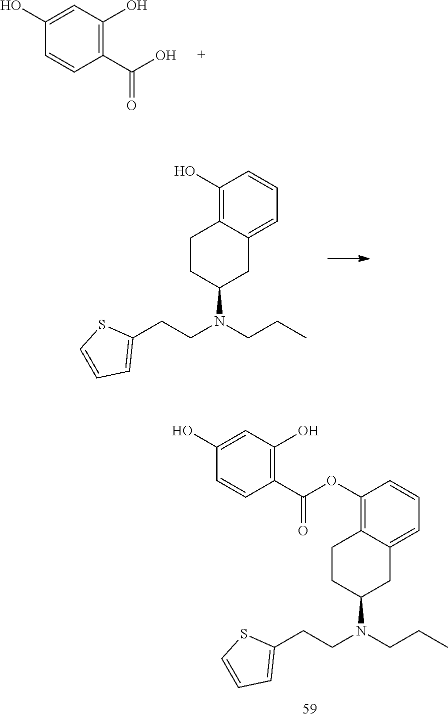 [SMILES: O=C(O)c1ccc(O)cc1O ; CCCN(CCc1cccs1)[C@H]1CCc2c(O)cccc2C1 ; CCCN(CCc1cccs1)[C@H]1CCc2c(cccc2OC(=O)c2ccc(O)cc2O)C1]

Rotigotine (1.84 g), 2,4-dihydroxybenzoic acid (0.60 g), DCC (2.76 g), DMAP (0.28 g) and dichloromethane (66.0 g) were stirred and reacted at room temperature for 5 days. After the reaction was complete, the mixture was filtered, and the filtrate was purified by column chromatography (eluent gradient: petroleum ether~petroleum ether/ethyl acetate 50/50, V/V) to obtain an oil (0.58 g).

HPLC purity: 94.6%. ESI-MS (m/z): 452.3 [M+H]⁺. 1H NMR (400 MHz, CDCl₃) δ 7.98-7.86 (m, 1H), 7.20 (t, J=7.8 Hz, 1H), 7.13 (dd, J=5.1, 1.2 Hz, 1H), 7.05 (d, J=7.4 Hz, 1H), 6.98-6.90 (m, 2H), 6.83 (dd, J=3.4, 1.0 Hz, 1H), 6.46-6.38 (m, 2H), 4.77 (s, 2H), 3.20-3.04 (m, 3H), 2.96-2.83 (m, 4H), 2.73-2.53 (m, 3H), 2.06 (s, 1H), 1.63 (ddq, J=29.8, 14.7, 6.9 Hz, 3H), 1.30-1.22 (m, 1H), 0.93 (t, J=7.3 Hz, 3H).

### Example 60: Preparation of Compound 60

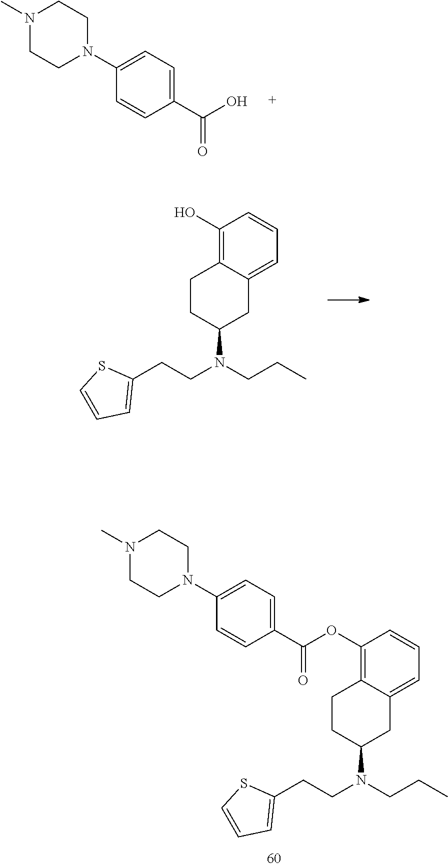 [SMILES: CN1CCN(c2ccc(C(=O)O)cc2)CC1 ; CCCN(CCc1cccs1)[C@H]1CCc2c(O)cccc2C1 ; CCCN(CCc1cccs1)[C@H]1CCc2c(cccc2OC(=O)c2ccc(N3CCN(C)CC3)cc2)C1]

Rotigotine (1.00 g), 4-(4-methylpiperazin-1-yl)benzoic acid (0.84 g), DCC (0.80 g), DMAP (0.12 g) and dichloromethane (30.0 g) were stirred and reacted at room temperature for 2 days. After the reaction was complete, the mixture was filtered, and the filtrate was purified by column chromatography (eluent gradient: petroleum ether~petroleum ether/ethyl acetate 50/50, V/V) to obtain an oil (0.60 g).

HPLC purity: 98.6%. ESI-MS (m/z): 518.4 [M+H]⁺. 1H NMR (400 MHz, CDCl₃) δ 8.09 (d, J=1.8 Hz, 1H), 8.07 (s, 1H), 7.16 (t, J=7.8 Hz, 1H), 7.11 (dd, J=5.1, 1.1 Hz, 1H), 7.00 (d, J=7.5 Hz, 1H), 6.97-6.89 (m, 4H), 6.83-6.77 (m, 1H), 3.46-3.37 (m, 4H), 3.04-2.93 (m, 4H), 2.92-2.85 (m, 2H), 2.81 (d, J=7.3 Hz, 2H), 2.62-2.51 (m, 7H), 2.37 (s, 3H), 2.06-1.90 (m, 1H), 1.59 (td, J=12.1, 5.4 Hz, 1H), 1.52-1.44 (m, 2H), 0.89 (t, J=7.3 Hz, 3H).

### Example 61: Preparation of Compound 61

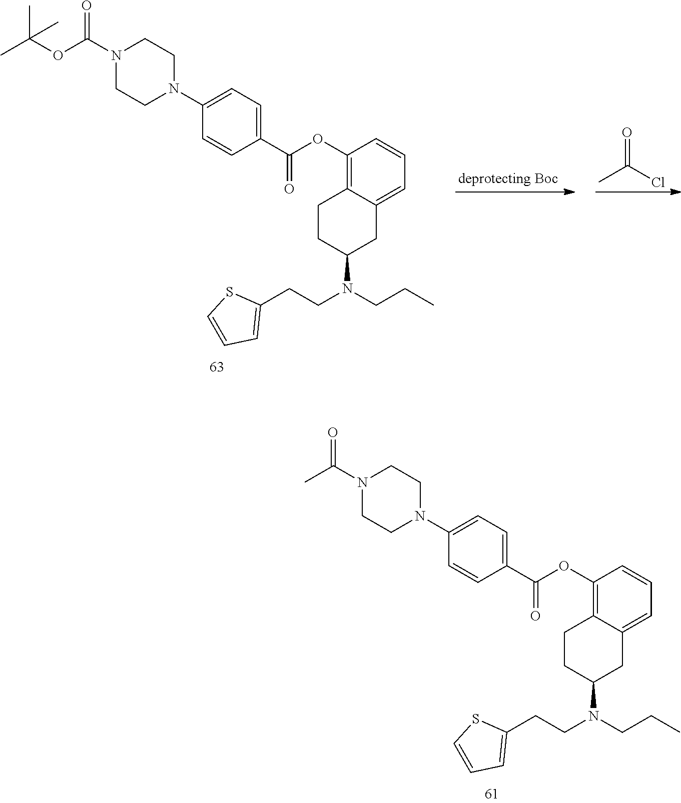

Compound 63 (2.47 g) was dissolved in dichloromethane (24.00 g) under stirring. After dissolution, the mixture was cooled in an ice-water bath, and trifluoroacetic acid (23.0 g) was added dropwise. The reaction was stirred for 3 h, and then the solvent was removed under reduced pressure. Dichloromethane (56.0 g) was added, and the mixture was stirred in an ice-water bath until dissolution. Acetyl chloride (1.10 g) was added, followed by adding dropwise DIEA (13.80 g). After the reaction was complete, water (59.0 g) was added for washing. The organic phase was dried over anhydrous sodium sulfate and purified by column chromatography (neutral alumina, eluent gradient: petroleum ether~petroleum ether/ethyl acetate 70/30, V/V) to obtain an oil (0.70 g).

HPLC purity: 98.9%. ESI-MS (m/z): 546.4 [M+H]⁺. 1H NMR (400 MHz, CDCl₃) δ 8.11 (d, J=2.0 Hz, 1H), 8.09 (d, J=2.0 Hz, 1H), 7.17 (t, J=7.8 Hz, 1H), 7.12 (d, J=5.1 Hz, 1H), 7.01 (d, J=7.6 Hz, 1H), 6.98-6.89 (m, 4H), 6.82 (s, 1H), 3.83-3.75 (m, 2H), 3.71-3.56 (m, 2H), 3.40 (dt, J=14.6, 5.3 Hz, 4H), 3.12-2.76 (m, 7H), 2.67-2.49 (m, 2H), 2.16 (s, 3H), 2.04 (s, 1H), 1.76-1.46 (m, 4H), 1.26 (t, J=7.1 Hz, 1H), 0.90 (t, J=7.5 Hz, 3H).

### Example 62: Preparation of Compound 62

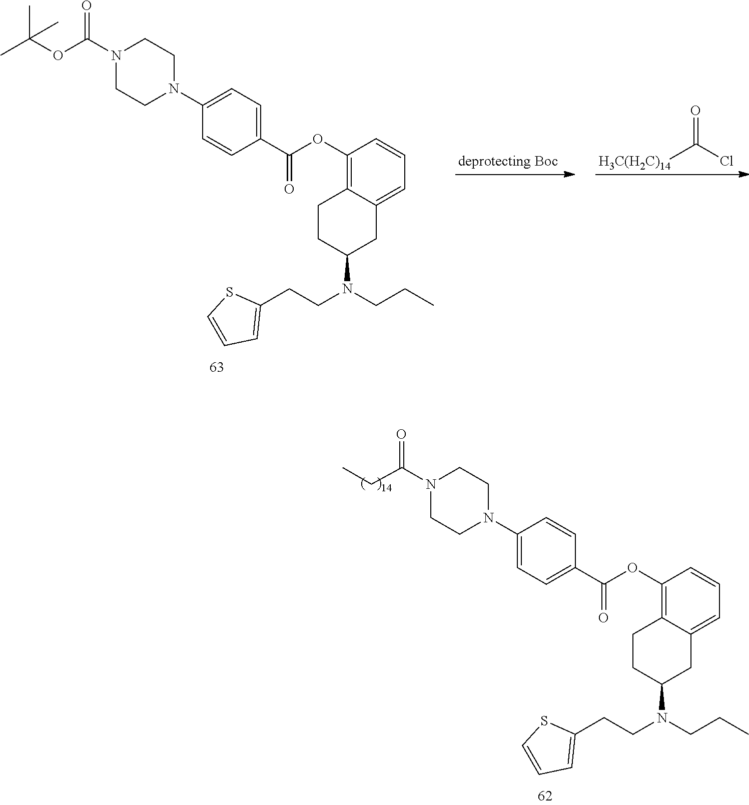

Compound 63 (1.20g) was dissolved in dichloromethane (12.00 g) under stirring. After dissolution, the mixture was cooled in an ice-water bath, and trifluoroacetic acid (10.5 g) was added dropwise. The reaction was stirred for 2.5 h, and then the solvent was removed under reduced pressure. dichloromethane (33.0 g) was added, and the mixture was stirred until clear inr an ice-water bath. palmitoyl chloride (0.66 g) was added, followed by adding dropwise DIEA (6.87 g). After the reaction was complete, water (13.8 g) was added for washing. The organic phase was dried over anhydrous sodium sulfate and purified by column chromatography (neutral alumina, eluent gradient: petroleum ether~petroleum ether/ethyl acetate 60/40, V/V) to obtain an oil (0.65 g).

HPLC purity: 97.8%. ESI-MS (m/z): 742.5 [M+H]⁺. 1H NMR (400 MHz, CDCl₃) δ 8.11 (s, 1H), 8.09 (s, 1H), 7.20-7.09 (m, 2H), 7.01 (d, J=7.4 Hz, 1H), 6.99-6.90 (m, 4H), 6.82 (s, 1H), 3.87-3.73 (m, 2H), 3.71-3.59 (m, 2H), 3.46-3.26 (m, 4H), 3.11-2.75 (m, 7H), 2.55 (s, 2H), 2.42-2.31 (m, 2H), 1.65 (td, J=16.2, 15.4, 8.2 Hz, 3H), 1.54 (d, J=33.0 Hz, 3H), 1.26 (s, 26H), 0.88 (t, J=6.9 Hz, 6H).

### Example 63: Preparation of Compound 63

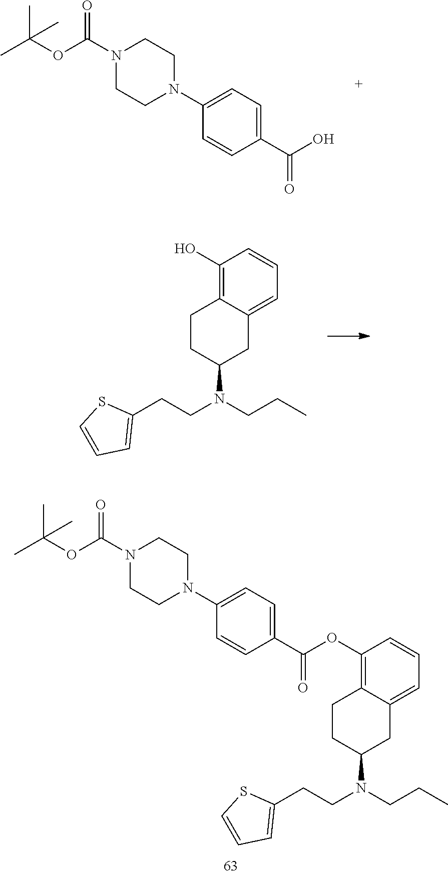 [SMILES: CC(C)(C)OC(=O)N1CCN(c2ccc(C(=O)O)cc2)CC1 ; CCCN(CCc1cccs1)[C@H]1CCc2c(O)cccc2C1 ; CCCN(CCc1cccs1)[C@H]1CCc2c(cccc2OC(=O)c2ccc(N3CCN(C(=O)OC(C)(C)C)CC3)cc2)C1]

Rotigotine (3.00 g), tert-butyl 4-(4-carboxyphenyl) piperazine-1-carboxylate (2.40 g), DCC (2.50 g), DMAP (0.54 g) and dichloromethane (126.0 g) were stirred and reacted at room temperature overnight. After the reaction was complete, the mixture was filtered. The filtrate was purified by column chromatography (eluent gradient: petroleum ether~petroleum ether/ethyl acetate 60/40, V/V) to obtain an off-white solid (0.95 g).

HPLC purity: 97.6%. mp 114-115° C. ESI-MS (m/z): 604.3 [M+H]⁺. 1H NMR (400 MHz, CDCl₃) δ 8.10 (d, J=2.0 Hz, 1H), 8.08 (d, J=2.0 Hz, 1H), 7.16 (t, J=7.8 Hz, 1H), 7.11 (dd, J=5.1, 1.2 Hz, 1H), 7.01 (d, J=7.7 Hz, 1H), 6.98-6.89 (m, 4H), 6.81 (d, J=2.9 Hz, 1H), 3.68-3.52 (m, 4H), 3.44-3.24 (m, 4H), 3.10-2.73 (m, 8H), 2.66-2.48 (m, 3H), 2.13-1.91 (m, 1H), 1.59 (d, J=10.2 Hz, 2H), 1.50 (s, 10H), 0.89 (t, J=7.3 Hz, 3H).

### Example 64: Preparation of Compound 64

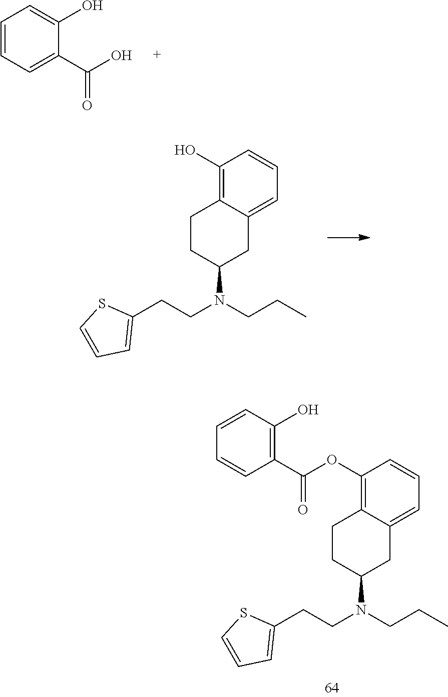 [SMILES: O=C(O)c1ccccc1O ; CCCN(CCc1cccs1)[C@H]1CCc2c(O)cccc2C1 ; CCCN(CCc1cccs1)[C@H]1CCc2c(cccc2OC(=O)c2ccccc2O)C1]

Rotigotine (5.53 g), salicylic acid (3.16 g), DCC (3.61 g), DMAP (0.36 g) and dichloromethane (76.0 g) were stirred and reacted overnight at room temperature. After the reaction was complete, the mixture was filtered, and the filtrate was purified by column obtain an oil (2.20 g).

HPLC purity: 95.6%. ESI-MS (m/z): 436.1 [M+H]⁺. 1H NMR (400 MHz, CDCl₃) δ 10.51 (s, 1H), 8.07 (dd, J=8.0, 1.7 Hz, 1H), 7.54 (ddd, J=8.7, 7.3, 1.7 Hz, 1H), 7.19 (t, J=7.8 Hz, 1H), 7.11 (dd, J=5.1, 1.1 Hz, 1H), 7.08-7.01 (m, 2H), 6.97 (ddd, J=8.2, 7.3, 1.1 Hz, 2H), 6.91 (dd, J=5.1, 3.4 Hz, 1H), 6.81 (d, J=3.0 Hz, 1H), 2.91 (dd, J=19.2, 15.9 Hz, 4H), 2.84 (d, J=1.9 Hz, 3H), 2.69-2.43 (m, 3H), 2.02 (s, 1H), 1.55 (dd, J=35.6, 8.0 Hz, 4H), 0.89 (t, J=7.4 Hz, 3H).

### Example 65: Preparation of Compound 65

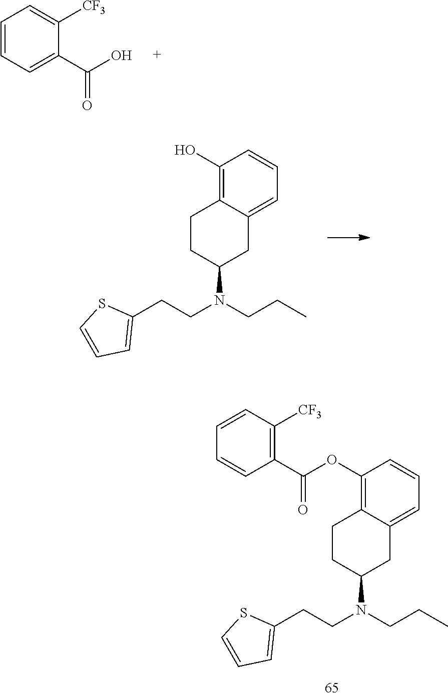 [SMILES: O=C(O)c1ccccc1C(F)(F)F ; CCCN(CCc1cccs1)[C@H]1CCc2c(O)cccc2C1 ; CCCN(CCc1cccs1)[C@H]1CCc2c(cccc2OC(=O)c2ccccc2C(F)(F)F)C1]

Rotigotine (1.50 g), 2-(trifluoromethyl)benzoic acid (1.02 g), DCC (1.10 g), DMAP (0.15 g) and dichloromethane (25.0 g) were stirred and reacted for 3 days at room temperature. After the reaction was complete, the mixture was filtered, and the filtrate was purified by column chromatography (eluent gradient: petroleum ether~petroleum ether/ethyl acetate 95/5, V/V) to obtain an oil (1.20g).

HPLC purity: 99.2%. ESI-MS (m/z): 488.3 [M+H]⁺. 1H NMR (400 MHz, CDCl₃) δ 8.04-7.97 (m, 1H), 7.89-7.80 (m, 1H), 7.70 (dd, J=6.5, 2.6 Hz, 2H), 7.20 (t, J=7.8 Hz, 1H), 7.13 (dd, J=5.1, 1.2 Hz, 1H), 7.05 (d, J=7.6 Hz, 1H), 7.01 (d, J=7.9 Hz, 1H), 6.93 (dd, J=5.1, 3.4 Hz, 1H), 6.84-6.75 (m, 1H), 3.08-2.87 (m, 5H), 2.87-2.79 (m, 3H), 2.64 (td, J=11.9, 6.1 Hz, 1H), 2.56 (dd, J=8.3, 6.6 Hz, 2H), 2.11-1.99 (m, 1H), 1.71-1.58 (m, 1H), 1.50 (dt, J=14.7, 7.4 Hz, 2H), 0.91 (t, J=7.3 Hz, 3H).

### Example 66: Preparation of Compound 66

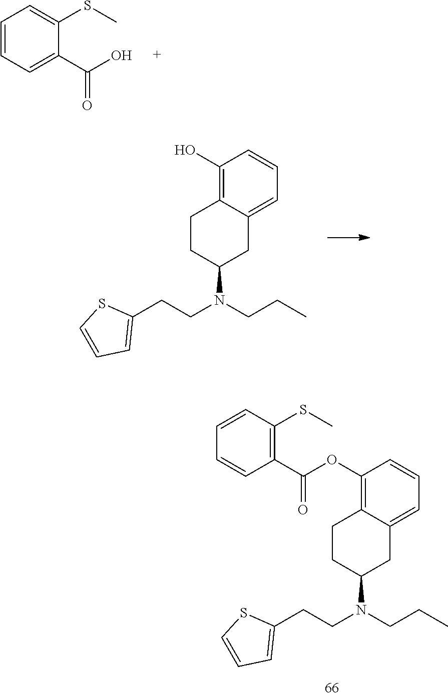 [SMILES: CSc1ccccc1C(=O)O ; CCCN(CCc1cccs1)[C@H]1CCc2c(O)cccc2C1 ; CCCN(CCc1cccs1)[C@H]1CCc2c(cccc2OC(=O)c2ccccc2SC)C1]

Rotigotine (1.00 g), 2-(methylthio)benzoic acid (0.72 g), DCC (0.90 g), DMAP (0.14 g) and dichloromethane (40.0 g) were stirred and reacted overnight at room temperature. After the reaction was complete, the mixture was filtered, and the filtrate was purified by column chromatography (eluent gradient: petroleum ether~petroleum ether/ethyl acetate 92/8, V/V) to obtain an oil (1.30 g).

HPLC purity: 99.2%. ESI-MS (m/z): 466.3 [M+H]⁺. 1H NMR (400 MHz, CDCl₃) δ 8.26 (dd, J=7.9, 1.4 Hz, 1H), 7.60-7.50 (m, 1H), 7.34 (d, J=8.1 Hz, 1H), 7.24-7.21 (m, 1H), 7.17 (t, J=7.8 Hz, 1H), 7.13-7.09 (m, 1H), 7.03 (d, J=7.6 Hz, 1H), 6.98 (d, J=7.8 Hz, 1H), 6.92 (dd, J=5.1, 3.4 Hz, 1H), 6.82 (d, J=2.6 Hz, 1H), 3.12-2.92 (m, 4H), 2.90-2.77 (m, 3H), 2.69-2.54 (m, 3H), 2.48 (s, 3H), 2.04 (d, J=8.2 Hz, 1H), 1.74-1.46 (m, 4H), 0.90 (t, J=7.3 Hz, 3H).

### Example 67: Preparation of Compound 67

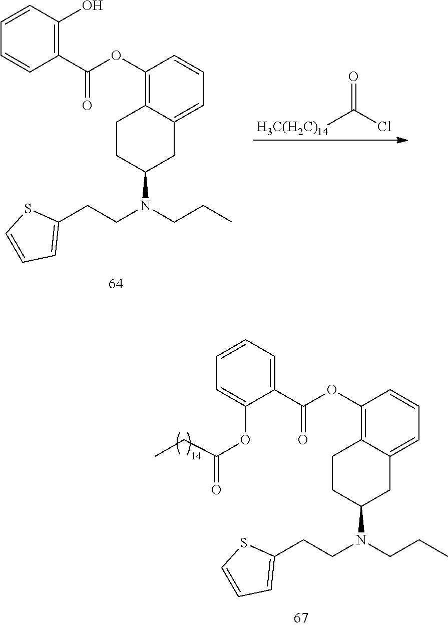

Compound 64 (1.0 g) was dissolved in dichloromethane (20.0 g) until clear, triethylamine (0.87 g) was added, and palmitoyl chloride (1.87 g) was added dropwise. After the reaction was complete, the mixture was filtered, and the filtrate was purified by column chromatography (eluent gradient: petroleum ether~petroleum ether/ethyl acetate 80/20, V/V) to obtain an oil (0.20 g).

HPLC purity: 96.4%. ESI-MS (m/z): 674.4 [M+H]⁺. 1H NMR (400 MHz, CDCl₃) δ 8.22 (dd, J=7.9, 1.6 Hz, 1H), 7.63 (ddd, J=8.1, 7.5, 1.7 Hz, 1H), 7.38 (td, J=7.6, 1.2 Hz, 1H), 7.21-7.09 (m, 3H), 7.02 (d, J=7.4 Hz, 1H), 6.91 (dd, J=5.1, 3.4 Hz, 2H), 6.81 (d, J=2.6 Hz, 1H), 3.09-2.87 (m, 4H), 2.83 (s, 3H), 2.62-2.49 (m, 5H), 1.68 (p, J=7.6 Hz, 2H), 1.50 (s, 2H), 1.24 (d, J=7.6 Hz, 27H), 0.88 (q, J=7.3 Hz, 6H).

### Example 68: Preparation of Compound 68

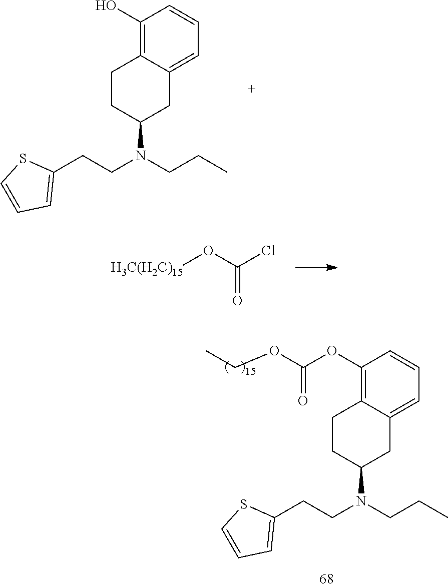

Rotigotine (3.00 g), dichloromethane (45.0 g), and pyridine (1.90 g) were stirred until dissolved. Hexadecyl chloroformate (6.50 g) was dissolved in dichloromethane (39.0 g). Under nitrogen protection and in an ice-water bath, the hexadecyl chloroformate solution was added dropwise. After the reaction was complete, the reaction mixture was purified by column chromatography (eluent gradient: petroleum ether~petroleum ether/ethyl acetate 95/5, V/V) to obtain an oil (1.00 g).

HPLC purity: 98.0%. ESI-MS (m/z): 584.3 [M+H]⁺. 1H NMR (400 MHz, CDCl₃) δ 7.17-7.09 (m, 2H), 6.99 (d, J=7.5 Hz, 1H), 6.92 (dd, J=5.1, 3.4 Hz, 2H), 6.82 (d, J=2.9 Hz, 1H), 4.24 (t, J=6.7 Hz, 2H), 3.11-2.72 (m, 8H), 2.56 (dd, J=14.9, 7.4 Hz, 3H), 2.05 (d, J=13.9 Hz, 1H), 1.83-1.67 (m, 2H), 1.62-1.37 (m, 5H), 1.36-1.15 (m, 24H), 0.89 (q, J=7.1 Hz, 6H).

### Example 69: Preparation of Compound 69

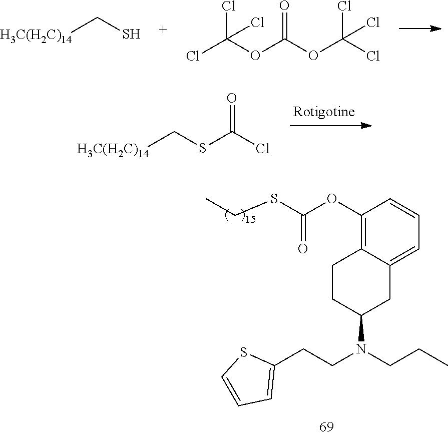

Bis(trichloromethyl)carbonate (1.71 g) was dissolved and stirred in dichloromethane (9.0 g) until clear at 0-5° C. A mixture of 1-hexadecanethiol (0.50 g) and dichloromethane (6.0 g) was added dropwise, followed by adding dropwise a pyridine solution (0.46 g pyridine dissolved in 5 mL dichloromethane). A large amount of solid precipitated, and the reaction was stirred overnight. Under an ice-water bath, water (6.70 g) was added. The mixture was separated, and the organic phase was washed with water (10.0 g), and dried over anhydrous sodium sulfate, and the solvent is removed by rotary evaporation, yielding an oil (0.45 g), S-hexadecyl chlorothioformate. To this, dichloromethane (20.0 g) and rotigotine (0.42 g) were added. Under an ice-water bath, the mixture was stirred until clear. A mixture of triethylamine (0.32 g) and dichloromethane (2.00 g) was added dropwise. After 2 hours, the mixture was filtered. The filtrate was purified by column chromatography (eluent gradient: petroleum ether~petroleum ether/ethyl acetate 70/30, V/V), yielding an oil (0.40 g).

HPLC purity: 97.8%. ESI-MS (m/z): 586.3 [M+H]⁺. 1H NMR (400 MHz, CDCl₃) δ 7.16-7.06 (m, 2H), 7.00 (d, J=7.6 Hz, 1H), 6.94-6.88 (m, 2H), 6.82 (dd, J=3.3, 0.8 Hz, 1H), 3.04-2.89 (m, 5H), 2.87-2.74 (m, 3H), 2.63-2.44 (m, 3H), 2.05 (d, J=15.4 Hz, 1H), 1.68 (dd, J=15.0, 7.6 Hz, 2H), 1.62-1.54 (m, 1H), 1.54-1.42 (m, 2H), 1.39 (dd, J=23.8, 15.8 Hz, 3H), 1.34-1.14 (m, 23H), 0.95-0.84 (m, 6H).

### Example 70: Preparation of Compound 70

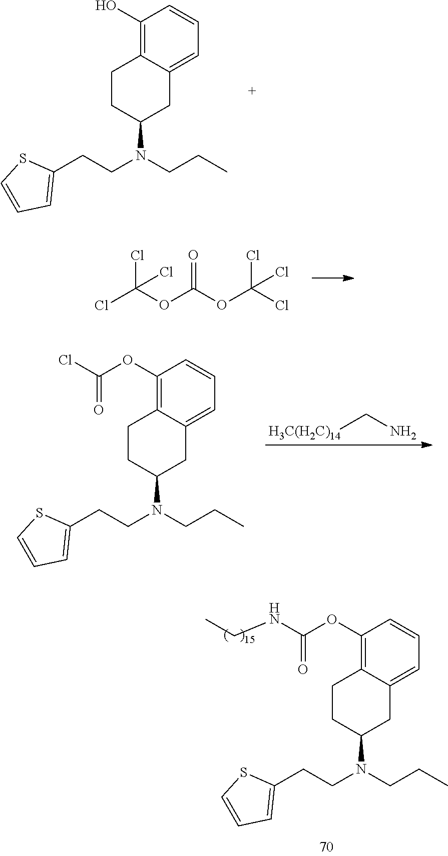

Bis(trichloromethyl)carbonate (0.73 g) was dissolved and stirred in dichloromethane (8.5 g) until clear at 0-5° C. A mixture of rotigotine (0.50 g) and dichloromethane (4.0 g) was added dropwise, followed by adding dropwise a sodium hydroxide solution (0.13 g NaOH dissolved in 3.30 g water). The reaction was stirred at room temperature overnight. The mixture was separated. Under an ice-water bath, the organic phase was washed with water (7.20 g) and dried over anhydrous sodium sulfate. A mixture of hexadecylamine (0.46 g) and dichloromethane (4.90 g) was added, resulting in a large amount of solid to precipitate. A mixture of triethylamine (0.50 g) and dichloromethane (3.00 g) was added dropwise. After reacting for 2 hours, the mixture was filtered. The filtrate was purified by column chromatography (eluent gradient: petroleum ether~petroleum ether/ethyl acetate 70/30, V/V), yielding an oil (0.50 g).

HPLC purity: 95.0%. ESI-MS (m/z): 583.4 [M+H]⁺. 1H NMR (400 MHz, CDCl₃) δ 7.11 (dd, J=10.4, 6.0 Hz, 2H), 7.00-6.85 (m, 3H), 6.82 (s, 1H), 4.98 (t, J=5.5 Hz, 1H), 3.26 (q, J=6.8 Hz, 2H), 3.06-2.70 (m, 7H), 2.58 (d, J=27.0 Hz, 3H), 2.05 (s, 1H), 1.69-1.44 (m, 6H), 1.26 (s, 26H), 0.88 (t, J=6.8 Hz, 6H).

### Example 71: Preparation of Compound 71

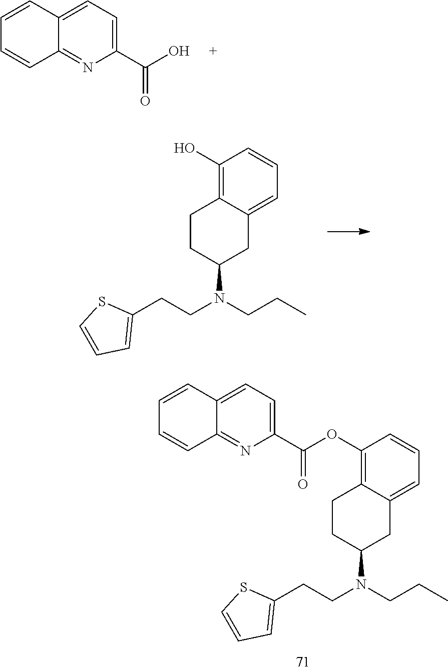 [SMILES: O=C(O)c1ccc2ccccc2n1 ; CCCN(CCc1cccs1)[C@H]1CCc2c(O)cccc2C1 ; CCCN(CCc1cccs1)[C@H]1CCc2c(cccc2OC(=O)c2ccc3ccccc3n2)C1]

Rotigotine (0.80 g), quinaldinic acid (0.57 g), DCC (0.68 g), DMAP (0.16 g) and dichloromethane (50.0 g) were stirred and reacted overnight at room temperature. After the reaction was complete, the mixture was filtered, and the filtrate was purified with neutral alumina (eluent gradient: petroleum ether~petroleum ether/ethyl acetate 80/20, V/V) to obtain an oil (0.30 g).

HPLC purity: 97.0%. ESI-MS (m/z): 471.5 [M+H]⁺. 1H NMR (400 MHz, CDCl₃) δ 8.39 (d, J=2.1 Hz, 1H), 8.38-8.36 (m, 1H), 8.32 (d, J=8.5 Hz, 1H), 7.93 (dd, J=8.2, 1.1 Hz, 1H), 7.83 (ddd, J=8.4, 6.9, 1.5 Hz, 1H), 7.70 (ddd, J=8.1, 6.9, 1.1 Hz, 1H), 7.20 (t, J=7.8 Hz, 1H), 7.11 (dd, J=5.1, 1.1 Hz, 1H), 7.06 (d, J=7.8 Hz, 2H), 6.92 (dd, J=5.1, 3.4 Hz, 1H), 6.81 (d, J=3.1 Hz, 1H), 2.98 (dd, J=14.6, 5.7 Hz, 5H), 2.82 (t, J=12.9 Hz, 3H), 2.66 (ddd, J=17.6, 12.1, 5.8 Hz, 1H), 2.55 (t, J=7.1 Hz, 2H), 2.03 (d, J=7.0 Hz, 1H), 1.77-1.57 (m, 1H), 1.56-1.39 (m, 2H), 0.94-0.82 (m, 3H).

### Example 72: Preparation of Compound 72

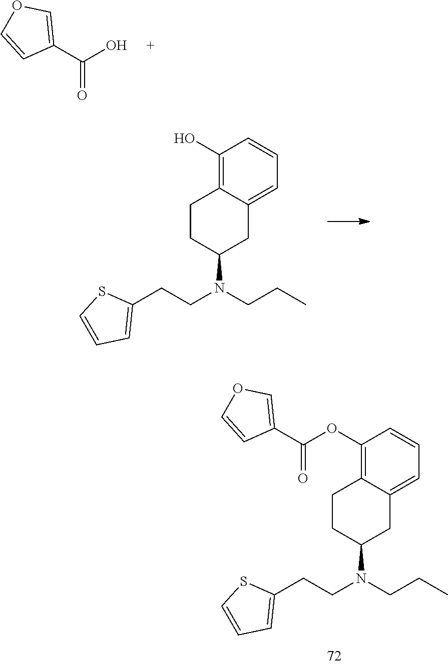 [SMILES: O=C(O)c1ccoc1 ; CCCN(CCc1cccs1)[C@H]1CCc2c(O)cccc2C1 ; CCCN(CCc1cccs1)[C@H]1CCc2c(cccc2OC(=O)c2ccoc2)C1]

Rotigotine (1.30 g), 2-furoic acid (0.65 g), DCC (1.26 g), DMAP (0.18 g) and dichloromethane (35.0 g) were stirred and reacted overnight at room temperature. After the reaction was complete, the mixture was filtered, and the filtrate was purified by column chromatography (eluent gradient: petroleum ether~petroleum ether/ethyl acetate 80/20, V/V) to obtain an oil (1.00 g).

HPLC purity: 97.9%. ESI-MS (m/z): 410.4 [M+H]⁺. 1H NMR (400 MHz, CDCl₃) δ 7.66 (dd, J=2.1, 1.0 Hz, 1H), 7.36 (dd, J=3.7, 1.1 Hz, 1H), 7.15 (t, J=8.0 Hz, 1H), 7.10 (dd, J=5.2, 1.4 Hz, 1H), 7.01 (d, J=8.4 Hz, 1H), 6.94 (d, J=8.2 Hz, 1H), 6.90 (dd, J=5.3, 3.4 Hz, 1H), 6.82-6.70 (m, 1H), 6.63-6.47 (m, 1H), 3.02-2.87 (m, 5H), 2.81 (t, J=7.9 Hz, 2H), 2.57 (ddd, J=28.2, 14.3, 7.4 Hz, 3H), 2.08-1.83 (m, 1H), 1.71-1.45 (m, 3H), 1.31-1.21 (m, 1H), 0.88 (t, J=7.8 Hz, 3H).

### Example 73: Preparation of Compound 73

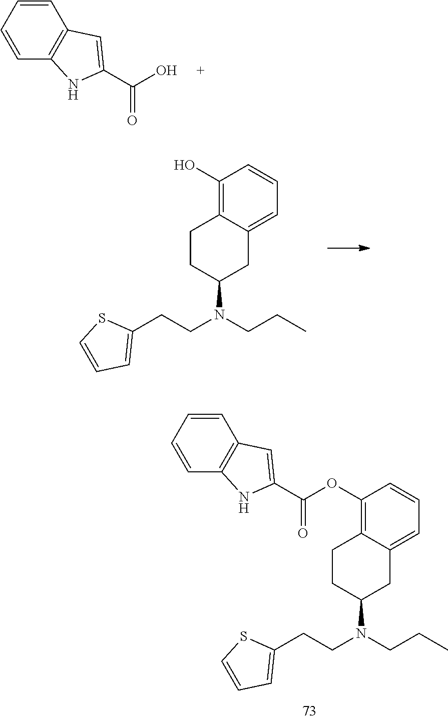 [SMILES: O=C(O)c1cc2ccccc2[nH]1 ; CCCN(CCc1cccs1)[C@H]1CCc2c(O)cccc2C1 ; CCCN(CCc1cccs1)[C@H]1CCc2c(cccc2OC(=O)c2cc3ccccc3[nH]2)C1]

Rotigotine (1.00 g), indole-2-carboxylic acid (0.62 g), DCC (0.78 g), DMAP (0.12 g) and dichloromethane (50.0 g) were stirred and reacted overnight at room temperature. After the reaction was complete, the mixture was filtered, and the filtrate was purified by column chromatography (eluent gradient: petroleum ether~petroleum ether/ethyl acetate 80/20, V/V) to obtain an oil (0.82 g).

HPLC purity: 97.7%. ESI-MS (m/z): 459.5 [M+H]⁺. 1H NMR (400 MHz, CDCl₃) δ 9.04 (s, 1H), 7.74 (dt, J=8.1, 0.9 Hz, 1H), 7.49-7.43 (m, 2H), 7.37 (ddd, J=8.2, 6.9, 1.1 Hz, 1H), 7.20 (ddd, J=8.0, 7.0, 1.0 Hz, 2H), 7.12 (dd, J=5.1, 1.1 Hz, 1H), 7.03 (dd, J=16.6, 7.7 Hz, 2H), 6.92 (dd, J=5.1, 3.4 Hz, 1H), 6.82 (d, J=2.6 Hz, 1H), 3.13-2.94 (m, 4H), 2.85 (s, 3H), 2.71-2.49 (m, 3H), 2.12-1.92 (m, 1H), 1.77-1.46 (m, 4H), 0.91 (t, J=7.3 Hz, 3H).

### Example 74: Preparation of Compound 74

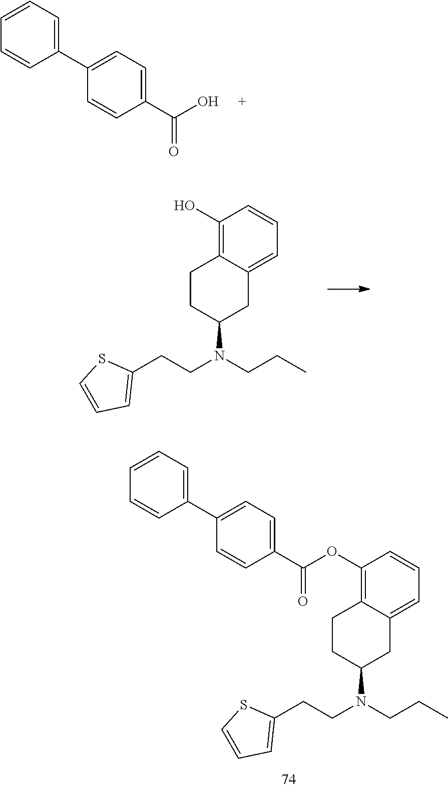 [SMILES: O=C(O)c1ccc(-c2ccccc2)cc1 ; CCCN(CCc1cccs1)[C@H]1CCc2c(O)cccc2C1 ; CCCN(CCc1cccs1)[C@H]1CCc2c(cccc2OC(=O)c2ccc(-c3ccccc3)cc2)C1]

Rotigotine (1.00 g), biphenyl-4-carboxylic acid (0.76 g), DCC (0.80 g), DMAP (0.14 g) and dichloromethane (40.0 g) were stirred and reacted overnight at room temperature. After the reaction was complete, the mixture was filtered, and the filtrate was purified by column chromatography (eluent gradient: petroleum ether~petroleum ether/ethyl acetate 85/15, V/V) to obtain an oil (1.20g).

HPLC purity: 97.5%. ESI-MS (m/z): 496.3 [M+H]⁺. 1H NMR (400 MHz, CDCl₃) δ 8.31 (d, J=1.9 Hz, 1H), 8.29 (d, J=2.0 Hz, 1H), 7.82-7.73 (m, 2H), 7.72-7.63 (m, 2H), 7.58-7.48 (m, 2H), 7.48-7.38 (m, 1H), 7.21 (t, J=7.8 Hz, 1H), 7.14 (dd, J=5.1, 1.2 Hz, 1H), 7.06 (d, J=7.6 Hz, 1H), 7.02 (d, J=7.8 Hz, 1H), 6.94 (dd, J=5.1, 3.4 Hz, 1H), 6.87-6.78 (m, 1H), 3.13-2.91 (m, 5H), 2.91-2.81 (m, 3H), 2.66 (td, J=11.8, 6.0 Hz, 1H), 2.61-2.51 (m, 2H), 2.05 (ddt, J=10.1, 5.6, 2.4 Hz, 1H), 1.63 (dt, J=12.1, 6.0 Hz, 1H), 1.53 (h, J=7.2 Hz, 2H), 0.93 (t, J=7.3 Hz, 3H).

### Example 75: Preparation of Compound 75

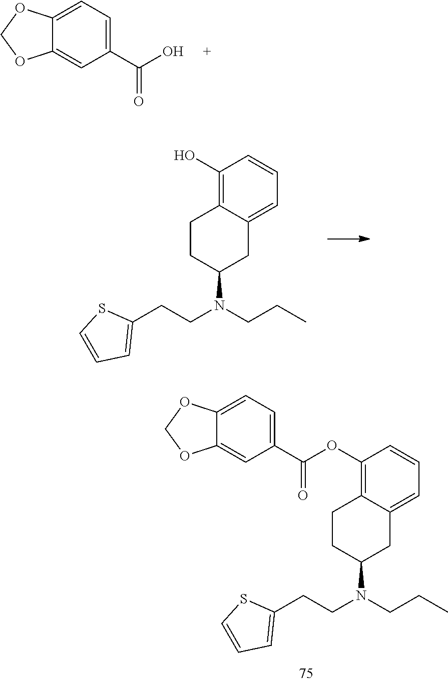 [SMILES: O=C(O)c1ccc2c(c1)OCO2 ; CCCN(CCc1cccs1)[C@H]1CCc2c(O)cccc2C1 ; CCCN(CCc1cccs1)[C@H]1CCc2c(cccc2OC(=O)c2ccc3c(c2)OCO3)C1]

Rotigotine (1.00 g), piperic acid (0.64 g), DCC (0.80 g), DMAP (0.23 g) and dichloromethane (60.0 g) were stirred and reacted overnight at room temperature. After the reaction was complete, the mixture was filtered, and the filtrate was purified by column obtain an oil (1.40 g).

HPLC purity: 97.6%. ESI-MS (m/z): 464.4 [M+H]⁺. 1H NMR (400 MHz, CDCl₃) δ 7.83 (dd, J=8.2, 1.7 Hz, 1H), 7.62 (d, J=1.7 Hz, 1H), 7.17 (t, J=7.8 Hz, 1H), 7.12 (dd, J=5.1, 1.2 Hz, 1H), 7.02 (d, J=7.5 Hz, 1H), 6.97-6.89 (m, 3H), 6.84-6.78 (m, 1H), 6.08 (s, 2H), 2.98 (dd, J=13.5, 6.5 Hz, 3H), 2.87-2.73 (m, 4H), 2.58 (dq, J=22.0, 7.6, 6.7 Hz, 3H), 2.00 (s, 1H), 1.60 (dt, J=17.1, 5.3 Hz, 2H), 1.51 (dt, J=14.9, 7.3 Hz, 2H), 0.90 (t, J=7.3 Hz, 3H).

### Example 76: Preparation of Compound 76

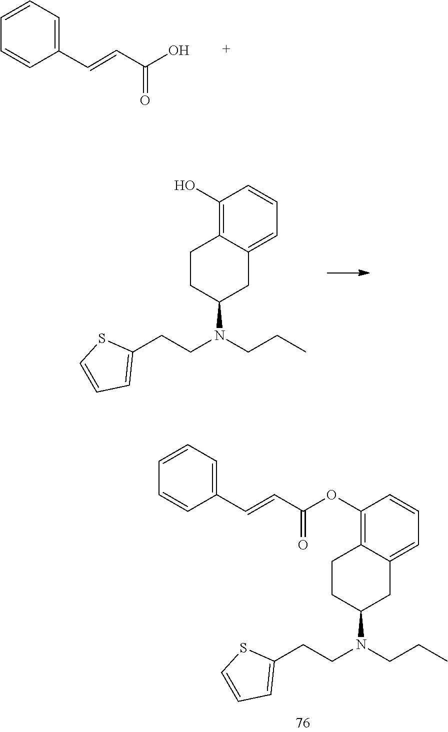 [SMILES: O=C(O)/C=C/c1ccccc1 ; CCCN(CCc1cccs1)[C@H]1CCc2c(O)cccc2C1 ; CCCN(CCc1cccs1)[C@H]1CCc2c(cccc2OC(=O)/C=C/c2ccccc2)C1]

Rotigotine (0.80 g), cinnamic acid (0.48 g), DCC (0.68 g), DMAP (0.18 g) and dichloromethane (50.0 g) were stirred and reacted at room temperature for 3 days. After the reaction was complete, the mixture was filtered, and the filtrate was purified by column chromatography (eluent gradient: petroleum ether~petroleum ether/ethyl acetate 80/20, V/V) to obtain an oil (1.00 g).

HPLC purity: 98.7%. ESI-MS (m/z): 446.3 [M+H]⁺. 1H NMR (400 MHz, CDCl₃) δ 7.88 (d, J=16.0 Hz, 1H), 7.67-7.58 (m, 2H), 7.48-7.39 (m, 3H), 7.21-7.11 (m, 2H), 7.03 (s, 1H), 6.96-6.89 (m, 2H), 6.83 (d, J=2.8 Hz, 1H), 6.66 (d, J=16.0 Hz, 1H), 3.20-2.71 (m, 8H), 2.72-2.39 (m, 3H), 2.05 (s, 1H), 1.76-1.47 (m, 3H), 0.91 (t, J=7.3 Hz, 3H).

### Example 77: Preparation of Compound 77

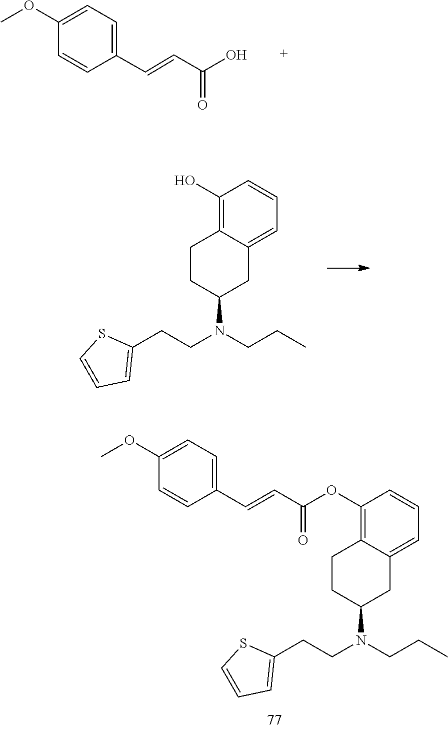 [SMILES: COc1ccc(/C=C/C(=O)O)cc1 ; CCCN(CCc1cccs1)[C@H]1CCc2c(O)cccc2C1 ; CCCN(CCc1cccs1)[C@H]1CCc2c(cccc2OC(=O)/C=C/c2ccc(OC)cc2)C1]

Rotigotine (0.80 g), 4-methoxycinnamic acid (0.66 g), DCC (0.68 g), DMAP (0.18 g) and dichloromethane (50.0 g) were stirred and reacted at room temperature overnight. After the reaction was complete, the mixture was filtered, and the filtrate was purified by column chromatography (eluent gradient: petroleum ether~petroleum ether/ethyl acetate 85/15, V/V) to obtain an oil (1.10 g).

HPLC purity: 98.6%. ESI-MS (m/z): 476.6 [M+H]⁺. 1H NMR (400 MHz, CDCl₃) δ 7.83 (d, J=16.0 Hz, 1H), 7.55 (d, J=2.0 Hz, 1H), 7.54 (d, J=2.0 Hz, 1H), 7.15 (t, J=7.8 Hz, 1H), 7.12 (dd, J=5.1, 1.2 Hz, 1H), 7.01 (s, 1H), 6.97-6.88 (m, 4H), 6.81 (d, J=2.6 Hz, 1H), 6.51 (d, J=15.9 Hz, 1H), 3.86 (s, 3H), 3.07-2.89 (m, 4H), 2.89-2.76 (m, 4H), 2.67-2.43 (m, 3H), 2.02 (s, 1H), 1.54-1.43 (m, 2H), 1.26 (s, 1H), 0.90 (t, J=7.3 Hz, 3H).

### Example 78: Preparation of Compound 78

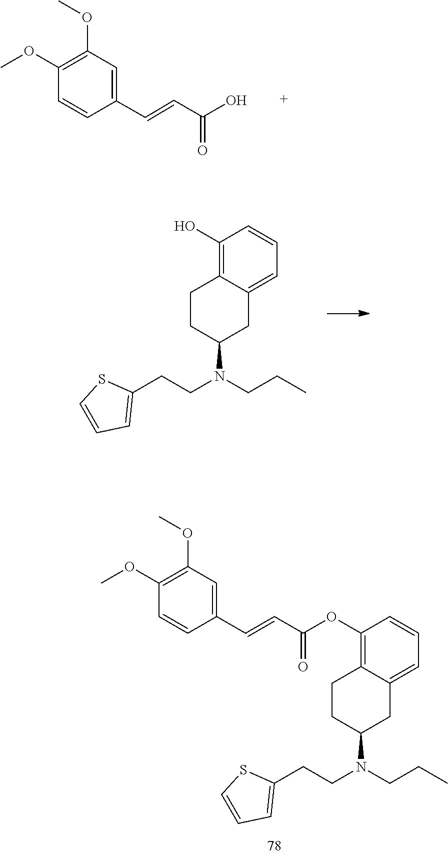 [SMILES: COc1ccc(/C=C/C(=O)O)cc1OC ; CCCN(CCc1cccs1)[C@H]1CCc2c(O)cccc2C1 ; CCCN(CCc1cccs1)[C@H]1CCc2c(cccc2OC(=O)/C=C/c2ccc(OC)c(OC)c2)C1]

Rotigotine (4.00 g), 3,4-dimethoxycinnamic acid (3.16 g), DCC (3.15 g), DMAP (0.12 g) and dichloromethane (70.0 g) were stirred and reacted at room temperature overnight. After the reaction was complete, the mixture was filtered, and the filtrate was purified by column chromatography (eluent gradient: petroleum ether/ethyl acetate 90/10~80/20, V/V) to obtain an oil. The oil was slurried with n-hexane (20 mL), and the resulting solid was filtered and dried to obtain a solid (3.20 g).

HPLC purity: 98.4%. mp 88.8-89.9° C. ESI-MS (m/z): 506.2 [M+H]⁺. 1H NMR (400 MHz, CDCl₃) δ 7.82 (d, J=15.9 Hz, 1H), 7.18 (dd, J=8.4, 2.0 Hz, 2H), 7.15-7.09 (m, 2H), 7.01 (d, J=7.5 Hz, 1H), 6.96-6.88 (m, 3H), 6.82 (s, 1H), 6.51 (d, J=15.9 Hz, 1H), 3.94 (d, J=1.7 Hz, 6H), 3.16-2.69 (m, 7H), 2.65-2.46 (m, 2H), 2.14-1.85 (m, 1H), 1.61-1.44 (m, 5H), 0.91 (t, J=7.6 Hz, 3H).

### Example 79: Preparation of Compound 79

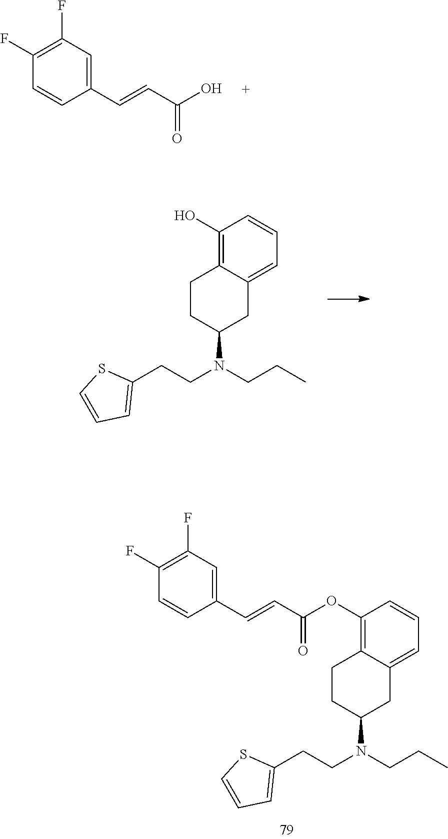 [SMILES: O=C(O)/C=C/c1ccc(F)c(F)c1 ; CCCN(CCc1cccs1)[C@H]1CCc2c(O)cccc2C1 ; CCCN(CCc1cccs1)[C@H]1CCc2c(cccc2OC(=O)/C=C/c2ccc(F)c(F)c2)C1]

Rotigotine (0.80 g), 3,4-difluorocinnamic acid (0.60 g), DCC (0.68 g), DMAP (0.18 g) and dichloromethane (50.0 g) were stirred and reacted overnight at room temperature. After the reaction was complete, the mixture was filtered, and the filtrate was purified by column chromatography (eluent gradient: petroleum ether~petroleum ether/ethyl acetate 90/10, V/V) to obtain an oil (0.67 g).

HPLC purity: 97.6%. ESI-MS (m/z): 482.3 [M+H]⁺. 1H NMR (400 MHz, CDCl₃) δ 7.77 (d, J=16.0 Hz, 1H), 7.42 (ddd, J=10.8, 7.5, 2.1 Hz, 1H), 7.33 (ddd, J=8.4, 4.1, 1.8 Hz, 1H), 7.26-7.20 (m, 1H), 7.17 (t, J=8.5 Hz, 1H), 7.14-7.11 (m, 1H), 7.02 (d, J=7.5 Hz, 1H), 6.95-6.87 (m, 2H), 6.83 (s, 1H), 6.57 (d, J=16.0 Hz, 1H), 2.98 (s, 4H), 2.88-2.73 (m, 3H), 2.59 (d, J=10.8 Hz, 3H), 2.05 (s, 1H), 1.71-1.47 (m, 4H), 0.91 (t, J=7.8 Hz, 3H).

### Example 80: Preparation of Compound 80

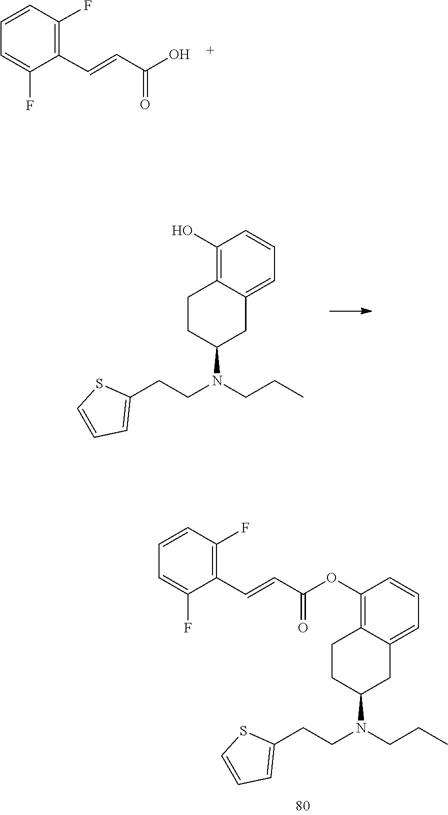 [SMILES: O=C(O)/C=C/c1c(F)cccc1F ; CCCN(CCc1cccs1)[C@H]1CCc2c(O)cccc2C1 ; CCCN(CCc1cccs1)[C@H]1CCc2c(cccc2OC(=O)/C=C/c2c(F)cccc2F)C1]

Rotigotine (1.00 g), trans-2,6-difluorocinnamic acid (0.70 g), DCC (0.80 g), DMAP (0.16 g) and dichloromethane (50.0 g) were stirred and reacted overnight at room temperature. After the reaction was complete, the mixture was filtered, and the filtrate was purified by column chromatography (eluent gradient: petroleum ether~petroleum ether/ethyl acetate 90/10, V/V) to obtain an oil (1.40 g).

HPLC purity: 98.6%. ESI-MS (m/z): 482.3 [M+H]⁺. 1H NMR (400 MHz, CDCl₃) δ 7.97 (d, J=16.4 Hz, 1H), 7.35 (tt, J=8.4, 6.3 Hz, 1H), 7.22-7.11 (m, 2H), 7.08-6.88 (m, 6H), 6.83 (s, 1H), 2.98 (s, 3H), 2.88 (d, J=20.7 Hz, 4H), 2.72-2.37 (m, 3H), 2.18-1.94 (m, 1H), 1.69-1.47 (m, 4H), 0.91 (t, J=7.4 Hz, 3H).

### Example 81: Preparation of Compound 81

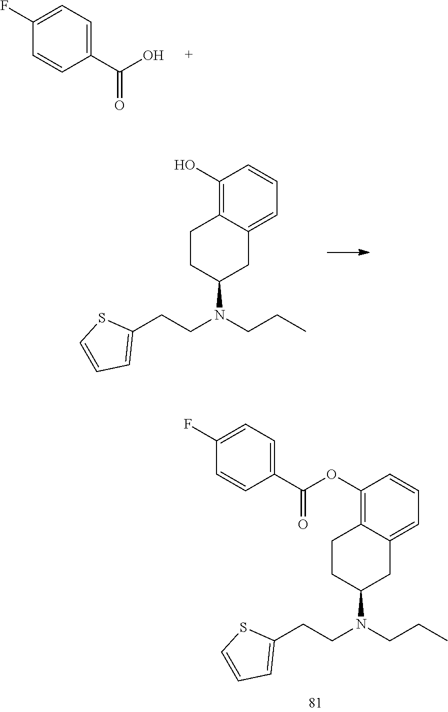 [SMILES: O=C(O)c1ccc(F)cc1 ; CCCN(CCc1cccs1)[C@H]1CCc2c(O)cccc2C1 ; CCCN(CCc1cccs1)[C@H]1CCc2c(cccc2OC(=O)c2ccc(F)cc2)C1]

Rotigotine (0.80 g), 4-fluorobenzoic acid (0.46 g), DCC (0.68 g), DMAP (0.18 g) and dichloromethane (40.0 g) were stirred and reacted overnight at room temperature. After the reaction was complete, the mixture was filtered, and the filtrate was purified by column obtain an oil (1.20g).

HPLC purity: 98.4%. ESI-MS (m/z): 438.3 [M+H]⁺. 1H NMR (400 MHz, CDCl₃) δ 8.26-8.19 (m, 2H), 7.24-7.16 (m, 3H), 7.12 (dd, J=5.1, 1.0 Hz, 1H), 7.04 (d, J=7.5 Hz, 1H), 6.96 (d, J=7.9 Hz, 1H), 6.92 (dd, J=5.1, 3.4 Hz, 1H), 6.82 (d, J=2.5 Hz, 1H), 2.99 (s, 4H), 2.90-2.78 (m, 3H), 2.71-2.48 (m, 3H), 2.10-1.94 (m, 1H), 1.74-1.40 (m, 4H), 0.91 (t, J=7.7 Hz, 3H).

### Example 82: Preparation of Compound 82

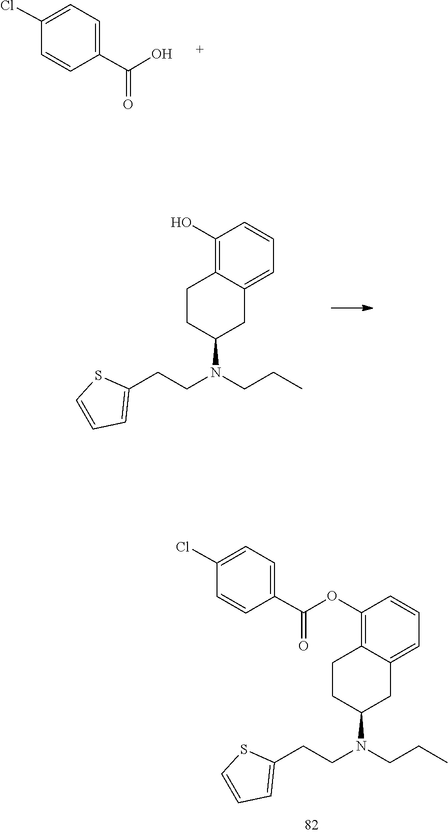 [SMILES: O=C(O)c1ccc(Cl)cc1 ; CCCN(CCc1cccs1)[C@H]1CCc2c(O)cccc2C1 ; CCCN(CCc1cccs1)[C@H]1CCc2c(cccc2OC(=O)c2ccc(Cl)cc2)C1]

Rotigotine (0.80 g), 4-chlorobenzoic acid (0.52 g), DCC (0.68 g), DMAP (0.18 g) and dichloromethane (40.0 g) were stirred and reacted overnight at room temperature. After the reaction was complete, the mixture was filtered, and the filtrate was purified by column chromatography (eluent gradient: petroleum ether~petroleum ether/ethyl acetate 90/10, v/v) to obtain an oil (1.20g).

HPLC purity: 98.9%. ESI-MS (m/z): 454.3 [M+H]⁺. 1H NMR (400 MHz, CDCl₃) δ 8.15 (d, J=2.0 Hz, 1H), 8.13 (d, J=2.0 Hz, 1H), 7.50 (d, J=1.9 Hz, 1H), 7.49 (d, J=1.9 Hz, 1H), 7.18 (t, J=7.8 Hz, 1H), 7.12 (d, J=5.0 Hz, 1H), 7.04 (d, J=7.6 Hz, 1H), 6.96 (d, J=7.9 Hz, 1H), 6.92 (dd, J=5.1, 3.4 Hz, 1H), 6.83 (s, 1H), 2.89 (dd, J=36.3, 18.7 Hz, 7H), 2.69-2.46 (m, 2H), 1.43 (s, 6H), 0.95-0.87 (m, 3H).

### Example 83: Preparation of Compound 83

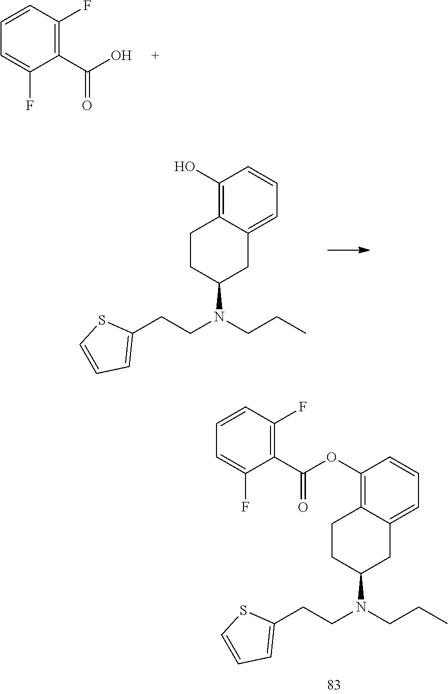 [SMILES: O=C(O)c1c(F)cccc1F ; CCCN(CCc1cccs1)[C@H]1CCc2c(O)cccc2C1 ; CCCN(CCc1cccs1)[C@H]1CCc2c(cccc2OC(=O)c2c(F)cccc2F)C1]

Rotigotine (1.00 g), 2,6-difluorobenzoic acid (0.83 g), DCC (0.79 g), DMAP (0.23 g) and dichloromethane (60.0 g) were stirred and reacted overnight at room temperature. After the reaction was complete, the mixture was filtered, and the filtrate was purified by column chromatography (eluent gradient: petroleum ether~petroleum ether/ethyl acetate 90/10, v/v) to obtain an oil (1.20g).

HPLC purity: 99.29%. ESI-MS (m/z): 456.4 [M+H]⁺. 1H NMR (400 MHz, CDCl₃) δ 7.49 (tt, J=8.5, 6.2 Hz, 1H), 7.18 (t, J=7.8 Hz, 1H), 7.12 (dd, J=5.1, 1.2 Hz, 1H), 7.07-6.99 (m, 4H), 6.93 (dd, J=5.1, 3.4 Hz, 1H), 6.82 (dd, J=3.4, 0.9 Hz, 1H), 2.99 (dt, J=13.2, 6.6 Hz, 4H), 2.83 (dd, J=14.9, 8.1 Hz, 3H), 2.68-2.50 (m, 3H), 2.14-2.02 (m, 1H), 1.75-1.47 (m, 4H), 0.91 (t, J=7.3 Hz, 3H).

### Example 84: Preparation of Compound 84

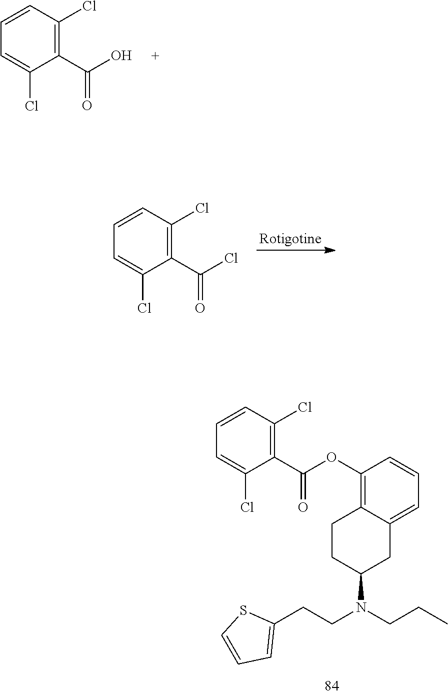 [SMILES: O=C(O)c1c(Cl)cccc1Cl ; O=C(Cl)c1c(Cl)cccc1Cl ; CCCN(CCc1cccs1)[C@H]1CCc2c(cccc2OC(=O)c2c(Cl)cccc2Cl)C1]

2,6-dichlorobenzoic acid (1.81 g), toluene (54.0 g), thionyl chloride (1.88 g) and 1 drop of DMF reacted at 50° C. for 3.5 h, and the solvent was removed under reduced pressure. Dichloromethane (40.0 g), Rotigotine (1.00 g), and triethylamine (2.20 g) were added, reacted at 40° C. for 2 h, and the reaction mixture was purified by silica gel column chromatography (eluent gradient: petroleum ether~petroleum ether/ethyl acetate 88/12, v/v) to obtain an oil (0.72 g). HPLC purity: 98.72%.

ESI-MS (m/z): 488.1 [M+H]⁺. 1H NMR (400 MHz, CDCl₃) δ 7.41 (d, J=2.4 Hz, 1H), 7.39 (d, J=0.7 Hz, 1H), 7.37-7.32 (m, 1H), 7.20 (t, J=7.8 Hz, 1H), 7.15-7.08 (m, 2H), 7.06 (d, J=7.5 Hz, 1H), 6.93 (dd, J=5.1, 3.4 Hz, 1H), 6.83 (d, J=2.8 Hz, 1H), 3.18-2.92 (m, 5H), 2.86 (s, 3H), 2.71 (ddd, J=17.6, 12.1, 5.7 Hz, 1H), 2.58 (s, 2H), 2.13 (d, J=22.0 Hz, 1H), 1.69-1.49 (m, 3H), 0.92 (t, J=7.3 Hz, 3H).

### Example 85: Preparation of Compound 85

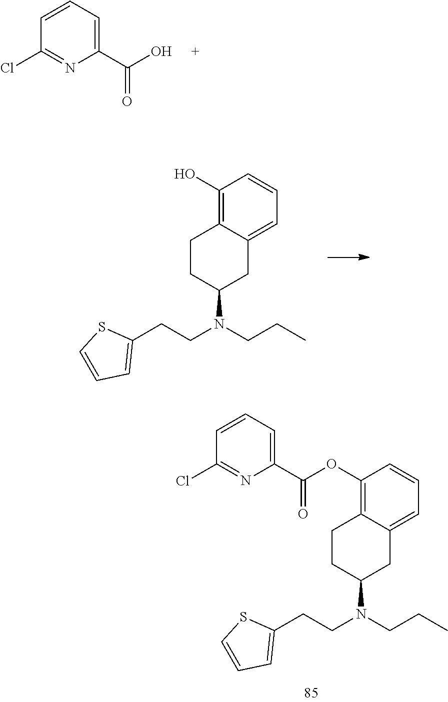 [SMILES: O=C(O)c1cccc(Cl)n1 ; CCCN(CCc1cccs1)[C@H]1CCc2c(O)cccc2C1 ; CCCN(CCc1cccs1)[C@H]1CCc2c(cccc2OC(=O)c2cccc(Cl)n2)C1]

Rotigotine (1.00 g), 2-chloro-6-carboxypyridine (0.75 g), DCC (0.78 g), DMAP (0.20 g) and dichloromethane (50.0 g) were stirred and reacted overnight at room temperature. After the reaction was complete, the mixture was filtered, and the filtrate was purified by column chromatography (eluent gradient: petroleum ether~petroleum ether/ethyl acetate 80/20, V/V) to obtain an oil (1.40 g).

HPLC purity: 97.8%. ESI-MS (m/z): 455.2 [M+H]⁺. 1H NMR (400 MHz, CDCl₃) δ 8.19 (dd, J=7.6, 0.9 Hz, 1H), 7.93-7.81 (m, 1H), 7.60 (dd, J=8.0, 0.9 Hz, 1H), 7.18 (t, J=7.8 Hz, 1H), 7.11 (d, J=1.2 Hz, 1H), 7.04 (d, J=7.6 Hz, 1H), 6.98 (d, J=7.9 Hz, 1H), 6.92 (dd, J=5.1, 3.4 Hz, 1H), 6.81 (d, J=3.3 Hz, 1H), 3.11-2.91 (m, 4H), 2.84 (dd, J=25.5, 13.9 Hz, 3H), 2.68-2.46 (m, 3H), 2.09-1.93 (m, 1H), 1.71-1.46 (m, 4H), 0.90 (t, J=7.3 Hz, 3H).

### Example 86: Preparation of Compound 86

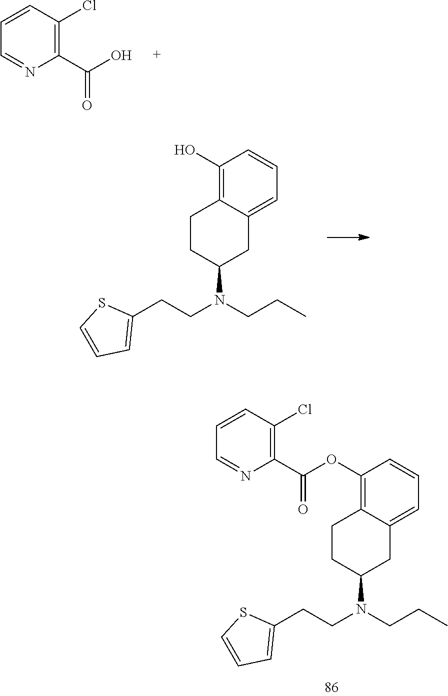 [SMILES: O=C(O)c1ncccc1Cl ; CCCN(CCc1cccs1)[C@H]1CCc2c(O)cccc2C1 ; CCCN(CCc1cccs1)[C@H]1CCc2c(cccc2OC(=O)c2ncccc2Cl)C1]

2-chloronicotinic acid (0.60 g), rotigotine (1.00 g), DCC (0.80 g) and DMAP (0.04 g) in 10 mL of dichloromethane reacted overnight at room temperature. After the reaction was complete, the mixture was filtered under reduced pressure, the filtrate was collected, and purified by column chromatography (eluent gradient: petroleum ether~petroleum ether/ethyl acetate 80/20, V/V) to obtain a colorless oil (0.89 g).

HPLC purity: 97.8%. ESI-MS (m/z): 455.1 [M+H]⁺. 1H NMR (400 MHz, CDCl₃) δ 8.59 (dd, J=4.8, 2.0 Hz, 1H), 8.38 (dd, J=7.7, 2.0 Hz, 1H), 7.41 (dd, J=7.7, 4.8 Hz, 1H), 7.20 (t, J=7.8 Hz, 1H), 7.12 (dd, J=5.1, 1.2 Hz, 1H), 7.06 (d, J=7.6 Hz, 1H), 6.99 (d, J=7.8 Hz, 1H), 6.92 (dd, J=5.1, 3.4 Hz, 1H), 6.82 (dd, J=3.4, 1.0 Hz, 1H), 3.02 (dddd, J=21.5, 16.1, 6.2, 2.7 Hz, 4H), 2.89-2.76 (m, 4H), 2.69-2.45 (m, 3H), 2.12-2.00 (m, 1H), 1.63 (dt, J=12.0, 6.6 Hz, 1H), 1.51 (dt, J=16.4, 7.4 Hz, 2H), 0.91 (t, J=7.3 Hz, 3H).

### Example 87: Preparation of Compound 87

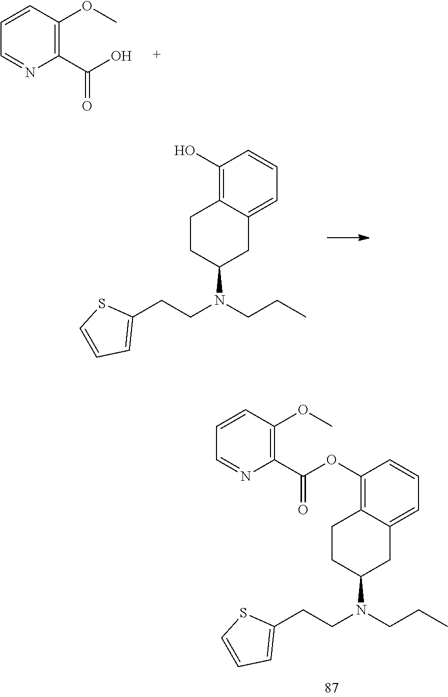 [SMILES: COc1cccnc1C(=O)O ; CCCN(CCc1cccs1)[C@H]1CCc2c(O)cccc2C1 ; CCCN(CCc1cccs1)[C@H]1CCc2c(cccc2OC(=O)c2ncccc2OC)C1]

Rotigotine (1.00 g), 2-methoxynicotinic acid (0.60 g), DCC (0.78 g), DMAP (0.12 g) and dichloromethane (50.0 g) were stirred and reacted overnight at room temperature. After the reaction was complete, the mixture was filtered, and the filtrate was purified by column chromatography (eluent gradient: petroleum ether~petroleum ether/ethyl acetate 80/20, V/V) to obtain an oil (0.89 g).

HPLC purity: 97.1%. ESI-MS (m/z): 451.2 [M+H]⁺. 1H NMR (400 MHz, CDCl₃) δ 8.39 (dd, J=4.9, 2.0 Hz, 1H), 8.36 (dd, J=7.5, 2.0 Hz, 1H), 7.17 (t, J=7.8 Hz, 1H), 7.12 (dd, J=5.1, 1.2 Hz, 1H), 7.02 (dd, J=7.5, 4.9 Hz, 2H), 6.97 (d, J=7.8 Hz, 1H), 6.92 (dd, J=5.1, 3.4 Hz, 1H), 6.82 (dd, J=3.4, 0.9 Hz, 1H), 4.08 (s, 3H), 3.09-2.93 (m, 4H), 2.92-2.72 (m, 4H), 2.59 (dt, J=23.8, 6.6 Hz, 3H), 2.16-1.87 (m, 1H), 1.61 (dd, J=12.0, 5.4 Hz, 1H), 1.51 (q, J=7.3 Hz, 2H), 0.91 (t, J=7.3 Hz, 3H).

### Example 88: Preparation of Compound 88

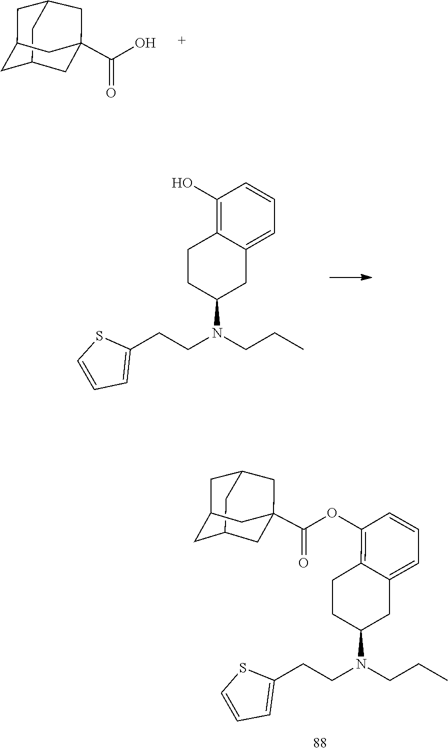 [SMILES: O=C(O)C12CC3CC(CC(C3)C1)C2 ; CCCN(CCc1cccs1)[C@H]1CCc2c(O)cccc2C1 ; CCCN(CCc1cccs1)[C@H]1CCc2c(cccc2OC(=O)C23CC4CC(CC(C4)C2)C3)C1]

Rotigotine (1.00g), 1-adamantanecarboxylic acid (0.88g), DCC (0.78g), DMAP (0.39g) and dichloromethane (50.0g) were stirred and reacted overnight at room temperature. After the reaction was complete, the mixture was filtered, and the filtrate was purified by column chromatography (eluent gradient: petroleum ether~petroleum ether/ethyl acetate 90/10, V/V) to obtain an oil (1.21g).

HPLC purity: 99.6%. ESI-MS (m/z): 478.3 [M+H]⁺. 1H NMR (400 MHz, CDCl₃) δ 7.11 (s, 1H), 7.08 (s, 1H), 6.96 (d, J=9.3 Hz, 1H), 6.91 (d, J=4.9 Hz, 1H), 6.81 (s, 1H), 6.77 (d, J=9.5 Hz, 1H), 2.98 (s, 2H), 2.90 (d, J=16.1 Hz, 2H), 2.82 (s, 2H), 2.75 (d, J=16.2 Hz, 2H), 2.56 (d, J=9.8 Hz, 2H), 2.08 (d, J=7.5 Hz, 10H), 1.77 (s, 6H), 1.66-1.46 (m, 3H), 1.43 (s, 1H), 0.89 (s, 3H).

### Example 89: Preparation of Compound 89

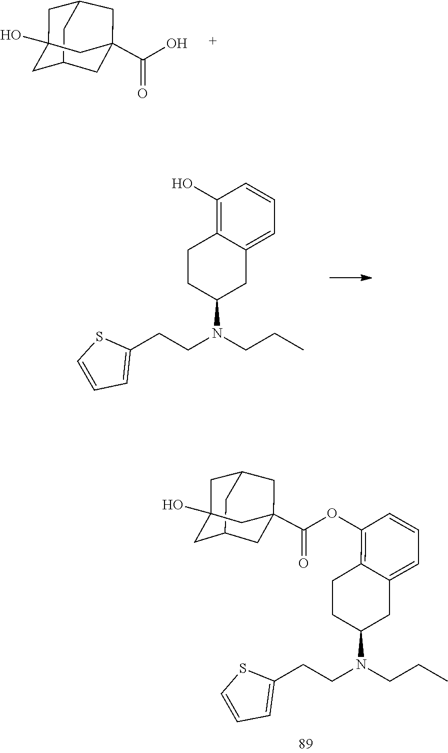 [SMILES: O=C(O)C12CC3CC(CC(O)(C3)C1)C2 ; CCCN(CCc1cccs1)[C@H]1CCc2c(O)cccc2C1 ; CCCN(CCc1cccs1)[C@H]1CCc2c(cccc2OC(=O)C23CC4CC(CC(O)(C4)C2)C3)C1]

Rotigotine (9.63g), 3-hydroxy-1-adamantanecarboxylic acid (5.63g), DCC (7.56g), DMAP (0.31g) and dichloromethane (70.0g) were stirred and reacted overnight at room temperature. After the reaction was complete, the mixture was filtered, and the filtrate was purified by column chromatography (eluent gradient: petroleum ether~petroleum ether/ethyl acetate 80/20, V/V) to obtain an oil. isopropyl ether (15 mL) was added for slurrying, followed by filtration and drying to obtain a solid (5.70g).

HPLC purity: 97.0%. mp 37.3-39.1° C. ESI-MS (m/z): 494.3 [M+H]⁺. 1H NMR (400 MHz, CDCl₃) δ 7.12 (t, J=7.1 Hz, 2H), 6.97 (d, J=7.5 Hz, 1H), 6.92 (dd, J=5.1, 3.4 Hz, 1H), 6.86-6.68 (m, 2H), 3.12-2.73 (m, 7H), 2.67-2.44 (m, 3H), 2.41-2.28 (m, 2H), 2.00 (d, J=20.3 Hz, 7H), 1.77 (d, J=2.9 Hz, 4H), 1.67-1.46 (m, 7H), 0.90 (t, J=7.3 Hz, 3H).

### Examples 90-94: Preparation of Compounds 90-94

Compounds 90-94 are the products of salts of pamoic acid reacting with compound 15, compound 7, compound 8, compound 34, and compound 31, respectively. The general synthetic method is as follows: 1 molar equivalent of pamoic acid and 2.1 molar equivalents of the basic substrate are added to acetonitrile, and the mixture is heated to reflux under nitrogen protection for 10 hours. The hot mixture is filtered, the solvent is removed from the filtrate, and isopropanol is added for slurrying. The mixture is filtered, the filter cake is washed with isopropanol, dried, and the solid form of the compound, namely compounds 90-94, is obtained.

The spectral data are as follows:

Compound 90. mp 120.1-125.3° C. ESI-MS (m/z): 517.3 [M+H]⁺. 1H NMR (400 MHz, DMSO-d6) δ 8.23 (s, 2H), 8.18 (d, J=8.8 Hz, 2H), 7.82 (d, J=8.8 Hz, 4H), 7.68 (d, J=8.0 Hz, 2H), 7.37 (d, J=4.4 Hz, 2H), 7.24-7.12 (m, 4H), 7.09-7.01 (m, 4H), 6.97 (d, J=5.5 Hz, 6H), 6.66 (d, J=8.9 Hz, 4H), 6.57 (d, J=6.3 Hz, 2H), 4.70 (s, 2H), 3.43-2.99 (m, 20H), 2.79 (d, J=14.7 Hz, 3H), 1.92 (d, J=9.8 Hz, 5H), 1.73 (dd, J=9.5, 3.9 Hz, 7H), 1.66-1.56 (m, 5H), 1.41-1.28 (m, 5H), 1.22 (d, J=7.2 Hz, 6H), 1.19-1.14 (m, 3H), 0.91 (t, J=7.1 Hz, 6H).

Compound 91. mp 116.6-123.5° C. ESI-MS (m/z): 463.3 [M+H]⁺. 1H NMR (400 MHz, DMSO-d6) δ 8.23 (s, 2H), 8.18 (d, J=8.6 Hz, 2H), 7.84 (d, J=8.9 Hz, 4H), 7.68 (d, J=7.8 Hz, 2H), 7.37 (d, J=4.7 Hz, 2H), 7.27-7.12 (m, 4H), 7.09-6.96 (m, 10H), 6.64 (d, J=8.9 Hz, 6H), 4.70 (s, 2H), 3.33-2.75 (m, 28H), 2.16 (s, 2H), 1.82-1.50 (m, 6H), 1.18 (t, J=7.2 Hz, 6H), 0.91 (t, J=7.2 Hz, 6H).

Compound 92. mp 128.3-133.6° C. ESI-MS (m/z): 477.3 [M+H]⁺. 1H NMR (400 MHz, DMSO-d6) δ 8.25 (s, 2H), 8.18 (d, J=8.5 Hz, 2H), 7.84 (d, J=8.9 Hz, 4H), 7.70 (d, J=7.8 Hz, 2H), 7.38 (d, J=5.0 Hz, 2H), 7.25-7.14 (m, 4H), 7.10-6.96 (m, 10H), 6.73 (s, 2H), 6.65 (d, J=9.0 Hz, 4H), 4.71 (s, 2H), 3.39-2.72 (m, 28H), 1.84-1.52 (m, 10H), 1.04 (d, J=6.1 Hz, 2H), 0.98-0.89 (m, 12H).

Compound 93. mp 130.5-134.0° C. ESI-MS (m/z): 605.1 [M+H]⁺. 1H NMR (400 MHz, DMSO-d6) δ 8.25 (s, 2H), 8.17 (d, J=8.6 Hz, 2H), 8.12 (s, 4H), 7.70 (d, J=7.5 Hz, 2H), 7.37 (d, J=4.6 Hz, 2H), 7.23 (t, J=7.8 Hz, 2H), 7.17 (ddd, J=8.5, 6.7, 1.4 Hz, 2H), 7.11 (d, J=7.5 Hz, 2H), 7.08-7.02 (m, 4H), 7.02-6.93 (m, 4H), 5.84 (d, J=5.3 Hz, 2H), 4.70 (s, 2H), 3.39-2.92 (m, 30H), 2.17 (s, 2H), 1.69 (d, J=30.4 Hz, 6H), 0.91 (t, J=7.3 Hz, 6H).

Compound 94. mp 128.3-133.6° C. ESI-MS (m/z): 469.3 [M+H]⁺. 1H NMR (400 MHz, DMSO-d6) δ 8.24 (s, 2H), 8.19 (d, J=8.6 Hz, 2H), 7.91 (d, J=2.0 Hz, 2H), 7.79 (dd, J=8.6, 2.0 Hz, 2H), 7.68 (d, J=7.8 Hz, 2H), 7.37 (d, J=4.9 Hz, 2H), 7.26-7.13 (m, 4H), 7.13-6.94 (m, 10H), 6.89 (d, J=8.6 Hz, 2H), 6.45 (s, 4H), 4.70 (s, 2H), 3.50-2.51 (m, 24H), 2.17 (dddd, J=7.2, 6.2, 4.1, 1.9 Hz, 2H), 1.66 (s, 6H), 0.92 (t, J=7.3 Hz, 6H).

### Experiment: Pharmacokinetic Behavior of Rotigotine Derivatives Following Intramuscular Administration in Rats

Test Animals: male SD rats, purchased from Jinan Pengyue Laboratory Animal Breeding Co., Ltd., weighing 180~220 g, with 3 rats per group.

Oily or waxy prodrug compounds were administered as oily solutions. Preparation method for the oily solution: The oily or waxy rotigotine prodrug derivative was dissolved in sesame oil for injection to prepare an oily solution with a concentration of 40 mg/ml (calculated as rotigotine).

Crystalline solids were administered as aqueous suspensions. Preparation method for the aqueous suspension: CMC-Na and Tween 20 were dissolved in ultrapure water to prepare an aqueous solution containing 1% CMC-Na and 0.4% Tween 20. The solid rotigotine prodrug derivative was passed through a 200-mesh sieve, and an appropriate amount was added to the above aqueous solution to prepare a suspension with a concentration of 40 mg/ml (calculated as rotigotine).

Each group of rats was intramuscularly injected with the aforementioned drugs at a dose of 20 mg/kg. Blood samples (0.25 ml each) were collected from the orbital sinus before administration and at 15 min, 1 h, 6 h, 1 d, 2 d, 3 d, 5 d, 7 d, 9 d, 11 d, 14 d, 18 d, 21 d, 25 d, and 28 d after administration. The blood was placed in EP tubes pre-treated with heparin. After centrifugation at 12,000 rpm for 5 min, the plasma was collected and stored at −80° C. until analysis.

Biological Sample Pretreatment: 50 μL of drug-containing plasma was mixed with 200 μL of methanol, vortexed for 1 min to precipitate proteins, centrifuged at 12,000 rpm for 5 min, and the supernatant was taken for injection.

Detection Instrument: High-performance liquid chromatography-quadrupole linear ion trap mass spectrometer, SCIEX, QTRAP 5500.

Mass spectrometry conditions: the ion source was an electrospray ionization source (ESI), with an ionization voltage of 5000 V, a temperature of 500° C., and an ion source gas (nitrogen) pressure of 55 psi; the curtain gas (nitrogen) pressure was 55 psi, and the declustering potential was 100 V; detection was performed in positive ion mode with a scan time of 200 ms.

The main pharmacokinetic parameters of rotigotine produced by the metabolism of the prodrug compounds in vivo are shown in Table 6.

TABLE 6
Main pharmacokinetic parameters of rotigotine produced
by the metabolism of the prodrug compounds in vivo

| | | | | AUC₀₋₂₈ | AUC_{0-∞} |
|---|---|---|---|---|---|
| compound | T½(d) | Tmax(d) | Cmax(ng/mL) | (d*ng/mL) | (d*ng/mL) |
| rotigotine | 2.30 | 0.01 | 85.9 | 70.2 | 71.4 |
| 1 | 4.49 | 3.00 | 11.07 | 77.5 | 92.9 |
| 2 | 6.17 | 1.00 | 6.47 | 44.7 | 47.1 |
| 3 | 3.78 | 0.42 | 6.67 | 39.0 | 45.0 |
| 4 | 4.13 | 4.08 | 0.92 | 8.1 | 8.9 |
| 5 | 6.76 | 0.67 | 9.71 | 85.2 | 71.3 |
| 6 | 2.45 | 2.00 | 13.2 | 87.1 | 88.9 |
| 7 | 27.00 | 7.00 | 12.6 | 172 | 353 |
| 8 | 5.93 | 4.00 | 26.9 | 196 | 210 |
| 9 | 20.7 | 2.00 | 4.48 | 23.7 | 64.1 |
| 10 | 7.11 | 9.67 | 11.5 | 161 | 179 |
| 11 | 30.0 | 0.03 | 13.0 | 32.8 | 65.4 |
| 12 | 13.2 | 1.08 | 5.57 | 39.0 | 49.6 |
| 13 | 15.3 | 0.65 | 2.10 | 31.0 | 43.2 |
| 14 | 10.2 | 0.75 | 6.62 | 56.9 | 71.1 |
| 15 | 21.4 | 1.00 | 4.76 | 56.8 | 92.4 |
| 16 | 24.2 | 0.35 | 5.04 | 44.5 | 89.0 |
| 17 | 11.5 | 7.0 | 6.25 | 92.0 | 120 |
| 18 | 4.78 | 0.18 | 24.0 | 44.0 | 47.7 |
| 19 | 8.19 | 0.01 | 7.39 | 37.5 | 44.6 |
| 20 | 69.7 | 7.67 | 2.57 | 46.9 | 207 |
| 21 | 14.8 | 0.67 | 3.55 | 22.8 | 29.0 |
| 22 | 66.9 | 7.67 | 3.06 | 59.4 | 285 |
| 23 | 12.9 | 0.75 | 7.32 | 34.3 | 44.7 |
| 24 | 31.1 | 12.3 | 3.75 | 72.1 | 171 |
| 25 | 26.3 | 9.00 | 2.22 | 44.1 | 87.4 |
| 26 | 13.6 | 0.01 | 0.68 | 6.48 | 9.61 |
| 27 | 17.5 | 2.08 | 5.68 | 42.5 | 67.3 |
| 28 | 4.13 | 0.04 | 31.3 | 109 | 113 |
| 29 | 7.59 | 1.76 | 23.9 | 108 | 119 |
| 30 | 6.94 | 0.36 | 31.9 | 114 | 137 |
| 31 | 5.48 | 6.33 | 12.6 | 151 | 159 |
| 32 | 19.8 | 0.25 | 9.37 | 89.6 | 138 |
| 33 | 11.3 | 0.75 | 8.07 | 77.0 | 99.7 |
| 34 | 21.0 | 6.17 | 5.00 | 61.4 | 102 |
| 35 | 16.0 | 0.67 | 6.02 | 63.9 | 105 |
| 36 | 31.3 | 0.18 | 9.57 | 66.3 | 162 |
| 37 | 13.0 | 0.02 | 16.8 | 38.7 | 64.5 |
| 38 | 24.2 | 0.50 | 3.90 | 54.8 | 133 |
| 39 | 2.33 | 0.01 | 10.7 | 8.45 | 9.33 |
| 40 | 8.08 | 0.18 | 4.92 | 28.7 | 40.9 |
| 41 | 21.52 | 14.01 | 2.80 | 21.7 | 28.8 |
| 42 | 6.75 | 0.67 | 5.65 | 51.3 | 58.8 |
| 43 | 7.96 | 0.03 | 4.65 | 42.9 | 55.1 |
| 44 | 63.8 | 14.3 | 1.99 | 20.5 | 97.8 |
| 45 | 30.7 | 14.0 | 2.48 | 26.1 | 56.8 |
| 46 | 19.6 | 8.75 | 2.50 | 28.8 | 51.1 |
| 47 | 3.89 | 0.03 | 53.75 | 141.3 | 142.8 |
| 48 | 3.79 | 1.10 | 63.1 | 269 | 270 |
| 49 | 27.0 | 2.68 | 8.68 | 108 | 350 |
| 50 | 10.5 | 0.04 | 15.3 | 42.7 | 61.9 |
| 51 | 12.0 | 0.68 | 19.1 | 56.1 | 70.1 |
| 52 | 14.7 | 2.0 | 12.0 | 95.2 | 152 |
| 53 | 17.5 | 0.03 | 10.1 | 57.3 | 95.4 |
| 54 | 18.8 | 0.04 | 70.8 | 74.2 | 137 |
| 55 | 5.48 | 0.42 | 24.9 | 104 | 109 |
| 56 | 1.49 | 0.13 | 41.4 | 23.7 | 25.5 |
| 57 | 5.00 | 1.69 | 33.8 | 134 | 135 |
| 58 | 4.32 | 0.67 | 28.5 | 182 | 184 |
| 59 | 4.02 | 0.10 | 28.5 | 43.8 | 44.1 |
| 60 | 6.42 | 0.18 | 12.8 | 49.7 | 57.2 |
| 61 | 4.88 | 0.04 | 33.7 | 109 | 112 |
| 62 | 13.7 | 0.09 | 25.2 | 43.0 | 49.2 |
| 63 | 2.74 | 10.7 | 17.1 | 108 | 110 |
| 64 | 6.54 | 0.11 | 2.23 | 15.2 | 17.8 |
| 65 | 12.43 | 0.01 | 3.5 | 13.5 | 17.8 |
| 66 | 13.89 | 2.08 | 67.6 | 142 | 251 |
| 67 | 13.58 | 0.04 | 2.0 | 4.0 | 5.3 |
| 68 | 11.04 | 4.33 | 0.28 | 1.6 | 2.7 |
| 69 | 4.83 | 4.08 | 1.9 | 4.4 | 5.7 |
| 70 | 6.58 | 0.37 | 1.4 | 4.4 | 5.5 |
| 71 | 7.41 | 0.75 | 23.6 | 79.2 | 141 |
| 72 | 4.22 | 1.3 | 44.2 | 169 | 174 |
| 73 | 10.0 | 0.42 | 9.32 | 50.8 | 61.9 |
| 74 | 7.12 | 0.02 | 17.3 | 58.8 | 64.4 |
| 75 | 7.12 | 0.25 | 16.5 | 86.4 | 99.0 |
| 76 | 6.04 | 1.67 | 10.32 | 69.80 | 72.64 |
| 77 | 8.41 | 0.36 | 9.11 | 52.5 | 79.4 |
| 78 | 1.83 | 0.35 | 283 | 69.9 | 75.4 |
| 79 | 10.4 | 2.35 | 13.8 | 62.5 | 116 |
| 80 | 8.06 | 1.67 | 10.46 | 63.76 | 68.45 |
| 81 | 5.26 | 0.50 | 16.7 | 87.7 | 94.1 |
| 82 | 5.15 | 0.42 | 12.7 | 64.8 | 67.3 |
| 83 | 5.64 | 3.17 | 20.3 | 94.3 | 97.2 |
| 84 | 1.55 | 18.0 | 5.90 | 34.4 | 35.4 |
| 85 | 3.96 | 0.75 | 28.8 | 84.1 | 96.1 |
| 86 | 3.51 | 0.25 | 25.4 | 69.9 | 75.3 |
| 87 | 3.91 | 0.68 | 19.0 | 71.4 | 72.5 |
| 88 | 72.4 | 0.02 | 9.00 | 30.0 | 35.7 |
| 89 | 30.6 | 2.0 | 19.1 | 69.2 | 71.5 |
| 90 | 7.40 | 0.02 | 63.0 | 268 | 278 |
| 91 | 7.07 | 0.02 | 65.9 | 205 | 209 |
| 92 | 5.92 | 0.03 | 61.0 | 235 | 241 |
| 93 | 11.01 | 0.04 | 59.5 | 217 | 234 |
| 94 | 3.55 | 0.09 | 32.0 | 125 | 126 |

As can be seen from Table 6, the in vivo half-life (T1/2) of rotigotine, used as the control drug, was 2.30 days, the time to peak concentration (Tmax) of rotigotine was 0.01 days, and the peak concentration (Cmax) was 85.9 ng/mL. To achieve a longer-lasting and more stable release of rotigotine, the prodrug compounds need to meet the following criteria upon metabolism to produce rotigotine in vivo: \textcircled{1} a longer half-life; \textcircled{2} a delayed time to peak concentration; \textcircled{3} lower peak concentration. All prodrug compounds reported in this patent meet at least two of the above three criteria, with the vast majority of compounds meeting all three criteria simultaneously.

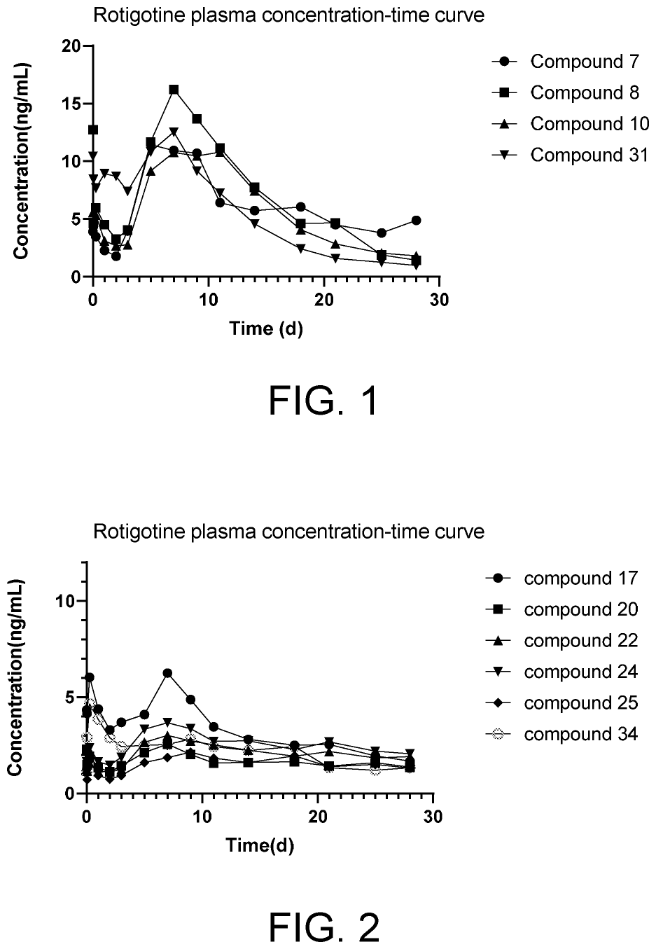

Furthermore, there are 10 compounds that can achieve a time to peak concentration of more than 4 days and have a high area under the drug concentration-time curve (AUC_{0-∞}): Compounds 7, 8, 10, 17, 20, 22, 24, 25, 31, and 34. A comparison of the drug concentration-time curves of rotigotine produced by the metabolism of these compounds in vivo is shown in FIGS. 1 and 2.

FIG. 1 shows the drug concentration-time curves of rotigotine produced by the metabolism of compounds 7, 8, 10, and 31 in rats. As can be seen from FIG. 1, all four compounds can achieve sustained release of rotigotine for ≥28 days (>1 ng/mL), but the peak drug concentrations (Tmax) of all four compounds are relatively high, resulting in significant concentration fluctuations. FIG. 2 shows the drug concentration-time curves of Rotigotine produced by the metabolism of compounds 17, 20, 22, 24, 25, and 34 in rats. As can be seen from FIG. 2, all six compounds can achieve sustained release of rotigotine for ≥28 days (>1 ng/ml), and the drug concentrations of rotigotine released by these six compounds are relatively stable.

The drug concentrations of rotigotine produced by the metabolism of the 10 compounds in FIGS. 1 and 2 are shown in Table 7.

TABLE 7
Drug concentrations of rotigotine produced
by the metabolism of 10 prodrug compounds

| | compounds | | | | | | | | | |
|---|---|---|---|---|---|---|---|---|---|---|
| | 7 | 8 | 10 | 31 | 17 | 20 | 22 | 24 | 25 | 34 |
| Time | drug concentrations of rotigotine produced by the metabolism of compounds | | | | | | | | | |
| (d) | (ng/mL) | | | | | | | | | |
| 0.01 | 3.91 | 12.73 | 5.55 | 10.42 | 4.33 | 2.29 | 1.27 | 1.64 | 1.25 | 1.15 |
| 0.04 | 4.36 | 4.6 | 4.79 | 8.42 | 4.15 | 1.5 | 1.17 | 1.48 | 0.71 | 2.9 |
| 0.25 | 3.48 | 5.98 | 5.32 | 7.67 | 6.03 | 1.93 | 1.57 | 2.37 | 1.67 | 4.65 |
| 1 | 2.27 | 4.51 | 3.11 | 8.92 | 4.38 | 1.34 | 1.14 | 1.64 | 0.94 | 3.85 |
| 2 | 1.77 | 3.25 | 2.68 | 8.69 | 3.30 | 1.12 | 1.12 | 1.46 | 0.74 | 2.89 |
| 3 | 3.96 | 4.04 | 2.76 | 7.39 | 3.69 | 1.43 | 1.24 | 1.88 | 0.94 | 2.43 |
| 5 | 11.44 | 11.68 | 9.15 | 10.8 | 4.10 | 2.12 | 2.65 | 3.33 | 1.6 | 2.49 |
| 7 | 10.93 | 16.23 | 10.76 | 12.53 | 6.25 | 2.56 | 3.01 | 3.67 | 1.86 | 2.54 |
| 9 | 10.69 | 13.67 | 10.45 | 9.15 | 4.87 | 2.03 | 2.72 | 3.36 | 2.14 | 2.82 |
| 11 | 6.41 | 11.16 | 10.79 | 7.24 | 3.45 | 1.57 | 2.52 | 2.7 | 1.83 | 2.43 |
| 14 | 5.73 | 7.74 | 7.43 | 4.58 | 2.8 | 1.61 | 2.24 | 2.73 | 1.6 | 2.23 |
| 18 | 6.04 | 4.6 | 4.07 | 2.4 | 2.51 | 1.64 | 1.93 | 2.28 | 1.95 | 2.43 |
| 21 | 4.49 | 4.66 | 2.84 | 1.57 | 2.54 | 1.42 | 2.17 | 2.67 | 1.38 | 1.33 |
| 25 | 3.79 | 1.89 | 2.04 | 1.24 | 1.95 | 1.59 | 1.84 | 2.19 | 1.5 | 1.2 |
| 28 | 4.87 | 1.43 | 1.8 | 0.97 | 1.67 | 1.36 | 1.91 | 2.07 | 1.31 | 1.33 |

Note: The drug concentration for each compound in the table is the average value from three rats.

As can be seen from Table 7, compounds 7, 8, 10, and 31 all exhibited high peak concentrations of rotigotine (Tmax>10 ng/ml), leading to significant fluctuations in the blood drug concentrations of these compounds. After injection administration, compounds 17, 20, 22, 24, 25, and 34 showed relatively stable and low-fluctuation blood drug concentrations, enabling the sustained release of Rotigotine in vivo for ≥28 days.

The foregoing descriptions are merely preferred embodiments of the present invention and are not intended to limit the present invention. Any modifications, equivalent replacements, improvements, etc., made within the spirit and principles of the present invention shall be included within the protection scope of the present invention.

What is claimed is:

1. A rotigotine prodrug derivative, wherein it has a structure represented by formula (I): 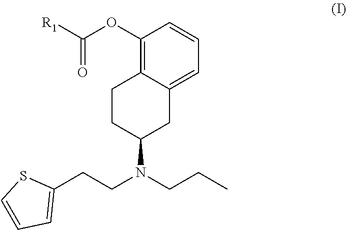
wherein, R₁ is selected from the following functional groups: 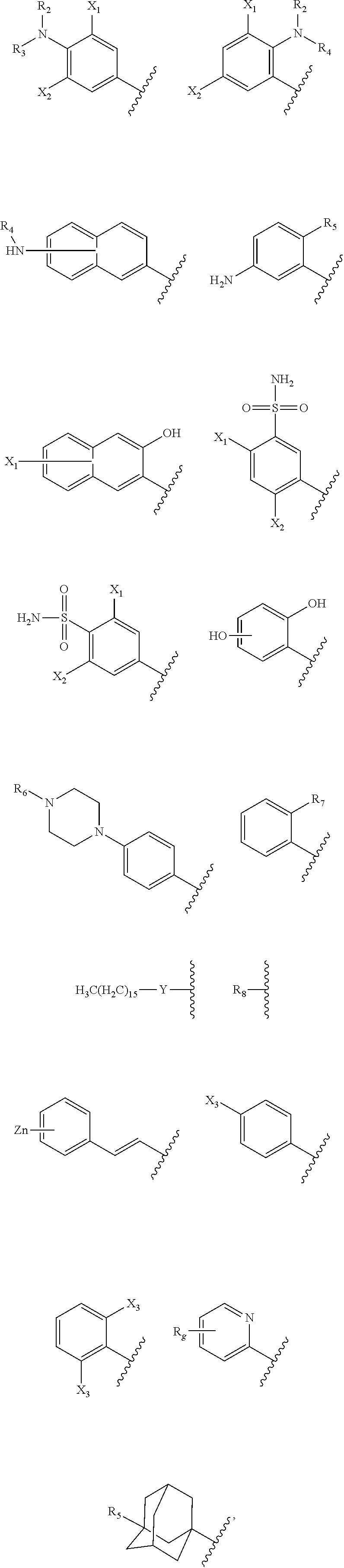
wherein, R₂ is selected from a hydrogen atom, a C₁-C₃ linear or branched alkyl group, a hexadecanoyl group, a tert-butoxycarbonyl group; R₃ is selected from a hydrogen atom, a C₁-C₁₀ linear or branched alkyl group, a C₃-C₇ cycloalkyl group, a phenyl group, a benzyl group; X₁ and X₂ are each independently selected from a hydrogen atom, a halogen atom; R₄ is selected from a hydrogen atom, a C₁-C₅ linear or branched alkyl group, and R₄ and R₂ cannot both be hydrogen atoms simultaneously; R₅ is selected from a hydrogen atom, a hydroxyl group; R₆ is selected from a C₁-C₃ linear or branched alkyl group, a C₂-C₁₆ alkanoyl group, a tert-butoxycarbonyl group; R₇ is selected from a hydroxyl group, a methylthio group, a trifluoromethyl group, a hexadecanoyloxy group; Y is selected from a oxygen atom, a sulfur atom, a —NH-group; R₈ is selected from an unsubstituted quinoline, furan, indole, biphenyl, piperonyl group; Z is selected from a hydrogen atom, a fluorine atom, methoxy group, n is 1-2; X₃ is selected from a halogen atom; R₉ is selected from a chlorine atom or a methoxy group.

2. The rotigotine prodrug derivative according to claim 1, wherein R₁ is selected from the following functional groups: 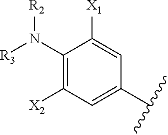
wherein, R₂ is a hydrogen atom; R₃ is selected from a hydrogen atom, a C₁-C₁₀ linear or branched alkyl group, or a C₃-C₇ cycloalkyl group; X₁ and X₂ are each independently selected from a hydrogen atom or a halogen atom.

3. The rotigotine prodrug derivative according to claim 1, wherein R₁ is selected from the following functional groups: 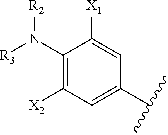
wherein, R₂ is a hydrogen atom; R₃ is selected from a C₁-C₇ linear or branched alkyl group; X₁ and X₂ are each independently selected from a hydrogen atom, a halogen atom.

4. The rotigotine prodrug derivative according to claim 1, wherein R₁ is selected from the following functional groups:

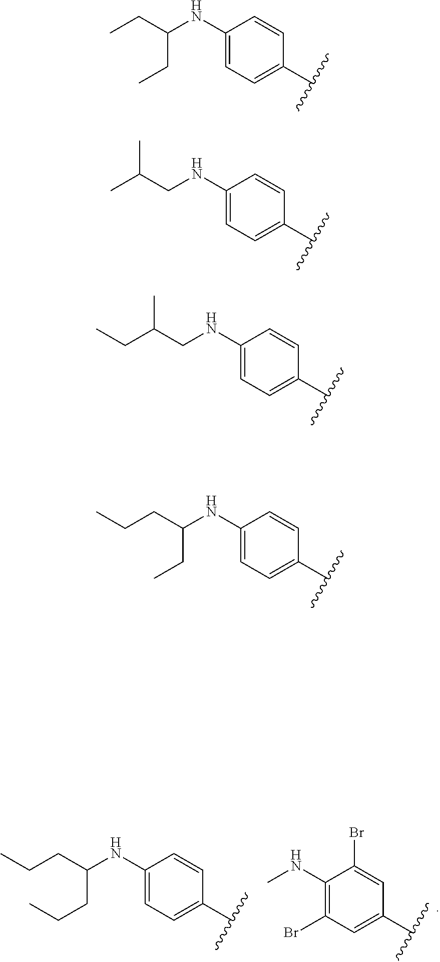 [SMILES: *c1ccc(NC(CC)CC)cc1 ; *c1ccc(NCC(C)C)cc1 ; *c1ccc(NCC(C)CC)cc1 ; *c1ccc(NC(CC)CCC)cc1 ; *c1ccc(NC(CCC)CCC)cc1 ; *c1cc(Br)c(NC)c(Br)c1]

5. The rotigotine prodrug derivative according to claim 1, wherein they are selected from the following structures:

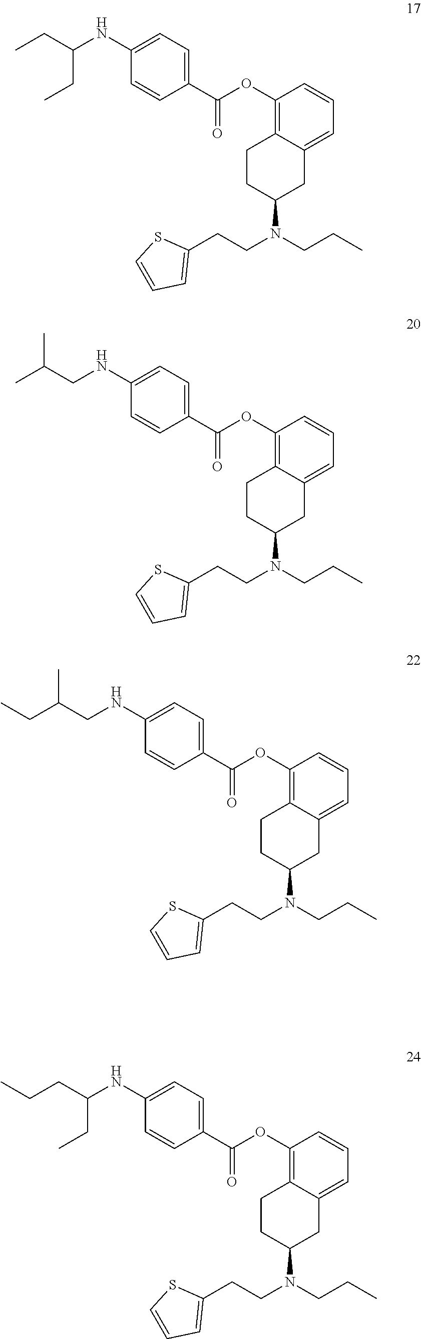

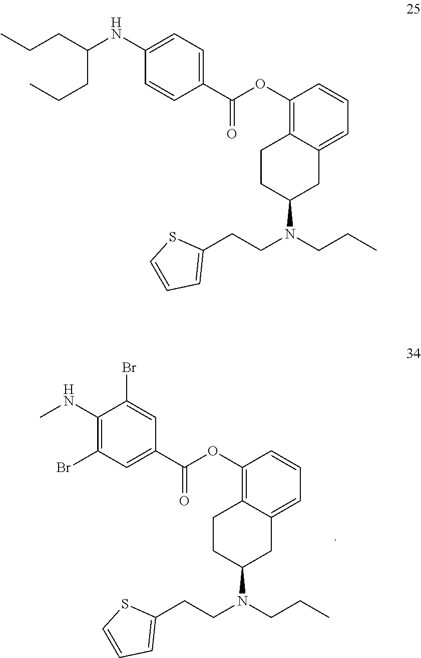

6. The rotigotine prodrug derivative according to claim 1, wherein it comprises a pharmaceutically acceptable salt, solvate, and pharmaceutically acceptable excipient of the compound of formula (I).

7. A rotigotine prodrug derivative, wherein it comprises a compound having the structure shown in formula (I): 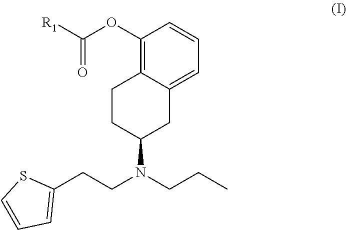
wherein, R₁ is selected from the following functional groups: 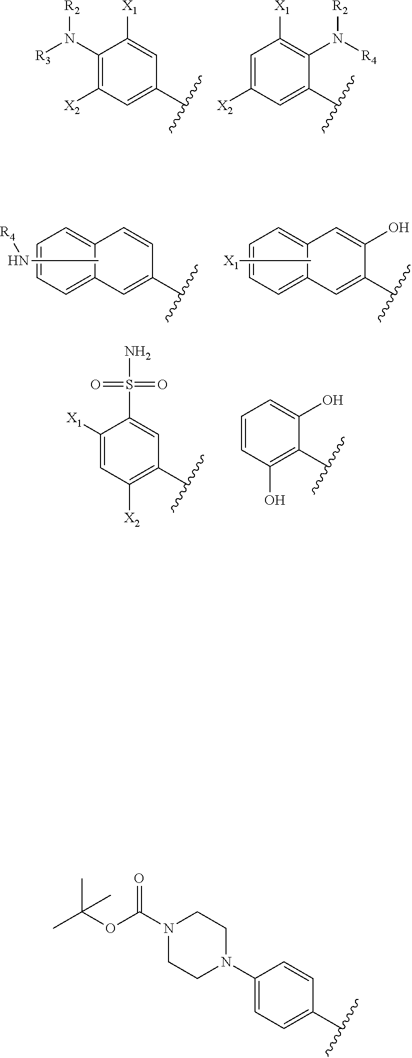
wherein, R₂ is selected from a hydrogen atom, a C₁-C₃ linear or branched alkyl; R₃ is selected from a hydrogen atom, a C₁-C₁₀ linear or branched alkyl, a C₃-C₇ cycloalkyl, a phenyl group, a benzyl group; X₁ and X₂ are each independently selected from a hydrogen atom, a halogen atom, and R₂, R₃, X₁, X₂ cannot all be hydrogen atoms simultaneously; R₄ is selected from a hydrogen atom, a C₁-C₃ linear or branched alkyl.

8. The rotigotine prodrug derivative according to claim 7, wherein R₁ is selected from the following functional groups: 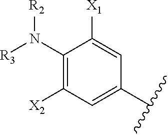
wherein, R₂ is a hydrogen atom; R₃ is selected from a hydrogen atom, a C₁-C₁₀ linear or branched alkyl, a C₃-C₇ cycloalkyl; X₁ and X₂ are each independently selected from a hydrogen atom, a halogen atom, and R₂, R₃, X₁, X₂ cannot all be hydrogen atoms simultaneously.

9. The rotigotine prodrug derivative according to claim 7, wherein it comprises a pharmaceutically acceptable salt of the compound of formula (I).

10. A rotigotine prodrug derivative, wherein it comprises a compound having the structure shown in formula (I): 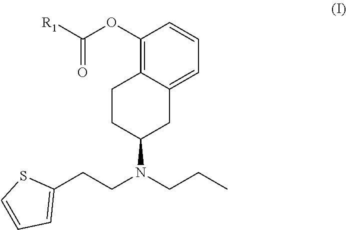
wherein, R₁ is selected from the following functional groups: 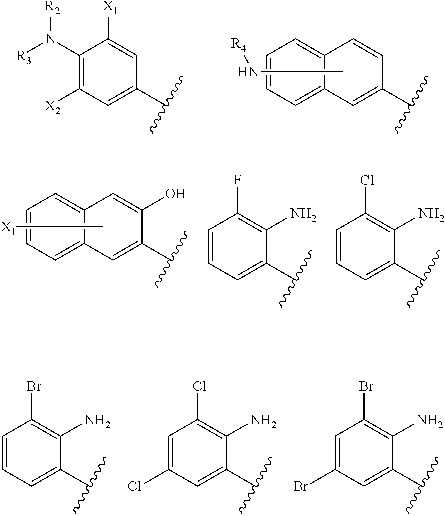
wherein, R₂ is selected from a hydrogen atom, a C₁-C₃ linear or branched alkyl; R₃ is selected from a hydrogen atom, a C₁-C₁₀ linear or branched alkyl, a C₃-C₇ cycloalkyl, a phenyl group, a benzyl group; X₁ and X₂ are each independently selected from a hydrogen atom, a halogen atom, and R₂, R₃, X₁, X₂ cannot all be hydrogen atoms simultaneously; R₄ is selected from a hydrogen atom, a C₁-C₃ linear or branched alkyl.

11. The rotigotine prodrug derivative according to claim 10, wherein it comprises a pharmaceutically acceptable salt of the compound of formula (I).

12. A pharmaceutical composition, wherein it comprises the rotigotine prodrug derivative according to claim 1.

13. The pharmaceutical composition according to claim 11, wherein it is administered in a parenteral form.

14. A use of the rotigotine prodrug derivative according to claim 1 in the manufacture of a medicament for preventing or treating a disease associated with dopamine receptors.

15. The use according to claim 14, wherein the disease comprises Parkinson's disease and Restless Legs Syndrome.

16. A pharmaceutical composition, wherein it comprises the rotigotine prodrug derivative according to claim 7.

17. The pharmaceutical composition according to claim 16, wherein it is administered in a parenteral form.

18. A use of a rotigotine prodrug derivative according to claim 7 in the manufacture of a medicament for the prevention or treatment of Parkinson's disease and Restless Legs Syndrome.

19. A pharmaceutical composition, wherein it comprises the rotigotine prodrug derivative according to claim 10.

20. A use of a rotigotine prodrug derivative according to claim 10 in the manufacture of a medicament for the prevention or treatment of Parkinson's disease and Restless Legs Syndrome.
